(19) United States
(12) Patent Application Publication
(10) Pub. No.: US 2019/0062277 A1
(43) Pub. Date: Feb. 28, 2019

(54) FARNESOID X RECEPTOR AGONISTS AND USES THEREOF

(71) Applicant: Metacrine, Inc., San Diego, CA (US)

(72) Inventors: Nicholas D. SMITH, San Diego, CA (US); Steven P. GOVEK, San Diego, CA (US); Karensa L. Douglas, San Diego, CA (US)

(21) Appl. No.: 15/758,710

(22) PCT Filed: Sep. 16, 2016
(86) PCT No.: PCT/US2016/052274

§ 371 Date: Mar. 8, 2018

Related U.S. Application Data
(60) Provisional application No. 62/219,427, filed on Sep. 16, 2015.
(60) Provisional application No. 62/333,560, filed on May 9, 2016.

(51) Int. Cl. C07D 213/40; C07D 207/09; C07D 205/04; A61K 31/4418; A61K 31/451; A61K 31/402; A61K 31/397; C07D 211/26; C07C 233/63; A61K 31/196; A61K 31/216; C07C 233/54; A61P 1/16; A61K 45/06

(52) U.S. Cl. CPC C07D 213/40; C07D 207/09; C07D 205/04; A61K 31/4418; A61K 31/451; A61K 31/402; A61K 45/06; C07D 211/26; C07C 233/63; A61K 31/196; A61K 31/216; C07C 233/54; A61P 1/16; A61K 31/397

(57) ABSTRACT

Described herein are compounds that are famesoid X receptor agonists, methods of making such compounds, pharmaceutical compositions and medicaments comprising such compounds, and methods of using such compounds in the treatment of conditions, diseases, or disorders associated with famesoid X receptor activity.

### CROSS-REFERENCE

This application claims benefit of U.S. Provisional Patent Application No. 62/219,427 filed on Sep. 16, 2015, and U.S. Provisional Patent Application No. 62/333,560 filed on May 9, 2016, each incorporated herein by reference in its entirety.

### FIELD OF THE INVENTION

Described herein are compounds that are famesoid X receptor agonists, methods of making such compounds, pharmaceutical compositions and medicaments comprising such compounds, and methods of using such compounds in the treatment of conditions, diseases, or disorders associated with famesoid X receptor activity.

### BACKGROUND OF THE INVENTION

Famesoid X receptor (FXR) is a nuclear receptor highly expressed in the liver, intestine, kidney, adrenal glands, and adipose tissue. FXR regulates a wide variety of target genes involved in the control of bile acid synthesis and transport, lipid metabolism, and glucose homeostasis. FXR agonism is a treatment modality for many metabolic and liver conditions.

### SUMMARY OF THE INVENTION

In one aspect, described herein are famesoid X receptor agonists and uses thereof.

In some embodiments, the famesoid X receptor agonists described herein have the structure of Formula (I), or a pharmaceutically acceptable salt thereof:

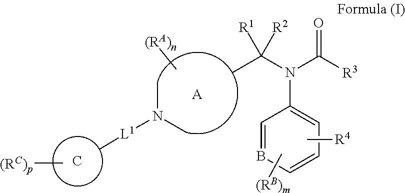

wherein,
R¹ and R² are each independently selected from H, D, F, C₁-C₄alkyl, or C₁-C₄fluoroalkyl;
or R¹ and R² are taken together with the carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NR¹⁰C(═O)R¹¹, —NR¹⁰C(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, —N(R¹⁰)₂, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
L¹ is —X¹-L²- or -L²-X¹—;
   X¹ is absent, —S(═O)—, —S(═O)₂—, —C(═O)—, —OC(═O)—, —NR¹⁰C(═O)—, or —NR¹⁰S(O)₂—,
   L² is absent or substituted or unsubstituted C₁-C₄alkylene;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R³)(R⁶)—O—;
      R⁵ and R⁷ are each independently selected from H, D, C₁-C₄alkyl and C₃-C₆cycloalkyl;
      or R³ and R⁷ are taken together with the intervening atoms to form a double bond;
      or R⁵ and R⁷ are taken together with the intervening atoms to form an epoxide or an substituted or unsubstituted C₃-C₆cycloalkyl;
      R⁶ and R⁸ are each independently selected from H, D, C₁-C₄alkyl or C₃-C₆cycloalkyl;
   Y is —CH₂OR⁹, —C(═O)OR⁹,

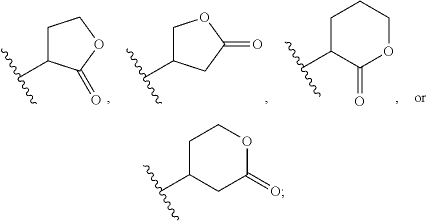 [SMILES: *C1CCOC1=O ; *C1COC(=O)C1 ; *C1CCCOC1=O ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
ring A is a monocyclic C₂-C₈heterocycloalkyl containing 1 N atom in the ring, or bicyclic C₅-C₈heterocycloalkyl;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)R¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

In some embodiments, the famesoid X receptor agonists described herein have the structure of Formula (II), or a pharmaceutically acceptable salt or solvate thereof:

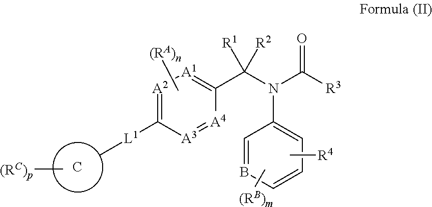

wherein,
A¹ is CR^{A};
or A¹ is N if at least one of A², A³, or A⁴ is N;
A² is CR^{A} or N;
A³ is CR^{A} or N;
A⁴ is CR^{A};
or A⁴ is N if at least one of A¹, A², or A³ is N
provided that at least one of A¹, A², A³, or A⁴ is N;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
R¹ and R² are each independently selected from H, D, F, C₁-C₄alkyl, or C₁-C₄fluoroalkyl;
or R¹ and R² are taken together with die carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹—C(═O)N(R¹⁰)₂, —OC(═O)N(R¹⁰)₂, —NR¹⁰C(═O)R¹¹, —NR¹⁰C(═O)R¹ unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂, —NR¹⁰—, —C(═O)—, —C(═O)NH—, —NHC(O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, —N(R¹⁰)₂, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
   R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
   R⁵ and R⁷ are each independently selected from H, D, C₁-C₄alkyl and C₃-C₆cycloalkyl;
   or R⁵ and R⁷ are taken together with the intervening atoms to form a double bond;
   or R⁵ and R⁷ are taken together with the intervening atoms to form an epoxide or an substituted or unsubstituted C₃-C₆cycloalkyl;
   R⁶ and R⁸ are each independently selected from H, D, C₁-C₄alkyl or C₃-C₆cycloalkyl;
   Y is-CH₂OR⁹, —C(═O)OR⁹,

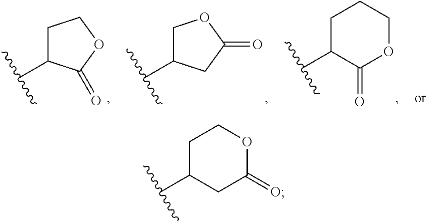 [SMILES: *C1CCOC1=O ; *C1COC(=O)C1 ; *C1CCCOC1=O ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
L¹ is —X¹-L²- or -L²-X¹—;
   X¹ is absent, —O—, —S—, —S(═O)₂—, —S(═O)₂—, —S(═O)₂NR¹⁰—, —CH₂—, —CH═CH—, —C≡C—, —C(═O)—, —C(═)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
   L² is absent or substituted or unsubstituted C₁-C₄alkylene;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic N-containing heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)R¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

In some embodiments, the famesoid X receptor agonists described herein have the structure of Formula (III), or a pharmaceutically acceptable salt or solvate thereof:

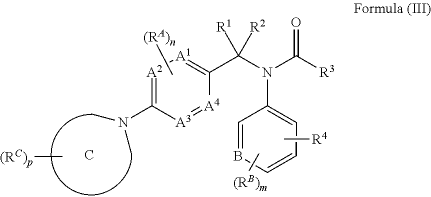

wherein,
ring C is a 5-membered N-containing heteroaryl, or a N-containing C₂-C₈heterocycloalkyl.

In some embodiments, the famesoid X receptor agonists described herein have the structure of Formula (IV), or a pharmaceutically acceptable salt or solvate thereof:

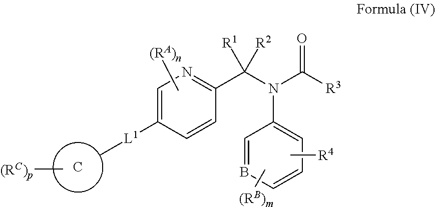

wherein,
R¹ and R² are each independently selected from H, D, F, C₁-C₄alkyl, or C₁-C₄fluoroalkyl;
or R¹ and R² are taken together with die carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NR¹⁰C(═O)R¹¹, —NR¹⁰C(═O)R¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═)₂—, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, —N(R¹⁰), unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
   R⁵ and R⁷ are each independently selected from H, D, C₁-C₄alkyl and C₃-C₆cycloalkyl;
   or R⁵ and R⁷ are taken together with the intervening atoms to form a double bond;
   or R⁵ and R⁷ are taken together with the intervening atoms to form an epoxide or an substituted or unsubstituted C₃-C₆cycloalkyl;
   R⁶ and R⁸ are each independently selected from H, D, C₁-C₄alkyl or C₃-C₆cycloalkyl;
   Y is-CH₂OR⁹, —C(═O)OR⁹,

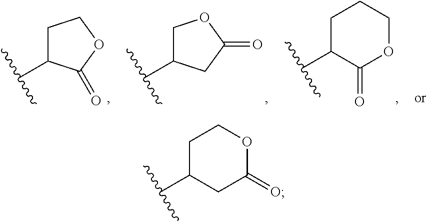 [SMILES: *C1CCOC1=O ; *C1COC(=O)C1 ; *C1CCCOC1=O ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
L¹ is —X¹-L²- or -L²-X¹—;
   X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
   L² is absent or substituted or unsubstituted C₁-C₄alkylene;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
ring C is monocyclic carbocycle, bicyclic carbocycle, monocyclic N-containing heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl, and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4,
provided that the compound is not methyl (E)-3-(3-(N-((5-(4-(dimethylamino)phenyl)-pyridin-2-yl)methyl)cyclohexanecarboxamido)phenyl)acrylate.

In some embodiments, the famesoid X receptor agonists described herein have the structure of Formula (V), or a pharmaceutically acceptable salt or solvate thereof:

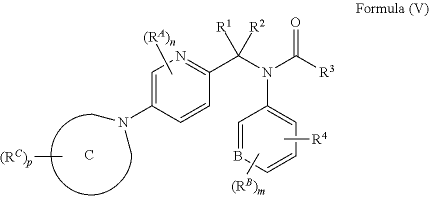

wherein,
ring C is a 5-membered N-containing heteroaryl, or a N-containing C₂-C₈heterocycloalkyl.

In some embodiments, the famesoid X receptor agonists described herein have the structure of Formula (VI), or a pharmaceutically acceptable salt or solvate thereof:

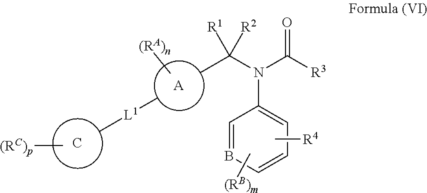

wherein,
R¹ and R² are each independently selected from H, D, F, C₁-C₄alkyl, or C₁-C₄fluoroalkyl;
or R¹ and R² are taken together with die carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NR¹⁰C(═O)R¹¹, —NR¹⁰C(═O)OR¹ unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₅-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═)₂—, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, —N(R¹⁰)₂, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
   R⁵ and R⁷ are each independently selected from H, D, C₁-C₄alkyl and C₃-C₆cycloalkyl;
   or R⁵ and R⁷ are taken together with the intervening atoms to form a double bond;
   or R⁵ and R⁷ are taken together with the intervening atoms to form an epoxide or an substituted or unsubstituted C₃-C₆cycloalkyl;
   R⁶ and R⁸ are each independently selected from H, D, C₁-C₄alkyl or C₃-C₆cycloalkyl;
   Y is —CH₂OR⁹, —C(═O)OR⁹,

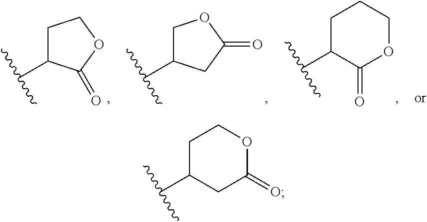 [SMILES: *C1CCOC1=O ; *C1COC(=O)C1 ; *C1CCCOC1=O ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
L¹ is —X¹-L²- or -L²-X¹—;
   X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
   L² is absent or substituted or unsubstituted C₁-C₄alkylene;
ring A is a monocyclic 5-membered heteroarylene;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰), —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

In some embodiments, the famesoid X receptor agonists described herein have the structure of Formula (VII), or a pharmaceutically acceptable salt or solvate thereof:

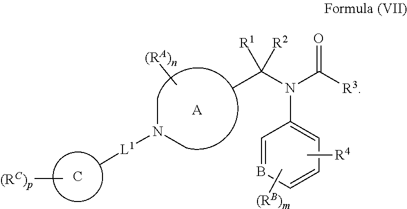

In some embodiments, the famesoid X receptor agonists described herein have the structure of Formula (VIII), or a pharmaceutically acceptable salt or solvate thereof:

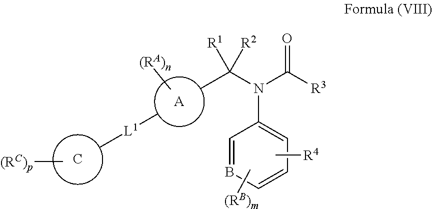

wherein,
R¹ and R² are each independently selected from H, D, F, C₁-C₄alkyl, or C₁-C₄fluoroalkyl;
or R¹ and R² are taken together with die carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NR¹⁰C(═O)R¹¹, —NR¹⁰C(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂—, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, —N(R¹⁰)₂, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
   R⁵ and R⁷ are each independently selected from H, D, C₁-C₄alkyl and C₃-C₆cycloalkyl;
   or R⁵ and R⁷ are taken together with the intervening atoms to form a double bond;
   or R⁵ and R⁷ are taken together with the intervening atoms to form an epoxide or an substituted or unsubstituted C₃-C₆cycloalkyl;
   R⁶ and R⁸ are each independently selected from H, D, C₁-C₄alkyl or C₃-C₆cycloalkyl;
   Y is-CH₂OR⁹, —C(═O)OR⁹,

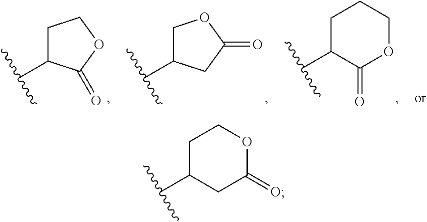 [SMILES: *C1CCOC1=O ; *C1COC(=O)C1 ; *C1CCCOC1=O ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
L¹ is —X¹-L²- or -L²-X¹—;
   X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
   L² is absent or substituted or unsubstituted C₁-C₄alkylene;
ring A is a C₃-C₁₀cycloalkyl;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰), —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

Any combination of the groups described above for the various variables is contemplated herein. Throughout the specification, groups and substituents thereof are chosen by one skilled in the field to provide stable moieties and compounds.

In one aspect, described herein is a pharmaceutical composition comprising a compound described herein, or a pharmaceutically acceptable salt, or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In another aspect, described herein is a method of treating a disease or condition in a mammal that would benefit from FXR agonism comprising administering a compound as described herein, or pharmaceutically acceptable salt, or solvate thereof, to the mammal in need thereof. In some embodiments, the disease or condition is a metabolic condition. In some embodiments, the disease or condition is a liver condition.

In some embodiments, the compound is administered to the mammal by intravenous administration, subcutaneous administration, oral administration, inhalation, nasal administration, dermal administration, or ophthalmic administration.

In one aspect, described herein is a method of treating or preventing any one of the diseases or conditions described herein comprising administering a therapeutically effective amount of a compound described herein, or a pharmaceutically acceptable salt, or solvate thereof, to a mammal in need thereof.

In one aspect, described herein is a method for the treatment or prevention of a metabolic or liver condition in a mammal comprising administering a therapeutically effective amount of a compound described herein, or a pharmaceutically acceptable salt, or solvate thereof, to the mammal in need thereof. In other embodiments, the metabolic or liver condition is amenable to treatment with a FXR agonist. In some embodiments, the method further comprises administering a second therapeutic agent to the mammal in addition to the compound described herein, or a pharmaceutically acceptable salt, or solvate thereof.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by inhalation; and/or (e) t administered by nasal administration; or and/or (f) administered by injection to the mammal; and/or (g) administered topically to the mammal; and/or (h) administered by ophthalmic administration; and/or (i) administered rectally to the mammal; and/or (j) adminstered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the aforementioned aspects involving the treatment of a disease or condition are further embodiments comprising administering at least one additional agent in addition to the administration of a compound described herein, or a pharmaceutically acceptable salt thereof. In various embodiments, each agent is administered in any order, including simultaneously.

In any of the embodiments disclosed herein, the mammal or subject is a human.

In some embodiments, compounds provided herein are administered to a human.

In some embodiments, compounds provided herein are orally administered.

In some embodiments, described herein is method of treating or preventing a metabolic disorder in a subject, comprising: administering to a gastrointestinal tract of the subject a therapeutically effective amount of one or more of the compounds described herein, or a pharmaceutically acceptable salt or solvate thereof, thereby activating famesoid X receptors (FXR) in the intestines, and treating or preventing a metabolic disorder in the subject. In some embodiments, the compound's absorption is preferentially restricted to within the intestines. In some embodiments, the method substantially enhances FXR target gene expression in the intestines while not substantially enhancing FXR target gene expression in the liver or kidney. In some embodiments, the method substantially enhances FXR target gene expression in the intestines while minimizing systemic plasma levels of the delivered compound. In some embodiments, the method substantially enhances FXR target gene expression in the intestines and the liver while minimizing systemic plasma levels of the delivered compound. In some embodiments, the method substantially enhances FXR target gene expression in the intestines while not substantially enhancing FXR target gene expression in the liver or kidney, and while minimizing systemic plasma levels. In some embodiments, the method substantially enhances FXR target gene expression in the intestines and the liver and provides sustained systemic plasma levels of the delivered compound. In some embodiments, the method reduces or prevents diet-induced weight gain. In some embodiments, the method increases a metabolic rate in the subject. In some embodiments, the increasing the metabolic rate comprises enhancing oxidative phosphorylation in the subject. In some embodiments, the method further comprises improving glucose and/or lipid homeostasis in the subject. In some embodiments, the method results in no substantial change in food intake and/or fat consumption in the subject. In some embodiments, the method results in no substantial change in appetite in the subject. In some embodiments, the metabolic disorder is selected from obesity, diabetes, insulin resistance, dyslipidemia, or any combination thereof. In some embodiments, the metabolic disorder is non-insulin dependent diabetes mellitus. In some embodiments, the method protects against diet-induced weight gain, reduces inflammation, enhances thermogenesis, enhances insulin sensitivity in the liver, reduces hepatic steatosis, promotes activation of BAT, decreases blood glucose, increases weight loss, or any combination thereof. In some embodiments, the method enhances insulin sensitivity in the liver and promotes brown adipose tissue (BAT) activation. In some embodiments, the method further comprises administering to the subject an insulin sensitizing drug, an insulin secretagogue, an alpha-glucosidase inhibitor, a glucagon-like peptide (GLP) agonist, a dipeptidyl peptidase-4 (DPP-4) inhibitor, nicotinamide ribonucleoside, an analog of nicotinamide ribonucleoside, or combinations thereof.

In some embodiments, described herein is a method of treating or preventing inflammation in an intestinal region of a subject, comprising: administering to a gastrointestinal tract of the subject a therapeutically effective amount of one or more of the compounds described herein, or a pharmaceutically acceptable salt or solvate thereof, thereby activating FXR receptors in the intestines, and thereby treating or preventing inflammation in the intestinal region of the subject. In some embodiments, the compound's absorption is preferrentially restricted to within the intestines. In some embodiments, the method substantially enhances FXR target gene expression in the intestines while not substantially enhancing FXR target gene expression in the liver or kidney. In some embodiments, the inflammation is associated with a clinical condition selected from necrotizing enterocolitis, gastritis, ulcerative colitis, Crohn's disease, inflammatory bowel disease, irritable bowel syndrome, gastroenteritis, radiation induced enteritis, pseudomembranous colitis, chemotherapy induced enteritis, gastroesophageal reflux disease (GERD), peptic ulcer, non-ulcer dyspepsia (NUD), celiac disease, intestinal celiac disease, post-surgical inflammation, gastric carcinogenesis, or any combination thereof. In some embodiments, the method further comprises administering a therapeutically effective amount of an antibiotic therapy to the subject, wherein the method treats or prevents inflammation associated with pseudomembranous colitis in the subject. In some embodiments, the method further comprises administering to the subject a therapeutically effective amount of an oral corticosteroid, other anti-inflammatory or immunomodulatory therapy, nicotinamide ribonucleoside, an analog of nicotinamide ribonucleoside, or combinations thereof. In some embodiments, the method increases HSL phosphorylation and β3-adrenergic receptor expression. In some embodiments, a serum concentration of the compound in the subject remains below its EC₅₀ following administration of the compound.

In some embodiments, described herein is a method of treating or preventing a cell proliferation disease in a subject, comprising administering to a gastrointestinal tract of the subject a therapeutically effective amount of one or more of the compounds described herein or a pharmaceutically acceptable salt or solvate thereof. In some embodiments, the cell proliferation disease is an adenocarcinoma. In some embodiments, the adenocarcinoma is a colon cancer. In some embodiments, the treating the adenocarcinoma reduces the size of the adenocarcinoma, the volume of the adenocarcinoma, the number of adenocarcinomas, cachexia due to the adenocarcinoma, delays progression of the adenocarcinoma, increases survival of the subject, or combinations thereof. In some embodiments, the method further comprises administering to the subject an additional therapeutic compound selected from the group consisting of a chemotherapeutic, a biologic, a radiotherapeutic, or combinations thereof.

In some embodiments, described herein is a method of treating or preventing a liver disease or condition in a subject, comprising administering to the subject a therapeutically effective amount of one or more of the compounds described herein, or a pharmaceutically acceptable salt or solvate thereof. In some embodiments, the liver disease or condition is an alcoholic or non-alcoholic liver disease. In some embodiments, the liver disease or condition is primary biliary cirrhosis, primary sclerosing cholangitis, cholestasis, nonalcoholic steatohepatitis (NASH), or nonalcoholic fatty liver disease (NAFLD). In some embodiments, the alcoholic liver disease or condition is fatty liver (steatosis), cirrhosis, or alcoholic hepatitis. In some embodiments, the non-alcoholic liver disease or condition is nonalcoholic steatohepatitis (NASH), or nonalcoholic fatty liver disease (NAFLD). In some embodiments, the non-alcoholic liver disease or condition is intrahepatic cholestasis or extrahepatic cholestasis.

Articles of manufacture, which include packaging material, a compound described herein, or a pharmaceutically acceptable salt thereof, within the packaging material, and a label that indicates that the compound or composition, or pharmaceutically acceptable salt, pharmaceutically active metabolite, pharmaceutically acceptable prodrug, or pharmaceutically acceptable solvate thereof, is used for the treatment, prevention or amelioration of one or more symptoms of a disease or condition that would benefit from FXR agonism, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

The nuclear hormone receptor famesoid X receptor (also known as FXR or nuclear receptor subfamily 1, group H, member 4 (NR1H4)) (OMIM: 603826) functions as a regulator for bile acid metabolism. FXR is a ligand-activated transcriptional receptor expressed in diverse tissues including the adrenal gland, kidney, stomach, duodenum, jejunum, ileum, colon, gall bladder, liver, macrophages, and white and brown adipose tissue. Bile acids function as endogenous ligands for FXR such that enteric and systemic release of bile acids induces FXR-directed changes in gene expression networks. Bile acids are the primary oxidation product of cholesterol, and in some cases, upon secretion into the intestines, are regulators of cholesterol absorption. The rate-limiting step for conversion of cholesterol into bile acids is catalyzed by cytochrome p450 enzyme cholesterol 7-α-hydroxylase (CYP7A1) and occurs in the liver. Activation of FXR represses the transcription of CYP7A1 by increasing the expression level of the hepatic small heterodimer partner (SHP) (also known as nuclear receptor subfamily 0, group B, member 2; or NR0B2) and intestinal expression of fibroblast growth factor 15 (FGF15) in mice and fibroblast growth factor 19 (FGF19) in human. SHP represses the liver receptor homolog (LRH-1), a nuclear receptor necessary for CYP7A1 gene expression, through its interaction with LRH-1 to form a non-functional heterodimer. In some cases, FGF15/19 released from the intestine then activates the fibroblast growth factor receptor 4 in the liver, leading to activation of the mitogen-activated protein kinase (MAPK) signaling pathway which suppress Cyp7A1.

In some embodiments, elevated levels of bile acids have been associated with insulin resistance. For example, insulin resistance sometimes leads to a decreased uptake of glucose from the blood, and increased de novo glucose production in the liver. In some instances, intestinal sequestration of bile acids has been shown to improve insulin resistance by promoting the secretion of glucagon-like peptide-1 (GLP1) from intestinal L-cells. GLP-1 is an incretin derived from the transcription product of the proglucagon gene. It is released in response to the intake of food and exerts control in appetite and gastrointestinal function, and promotes insulin secretion from the pancreas. The biologically active forms of GLP-1 include GLP-1-(7-37) and GLP-1-(7-36)NH₂, which result from selective cleavage of the proglucagon molecule. In such cases, activation of FXR leading to decreased production of bile acids correlates to a decrease in insulin resistance.

In some embodiments, the activation of FXR also correlates to the secretion of pancreatic polypeptide-fold such as peptide YY (PYY or PYY3-36). In some instances, peptide YY is a gut hormone peptide that modulates neuronal activity within the hypothalamic and brainstem, regions of the brain involved in reward processing. In some instances, reduced level of PYY correlates to increased appetite and weight gain.

In some instances, the activation of FXR indirectly leads to a reduction of plasma triglycerides. The clearance of triglycerides from the bloodstream is due to lipoprotein lipase (LPL). LPL activity is enhanced by the induction of its activator apolipoprotein CII, and the repression of its inhibitor apolipoprotein CHI in the liver occurs upon FXR activation.

In some cases, the activation of FXR further modulates energy expenditure such as adipocyte differentiation and function. Adipose tissue comprises adipocytes or fat cells. In some instances, adipocytes are further differentiated into brown adipose tissue (BAT) or white adipose tissue (WAT). The function of BAT is to generate body heat, while WAT functions as fat storing tissues.

In some instances, FXR is widely expressed in the intestine. In some cases, the activation of FXR has been shown to induce the expression and secretion of FGF19 (or FGF15 in mouse) in the intestine. FGF19 is a hormone that regulates bile acid synthesis as well as exerts an effect on glucose metabolism, lipid metabolism, and on energy expenditure. In some instances, FGF19 has also been observed to modulate adipocyte function and differentiation. Indeed, a study has shown that the administration of FGF19 to high-fat diet-fed mice increased energy expenditure, modulated adipocytes differentiation and function, reversed weight gain, and improved insulin resistance (see, Fu et al., “Fibroblast growth factor 19 increases metabolic rate and reverses dietary and leptin-deficient diabetes.” Endocrinology 145:2594-2603 (2004)).

In some cases, intestinal FXR activity has also been shown to be involved in reducing overgrowth of the microbiome, such as during feeding (Li et al., Nat Commun 4:2384, 2013). For example, a study had shown that activation of FXR correlated with increased expression of several genes in the ileum such as Ang2, iNos, and 1118, which have established antimicrobial actions (Inagaki et al., Proc Natl Acad Sci USA 103:3920-3925, 2006).

G protein-coupled bile acid receptor 1 (also known as GPBAR2, GPCR19, membrane-type receptor for bile acids or M-BAR, or TGR5) is a cell surface receptor for bile acids. Upon activation with bile acid, TGR5 induces the production of intracellular cAMP, which then triggers an increase in triiodothyronine due to the activation of deiodinase (DIO2) in BAT, resulting in increased energy expenditure.

Hence in some embodiments, regulation of metabolic processes such as bile acid synthesis, bile-acid circulation, glucose metabolism, lipid metabolism, or insulin sensitivity is modulated by the activation of FXR. Furthermore, in some embodiments, dis-regulation of metabolic processes such as bile acid synthesis, bile-acid circulation, glucose metabolism, lipid metabolism, or insulin sensitivity results in metabolic diseases such as diabetes or diabetes-related conditions or disorders, alcoholic or non-alcoholic liver disease or condition, intestinal inflammation, or cell proliferative disorders.

Disclosed herein, in certain embodiments, are compounds that have activity as FXR agonists. In some embodiments, the FXR agonists described herein are structurally distinct from bile acids, other synthetic FXR ligands, and other natural FXR ligands.

In some embodiments, also disclosed herein are methods of treating or preventing a metabolic disorder, such as diabetes, obesity, impaired glucose tolerance, dyslipidemia, or insulin resistance by administering a therapeutically effective amount of an FXR agonist. In some instances, the compounds are administered to the GI tract of a subject.

In additional embodiments, disclosed herein are methods for treating or preventing alcoholic or non-alcoholic liver disease or conditions (e.g., cholestasis, primary biliary cirrhosis, steatosis, cirrhosis, alcoholic hepatitis, non-alcoholic steatohepatitis (NASH), non-alcoholic fatty liver disease (NAFLD), primary sclerosing cholangitis (PSC) or elevated liver enzymes) by administering a therapeutically effective amount of an FXR agonist to a subject in need thereof (e.g., via the GI tract). In additional embodiments, disclosed herein include methods for treating or preventing cholestasis, cirrhosis, primary biliary cirrhosis, non-alcoholic steatohepatitis (NASH), non-alcoholic fatty liver disease (NAFLD), or primary sclerosing cholangitis (PSC) by administering a therapeutically effective amount of an FXR agonist to a subject in need thereof. In some embodiments, disclosed herein include methods for treating or preventing cholestasis by administering a therapeutically effective amount of an FXR agonist to a subject in need thereof. In some embodiments, disclosed herein include methods for treating or preventing primary biliary cirrhosis by administering a therapeutically effective amount of an FXR agonist to a subject in need thereof. In some embodiments, disclosed herein include methods for treating or preventing NASH by administering a therapeutically effective amount of an FXR agonist to a subject in need thereof. In some embodiments, disclosed herein include methods for treating or preventing NAFLD by administering a therapeutically effective amount of an FXR agonist to a subject in need thereof.

In further embodiments, disclosed herein include methods for treating or preventing inflammation in the intestines and/or a cell proliferative disorder, such as cancer, by administering a therapeutically effective amount of an FXR agonist to a subject in need thereof (e.g., via the GI tract).

In still further embodiments, disclosed herein include FXR agonists that modulate one or more of the proteins or genes associated with a metabolic process such as bile acid synthesis, glucose metabolism, lipid metabolism, or insulin sensitivity, such as for example, increase in the activity of FGF19 (FGF15 in mouse), increase in the secretion of GLP-1, or increase in the secretion of PYY.

Metabolic Disorders

Disclosed herein, in certain embodiments, are methods of treating a metabolic disorder in a subject in need thereof. Also described herein include methods of preventing a metabolic disorder in a subject in need thereof. In some instances, these methods include administering to the subject in need thereof a therapeutically effective amount of one or more of the compounds disclosed herein. In some instances, the one or more compounds disclosed herein are absorbed in the gastrointestinal (GI) tract. In additional instances, the one or more disclosed compounds absorbed in the GI tract activates FXR receptors thereby treating or preventing a metabolic disorder in the subject.

In some embodiments, the disclosed compounds demonstrate systemic exposure. In some instances, the disclosed compounds have local exposure in the intestines, but limited exposure in the liver or systemically. In some embodiments, local exposure of the disclosed compounds in the intestines maybe demonstrated by regulation of FXR target genes in the intestines. In some embodiments, the target genes may include: SHP, FGF19 (FGF15), IBABP, C3, OST α/β. In some embodiments, exposure of the disclosed compounds is about 40%, 50%, 55%, 60%, 65%, 70%, 75%, 80%, 85%, 90%, 95%, 99%, 99.5%, or more in the intestines. In some instances, exposure of the disclosed compounds is about 0.5%, 1%, 5%, 10%, 15%, 20%, 25%, 30%, 40%, 50%, or less in the systemic circulation. In some embodiments, the exposure of the FXR agonists in the intestinal lumen reduces the chance of side effects which results from systemic action, thereby improving the safety profile of the therapy. In additional embodiments, the disclosed compounds enhance FXR target gene expression in the intestines. In additional embodiments, the disclosed compounds further modulate gene expression in the FXR-mediated pathway, such as for example, FGF19 (FGF15) which inhibits CYP7A1 and CYP8B1 gene expression in the liver. In some instances, the disclosed compounds enhance gene expression in the FXR-mediated pathway. In other instances, the disclosed compounds reduce or inhibit gene expression in the FXR-mediated pathway. In some instances, enhancing is about 1%, 5%, 10%, 15%, 20%, 25%, 30%, 40%, 50%, 60%, 70%, 80%, 90%, 100%, 200%, 300%, 500%, 1,000%, 5,000%, 10,000%, 50,000%, 100,000%, 500,000% or higher in gene expression in the intestines, liver, kidney, or other tissues relative to the gene expression in the absence of the disclosed compound. In some cases, reducing is about 100%, 90%, 80%, 70%, 60%, 50%, 40%, 30%, 25%, 20%, 15%, 10%, 5%, 1%, or less in gene expression in the intestines, liver, kidney, or other tissues relative to the gene expression in the absence of the disclosed compound.

In some embodiments, the method substantially enhances FXR target gene expression in the intestines while minimizing systemic plasma levels of the delivered compound. In some embodiments, the method substantially enhances FXR target gene expression in the intestines and the liver while minimizing systemic plasma levels of the delivered compound. In some embodiments, the method substantially enhances FXR target gene expression in the intestines while not substantially enhancing FXR target gene expression in the liver or kidney, and while minimizing systemic plasma levels. In some embodiments, the method substantially enhances FXR target gene expression in the intestines and the liver and provides sustained systemic plasma levels of the delivered compound.

In some embodiments, metabolic disorder refers to any disorder that involves an alteration in the normal metabolism of carbohydrates, lipids, proteins, nucleic acids or a combination thereof. In some instances, a metabolic disorder is associated with either a deficiency or excess in a metabolic pathway resulting in an imbalance in metabolism of nucleic acids, proteins, lipids, and/or carbohydrates. Factors affecting metabolism include, but are not limited to, the endocrine (hormonal) control system (e.g., the insulin pathway, the enteroendocrine hormones including GLP-1, oxyntomodulin, PYY or the like), or the neural control system (e.g., GLP-1 in the brain). Exemplary metabolic disorders include, but are not limited to, diabetes, insulin resistance, dyslipidemia, liver disease, inflammation related intestinal conditions, cell proliferative disorders, or the like.

### Diabetes Mellitus and Diabetes-Related Conditions or Disorders

In some embodiments, disclosed herein are methods of treating a subject having diabetes mellitus or diabetes-related condition or disorder with administration of a FXR agonist described herein. In some instances, diabetes is type II diabetes or non-insulin-dependent diabetes mellitus (NIDDM). In some instances, diabetes-related conditions or disorders include obesity, impaired glucose tolerance, dyslipidemia, and insulin resistance. In some instances, diabetes-related conditions or disorders further include secondary complications such as atherosclerosis, stroke, fatty liver disease, blindness, gallbladder disease, or polycystic ovary disease. In some cases, a FXR agonist is administered for the treatment of type II diabetes, obesity, impaired glucose tolerance, dyslipidemia, insulin resistance, or secondary complications such as atherosclerosis, stroke, fatty liver disease, blindness, gallbladder disease, or polycystic ovary disease.

In some embodiments, a diabetic subject (e.g., a type II diabetic subject) is further characterized with a body mass index (BMI) of 25 or greater, 30 or greater, 35 or greater, 40 or greater, such as a BMI of 25 to 29, 30 to 34, or 35 to 40.

In some examples, a FXR agonist described herein reduces or prevents weight gain in a subject. In some instances, the weight gain is diet-induced weight gain. In other instances, the weight gain is non diet-related, such as familial/genetic obesity or obesity resulting from medication. In some examples, such methods reduce or prevent weight gain in the subject by at least 5%, at least 10%, at least 15%, at least 20%, at least 30%, at least 40%, at least 50%, or more. In some instances, weight gain is reduced or prevented by about 5% to about 50%, by about 5% to about 25%, by about 10% to about 20%, or by about 10% to about 30%. In some cases, the reduction or prevention of weight gain is relative to the reduction or prevention of weight gain observed in a subject not treated with the FXR agonist.

Similarly, in some cases, the FXR agonist reduces the BMI of a subject. In some examples, such methods reduce the BMI of a subject by at least 5%, at least 10%, at least 15%, at least 20%, at least 25%, at least 30%, or more, relative to a subject not treated with the FXR agonist. In some instances, the subject is overweight but not obese. In other instances, the subject is neither overweight nor obese.

In some instances, administration of a FXR agonist results in a decrease in the amount of serum lipids. In some examples, the decrease in the amount of serum lipids is by at least 5%, at least 10%, at least 15%, at least 20%, at least 30%, at least 50%, at least 60%, at least 70%, at least 75%, or more. In some cases, the decrease in the amount of serum lipids is by about 5% to about 50%, by about 5% to about 25%, by about 10% to about 20%, by about 10% to about 70%, or by about 10% to about 30%. In some cases, the decrease in the amount of serum lipids is relative to the amount of serum lipids observed in a subject not treated with the FXR agonist.

In some examples, administration of a FXR agonist results in a decrease in triglyceride (e.g., hepatic triglyceride) level. In some instances, the decrease in triglyceride (e.g., hepatic triglyceride) level is by at least 5%, at least 10%, at least 15%, at least 20%, at least 30%, at least 50%, at least 60%, at least 70%, at least 75%, or more. In some instances, the decrease in triglyceride (e.g., hepatic triglyceride) level is by about 5% to about 50%, by about 5% to about 25%, by about 10% to about 20%, by about 10% to about 70%, or by about 10% to about 30%. In some cases, the decrease in triglyceride (e.g., hepatic triglyceride) level is relative to the triglyceride (e.g., hepatic triglyceride) level observed in a subject not treated with the FXR agonist.

In some examples, administration of a FXR agonist results in an increased insulin sensitivity to insulin in the liver. In some instances, the increase in insulin sensitivity is by at least 5%, at least 10%, at least 15%, at least 20%, at least 30%, at least 40%, at least 50%, or more. In some cases, the increase in insulin sensitivity is by about 5% to about 50%, by about 5% to about 25%, by about 10% to about 20%, or by about 10% to about 30%. In some cases, the increase in insulin sensitivity is relative to sensitivity observed in a subject not treated with the FXR agonist.

In some embodiments, administration of a FXR agonist results in a decrease in the amount of serum insulin in the subject. In some examples, the decrease in serum insulin is by at least 5%, at least 10%, at least 15%, at least 20%, at least 30%, at least 50%, at least 60%, at least 70%, at least 75%, or more. In some instances, serum insulin is decreased by about 5% to about 50%, by about 5% to about 25%, by about 10% to about 20%, by about 10% to about 70%, or by about 10% to about 30%. In some cases, the decrease in serum insulin level is relative to levels observed in a subject not treated with the FXR agonist.

In some embodiments, administration of a FXR agonist results in a decrease in the amount of serum glucose in the subject. In some examples, the decrease in serum glucose is by at least 5%, at least 10%, at least 15%, at least 20%, at least 30%, at least 50%, at least 60%, at least 70%, at least 75%, or more. In some instances, serum glucose is decreased by about 5% to about 50%, by about 5% to about 25%, by about 10% to about 20%, by about 10% to about 70%, or by about 10% to about 30%. In some cases, the decrease in serum glucose level is relative to levels observed in a subject not treated with the FXR agonist.

In some examples, a FXR agonist described herein increases browning of white adipose tissue in a subject. In some examples, the rate of increase of browning of white adipose tissue in the subject is by at least 5%, at least 10%, at least 15%, at least 20%, at least 30%, at least 40%, at least 50%, or more, relative to a subject not treated with the FXR agonist.

In some embodiments, administration of a FXR agonist does not result in substantial change in food intake and/or fat consumption in the subject. In some instances, food intake and/or fat consumption is reduced, such as by less than 15%, less than 10%, or less than 5%. In some embodiments, no substantial change in appetite in the subject results. In other embodiments, reduction in appetite is minimal as reported by the subject.

In some embodiments, administration of a FXR agonist results in an increase in the metabolic rate in the subject. In some instances, the FXR agonist increases the metabolic rate in a subject. In some cases, the metabolic rate in the subject is increased by at least 5%, at least 10%, at least 15%, at least 20%, at least 30%, at least 40%, at least 50%, at least 60%, at least 70%, at least 75%, or more. In some instances, the metabolic rate is increased by about 5% to about 50%, by about 5% to about 25%, by about 10% to about 20%, by about 10% to about 70%, or by about 10% to about 30%). In some cases, the increase in metabolic rate is relative to the rate observed in a subject not treated with the FXR agonist.

In some embodiments, the increase in metabolism results from enhanced oxidative phosphorylation in the subject, which in turn leads to increased energy expenditure in tissues (such as BAT). In such instances, the FXR agonist helps to increase the activity of BAT. In some examples, the activity of BAT is increased by at least 5%, at least 10%, at least 15%, at least 20%, at least 30%, at least 50%, at least 60%, at least 70%, at least 75%, or more. In some instances, the activity of BAT is increased by about 5% to about 50%, by about 5% to about 25%, by about 10% to about 20%, by about 10% to about 70%, or by about 10% to about 30%. In some cases, the increase in BAT activity is relative to the activity of BAT observed in a subject not treated with the FXR agonist.

Alcoholic and Non-Alcoholic Liver Disease or Condition

Disclosed herein include methods of preventing and/or treating alcoholic or non-alcoholic liver diseases or conditions. Exemplary alcoholic or non-alcoholic liver diseases or conditions include, but are not limited to cholestasis, cirrhosis, steatosis, alcoholic hepatitis, non-alcoholic steatohepatitis (NASH), non-alcoholic fatty liver disease (NAFLD), primary sclerosing cholangitis (PSC), elevated liver enzymes, and elevated triglyceride levels. In some embodiments, a FXR agonist is used in the prevention or treatment of alcoholic or non-alcoholic liver diseases. In some embodiments, a FXR agonist is used in the prevention or treatment of cholestasis, cirrhosis, steatosis, alcoholic hepatitis, non-alcoholic steatohepatitis (NASH), non-alcoholic fatty liver disease (NAFLD), or primary sclerosing cholangitis (PSC).

### Cholestasis

In some embodiments, a FXR agonist disclosed herein is used in the treatment of cholestasis in a subject. Cholestasis, an impairment or cessation in the flow of bile, which in some cases, causes hepatotoxicity due to the buildup of bile acids and other toxins in the liver. In some instances, cholestasis is a component of many liver diseases, including cholelithiasis, cholestasis of pregnancy, primary biliary cirrhosis (PBC), and primary sclerosing cholangitis (PSC). In some instances, the obstruction is due to gallstone, biliary trauma, drugs, one or more additional liver diseases, or to cancer. In some cases, the enterohepatic circulation of bile acids enables the absorption of fats and fat-soluble vitamins from the intestine and allows the elimination of cholesterol, toxins, and metabolic by-products such as bilirubin from the liver. In some cases, activation of FXR induces expression of the canalicular bile transporters BSEP (ABCB11) and multidrug resistance-related protein 2 (MRP2; ABCC2, cMOAT), and represses genes involved in bile acid biosynthesis, such as for example sterol 12α-hydroxylase (CYP8B1) and CYP7A1.

In some examples, the FXR agonist reduces cholestasis in the subject by at least 5%, at least 10%, at least 15%, at least 20%, at least 30%, at least 40%, at least 50%, or more. In some cases, cholestasis is reduced by about 5% to about 50%, by about 5% to about 25%, by about 10% to about 20%, or by about 10% to about 30%. In some instances, the level of cholestasis is relative to the level of cholestasis in a subject not treated with the FXR agonist.

### Primary Biliary Cirrhosis and Cirrhosis

In some embodiments, a FXR agonist disclosed herein is used in the treatment of primary biliary cirrhosis (PBC) in a subject. PBC is a liver disease that primarily results from autoimmune destruction of the bile ducts that transport bile acids (BAs) out of the liver, resulting in cholestasis. As PBC progresses, persistent toxic buildup of BAs causes progressive liver damage. Chronic inflammation and fibrosis can advance to cirrhosis. In some examples, the FXR agonist reduces PBC in the subject by at least 5%, at least 10%, at least 15%, at least 20%, at least 30%, at least 40%, at least 50%, or more. In some cases, PBC is reduced by about 5% to about 50%, by about 5% to about 25%, by about 10% to about 20%, or by about 10% to about 30%. In some instances, the level of PBC is relative to the level of PBC in a subject not treated with the FXR agonist.

In some embodiments, a FXR agonist disclosed herein reduces cirrhosis in a subject. In some examples, the FXR agonist reduces cirrhosis in the subject by at least 5%, at least 10%, at least 15%, at least 20%, at least 30%, at least 40%, at least 50%, or more. In some cases, cirrhosis is reduced by about 5% to about 50%, by about 5% to about 25%, by about 10% to about 20%, or by about 10% to about 30%. In some instances, the level of cirrhosis is relative to the level of cirrhosis in a subject not treated with the FXR agonist.

### Non-Alcoholic Fatty Liver Disease and Non-Alcoholic Steatohepatitis

Non-alcoholic fatty liver disease (NAFLD) is associated with excessive fat in the liver (steatosis) and in some cases progresses to NASH, which is defined by the histologic hallmarks of inflammation, cell death, and fibrosis. In some instances, primary NASH is associated with insulin resistance, while secondary NASH is caused by medical or surgical conditions, or drugs such as, but not limited to, tamoxifen. In some cases, NASH progresses to advanced fibrosis, hepatocellular carcinoma, or end-stage liver disease requiring liver transplantation.

In some instances, NASH developes as a result of triglyceride (TGs) imbalance. For example, dysfunctional adipocytes secrete pro-inflammatory molecules such as cytokines and chemokines leading to insulin resistance and a failure of lipolysis suppression in the adipocytes. In some instances, this failure of lipolysis suppression leads to a release of free fatty acids (FFAs) into the circulation and uptake within the liver. In some cases, overaccumulation of FFAs in the form of triglycerides (TGs) in lipid dropletes leads to oxidative stress, mitochondrial dysfunction, and upregulation of pro-inflammatory molecules.

In some instances, activation of FXR inhibits triglyceride (TG)/fatty acid (FA) synthesis facilitated by suppressing sterol regulatory element-binding protein 1c (SREBP1c) via activation of SHP. In some cases, FXR additionally increases the clearance of TG by stimulating lipoprotein lipase (LPL) activity as well as the hepatic uptake of remnants and low-density lipoprotein by inducing syndecan 1 (SDC1) and the VLDL receptor (VLDLR).

In some embodiments, a FXR agonist disclosed herein is used in the treatment of non-alcoholic steatohepatitis (NASH). In some examples, the FXR agonist reduces NASH the subject by at least 5%, at least 10%, at least 15%, at least 20%, at least 30%, at least 40%, at least 50%, or more. In some cases, NASH is reduced by about 5% to about 50%, by about 5% to about 25%, by about 10% to about 20%, or by about 10% to about 30%. In some instances, the level of NASH is relative to the level of NASH in a subject not treated with the FXR agonist.

In some embodiments, a FXR agonist disclosed herein is used in the treatment of NAFLD. In some examples, the FXR agonist reduces NAFLD in the subject by at least 5%, at least 10%, at least 15%, at least 20%, at least 30%, at least 40%, at least 50%, or more. In some cases, NAFLD is reduced by about 5% to about 50%, by about 5% to about 25%, by about 10% to about 20%, or by about 10% to about 30%. In some instances, the level of NAFLD is relative to the level of NAFLD in a subject not treated with the FXR agonist.

### Steatosis

In some embodiments, a FXR agonist disclosed herein reduces fatty liver (steatosis) in a subject. In some examples, the FXR agonist reduces steatosis in the subject by at least 5%, at least 10%, at least 15%, at least 20%, at least 30%, at least 40%, at least 50%, or more. In some instances, steatosis is reduced by about 5% to about 50%, by about 5% to about 25%, by about 10% to about 20%, or by about 10% to about 30%. In some instances, the level of steatosis is relative to the level of steatosis in a subject not treated with the FXR agonist.

### Alcoholic Hepatitis

In some embodiments, a FXR agonist disclosed herein reduces alcoholic hepatitis in a subject. In some examples, the FXR agonist reduces alcoholic hepatitis in the subject by at least 5%, at least 10%, at least 15%, at least 20%, at least 30%, at least 40%, at least 50%, or more. In some instances, the level of alcoholic hepatitis is reduced by about 5% to about 50%, by about 5% to about 25%, by about 10% to about 20%, or by about 10% to about 30%. In some instances, the level of alcoholic hepatitis is relative to the level of alcoholic hepatitis in a subject not treated with the FXR agonist.

### Primary Sclerosing Cholangitis

In some embodiments, a FXR agonist disclosed herein is used in the treatment of primary sclerosing cholangitis (PSC). PSC is a chronic and progressive cholestatic liver disease. PSC is characterized by progressive inflammation, fibrosis, and stricture formation in liver ducts. Common symptoms include pruritus and jaundice. The disease is strongly associated with inflammatory bowel disease (IBD)—about 5% of patients with ulcerative colitis will have PSC. Up to 70% of patients with PSC also have IBD, most commonly ulcerative colitis.

### Additional Alcoholic and Non-Alcoholic Liver Diseases or Conditions

In some embodiments, a FXR agonist disclosed herein reduces liver enzymes in a subject. In some examples, the FXR agonist reduce liver enzymes (e.g., serum ALT and/or AST levels) in the subject by at least 5%, at least 10%, at least 15%, at least 20%, at least 30%, at least 40%, at least 50%, or more. In some instances, the level of liver enzymes is reduced by about 5% to about 50%, by about 5% to about 25%, by about 10% to about 20%, or by about 10% to about 30%. In some instances, the level of liver enzymes is relative to the level of liver enzymes in a subject not treated with the FXR agonist.

In some embodiments, a FXR agonist disclosed herein reduces liver triglycerides in a subject. In some examples, the FXR agonist reduces liver triglycerides in the subject by at least 5%, at least 10%, at least 15%, at least 20%, at least 30%, at least 40%, at least 50%, or more. In some instances, the level of liver triglycerides is reduced by about 5% to about 50%, by about 5% to about 25%, by about 10% to about 20%, or by about 10% to about 30%. In some instances, the level of liver triglycerides is relative to the level of liver triglycerides in a subject not treated with the FXR agonist.

### Inflammatory Intestinal Condition

Disclosed herein are methods of treating or preventing an inflammatory intestinal condition. Exemplary inflammatory conditions include necrotizing enterocolitis (NEC), gastritis, ulcerative colitis, inflammatory bowel disease, irritable bowel syndrome, pseudomembranous colitis, gastroenteritis, radiation induced enteritis, chemotherapy induced enteritis, gastro-esophageal reflux disease (GERD), peptic ulcer, non-ulcer dyspepsia (NUD), celiac disease, intestinal celiac disease, gastrointestinal complications following bariatric surgery, gastric carcinogenesis, or gastric carcinogenesis following gastric or bowel resection. In some embodiments, the inflammatory condition is NEC and the subject is a newborn or prematurely born infant. In some embodiments, the subject is enterally-fed infant or formula-fed infant.

In some embodiments, a FXR agonist disclosed herein is administered to a subject having an inflammatory intestinal condition. In some embodiments, a FXR agonist disclosed herein is administered to a subject having necrotizing enterocolitis (NEC), gastritis, ulcerative colitis, inflammatory bowel disease, irritable bowel syndrome, pseudomembranous colitis, gastroenteritis, radiation induced enteritis, chemotherapy induced enteritis, gastroesophageal reflux disease (GERD), peptic ulcer, non-ulcer dyspepsia (NUD), celiac disease, intestinal celiac disease, gastrointestinal complications following bariatric surgery, gastric carcinogenesis, or gastric carcinogenesis following gastric or bowel resection.

In some embodiments, a FXR agonist disclosed herein reduces inflammation of the intestines in a subject (such as a human). In some examples, the FXR agonist reduces intestinal inflammation in the subject by at least 5%, at least 10%, at least 15%, at least 20%, at least 30%, atleast 40%, at least 50%, or more. In some instances, intestinal inflammation is reduced by about 5% to about 50%, by about 5% to about 25%, by about 10% to about 20%, or by about 10% to about 30%. In some instances, the level of intestinal inflammation is relative to the level of intestinal inflammation in a subject not treated with the FXR agonist.

### Cell Proliferation Disease

Further disclosed herein are methods of preventing or treating cell proliferation diseases, for example, in certain types of cancer. In some embodiments, the FXR agonists disclosed herein are used in the prevention or treatment of adenocarcinomas, or a carcinoma derived from glandular tissue or in which the tumor cells form recognizable glandular structures. In some embodiments, adenocarcinomas are classified according to the predominant pattern of cell arrangement, as papillary, alveolar, or according to a particular product of the cells, as mucinous adenocarcinoma. In some instances, adenocarcinomas are observed for example, in colon, kidney, breast, cervix, esophagus, gastric, pancreas, prostate, or lung.

In some embodiments, the compounds disclosed herein are used in the prevention or treatment of a cancer of the intestine, such as colon cancer, e.g. cancer that forms in the tissues of the colon (the longest part of the large intestine), or a cancer of another part of the intestine, such as the jejunum, and/or ileum. In some instances, colon cancer is also referred to as “colorectal cancer.” In some instances, the most common type of colon cancer is colon adenocarcinoma.

In some cases, cancer progression is characterized by stages, or the extent of cancer in the body. Staging is usually based on the size of the tumor, the presence of cancer in the lymph nodes, and the presence of the cancer in a site other than the primary cancer site. Stages of colon cancer include stage I, stage n, stage in and stage IV. In some embodiments, colon adenocarcinoma is from any stage. In other embodiments, colon adenocarcinoma is a stage I cancer, a stage II cancer or a stage III cancer.

In some embodiments, a FXR agonist described herein is administered to a subject having a stage I, stage II, stage III, or stage IV cancer. In some instances, a FXR agonist described herein is administered to a subject having a stage I, stage II, or stage III colon adenocarcinoma.

In some embodiments, a FXR agonist disclosed herein further reduces the tumor burden in a subject. In some examples, the FXR agonist reduces tumor burden (such as colon tumor burden) in the subject by at least 5%, at least 10%, at least 15%, at least 20%, at least 30%, at least 40%, at least 50%, or more. In some instances, tumor burden is reduced by about 5% to about 50%, by about 5% to about 25%, by about 10% to about 20%, or by about 10% to about 30%. In some instances, the level of tumor burden is relative to the level of tumor burden in a subject not treated with the FXR agonist.

In some instances, a FXR agonist disclosed herein further reduces tumor size and/or volume in a subject. In some cases, the FXR agonist reduces tumor size and/or volume (such as a colon tumor) in the subject by at least 5%, at least 10%, at least 15%, at least 20%, at least 30%, at least 40%, at least 50%, or more. In some instances, tumor size is reduced by about 5% to about 50%, by about 5% to about 25%, by about 10% to about 20%, or by about 10% to about 30%. In some instances, the tumor size is relative to the tumor size in a subject not treated with the FXR agonist.

In additional embodiments, a FXR agonist disclosed herein reduces effects of cachexia due to a tumor in a subject. In some examples, the FXR agonist reduce the effect of cachexia (such as due to a colon tumor) in the subject by at least 5%, at least 10%, at least 15%, at least 20%, at least 30%, at least 40%, at least 50%, or more. In some instances, the effect of cachexia is reduced by about 5% to about 50%, by about 5% to about 25%, by about 10% to about 20%, or by about 10% to about 30%. In some instances, the effect of cachexia is relative to the effect of cachexia in a subject not treated with the FXR agonist.

In other embodiments, a FXR agonist disclosed herein increases survival rates of a subject with a tumor. In some cases, the FXR agonist increases the survival rate of a subject with a tumor (such as a colon cancer) in the subject by at least 5%, at least 10%, at least 15%, at least 20%, at least 30%, at least 40%, at least 50%, or more. In some instances, survival rate is increased by about 5% to about 50%, by about 5% to about 25%, by about 10% to about 20%, or by about 10% to about 30%. In some instances, the survival rate is relative to the survival rate in a subject not treated with the FXR agonist.

Compounds

Compounds described herein, including pharmaceutically acceptable salts, prodrugs, active metabolites and pharmaceutically acceptable solvates thereof, are famesoid X receptor agonists.

In one aspect, described herein is a compound of Formula (I), or a pharmaceutically acceptable salt, or solvate thereof:

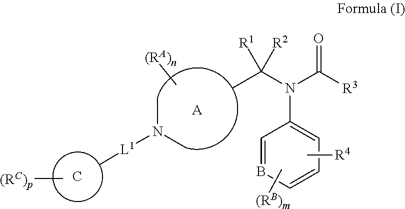

wherein,
R¹ and R² are each independently selected from H, D, F, C₁-C₄alkyl, or C₁-C₄fluoroalkyl;
or R¹ and R² are taken together with the carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NHC(═O)R¹¹, —NHC(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
L¹ is —X¹-L²- or -L²-X¹—,
   X¹ is absent, —S(═O)—, —S(═O)₂—, —C(═O)—, —OC(═O)—, —NR¹⁰C(═O)—, or —NR¹⁰S(═O)₂—;
   L² is absent or substituted or unsubstituted C₁-C₄alkylene;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
      R⁵ and R⁷ are each independently selected from H, D, CᵣC₄alkyl and C₃-C₆cycloalkyl,
      or R⁵ and R⁷ are taken together with the intervening atoms to form a double bond;
      or R⁵ and R⁷ are taken together with the intervening atoms to form an epoxide or an substituted or unsubstituted C₃-C₆cycloalkyl;
      R⁶ and R⁸ are each independently selected from H, D, C₁-C₄alkyl or C₃-C₆cycloalkyl;
   Y is —CH₂OR⁹, —C(═O)OR⁹,

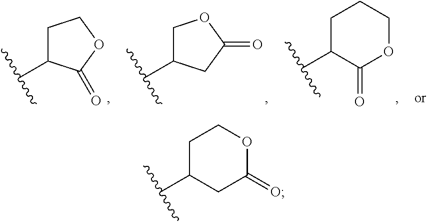 [SMILES: *C1CCOC1=O ; *C1COC(=O)C1 ; *C1CCCOC1=O ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
ring A is a monocyclic C₂-C₈heterocycloalkyl containing 1 N atom in the ring, or bicyclic C₅-C₈heterocycloalkyl;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

For any and all of the embodiments, substituents are selected from among a subset of the listed alternatives. For example, in some embodiments, R¹ and R² are each independently selected from H, D, F, C₁-C₄alkyl, or C₁-C₄fluoroalkyl. In other embodiments, R¹ and R² are each independently selected from H, D, F, CH₃, or CF₃. In some other embodiments, R¹ and R² are each independently selected from H, or D. In some other embodiments, R¹ and R² are each H.

In some embodiments, ring A is a monocyclic C₂-C₈heterocycloalkyl containing 1 N atom in the ring that is selected from azetidinyl, pyrrolidinyl, piperidinyl, or azepanyl.

In some embodiments,

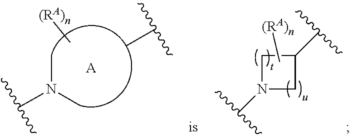

wherein, t is 1, 2, or 3; u is 1, 2, or 3.

In some embodiments,

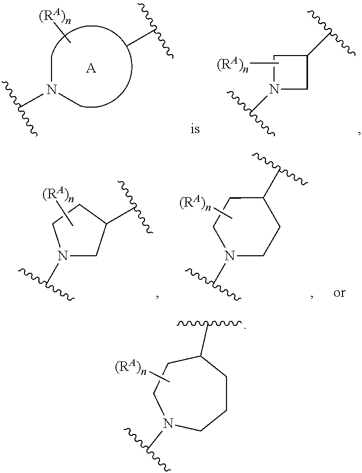

In some embodiments, ring A is a monocyclic C₂-C₈heterocycloalkyl containing 1 N atom in the ring that is selected from a β-lactam, γ-lactam, δ-lactam or ε-lactam.

In some embodiments, ring A is a bicyclic C₅-C₈heterocycloalkyl that is a fused bicyclic C₅-C₈heterocycloalkyl, bridged bicyclic C₅-C₈heterocycloalkyl, or spiro bicyclic C₅-C₈heterocycloalkyl.

In some embodiments,

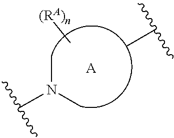

is a bridged bicyclic C₅-C₈heterocycloalkyl that is

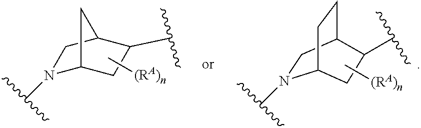

In some embodiments,

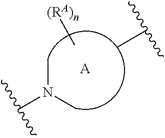

is a spiro bicyclic C₅-C₈heterocycloalkyl that is

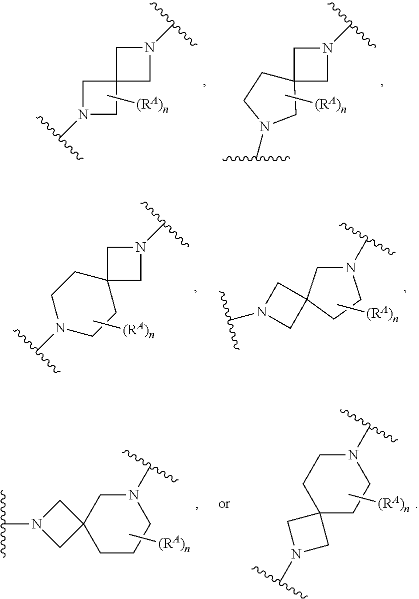

In some embodiments, X¹ is absent, —S(═O)—, —S(═O)₂—, —C(═O)—, —OC(═O)—, —NR¹⁰C(═O)—, or —NR¹⁰S(═O)₂—; L² is absent or —CH₂—.

In some embodiments, L¹ is absent, —CH₂—, —S(═O)₂—, or —C(═O)—.

In some embodiments, R⁴ is

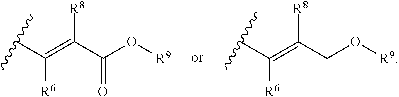

In some embodiments, R⁴ is

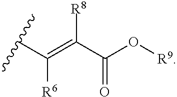

In some embodiments, R⁴ is

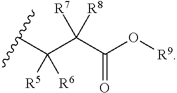

In some embodiments, R⁴ is

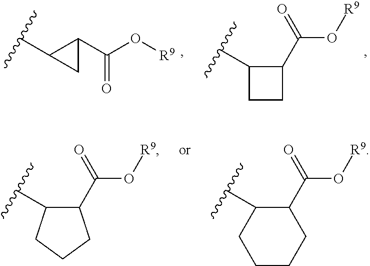

In some embodiments, R⁴ is -L³-Y; L³ is —CH₂—; Y is

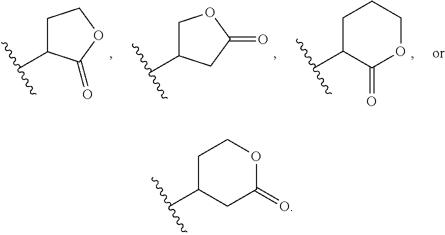 [SMILES: *C1CCOC1=O ; *C1COC(=O)C1 ; *C1CCCOC1=O ; *C1CCOC(=O)C1]

In some embodiments, ring C is monocyclic carbocycle or bicyclic carbocycle.

In some embodiments, ring C is monocyclic carbocycle selected from cyclopropyl, cyclobutyl, cyclopentyl, cyclohexyl, and phenyl.

In some embodiments, ring C is phenyl.

In some embodiments, ring C is bicyclic carbocycle selected from indanyl, indenyl, and naphthyl.

In some embodiments, ring C is monocyclic heterocycle or bicyclic heterocycle.

In some embodiments, ring C is monocyclic heterocycle or bicyclic heterocycle selected from pyridinyl, pyrazolyl, pyrrolyl, imidazolyl, oxazolyl, isoxazolyl, thiazolyl, isothiazolyl, triazolyl, pyrimidinyl, pyrazinyl, triazinyl, benzimidazolyl, indolyl, quinolinyl, indazolyl, purinyl, quinoxalinyl, and acridinyl.

In some embodiments,

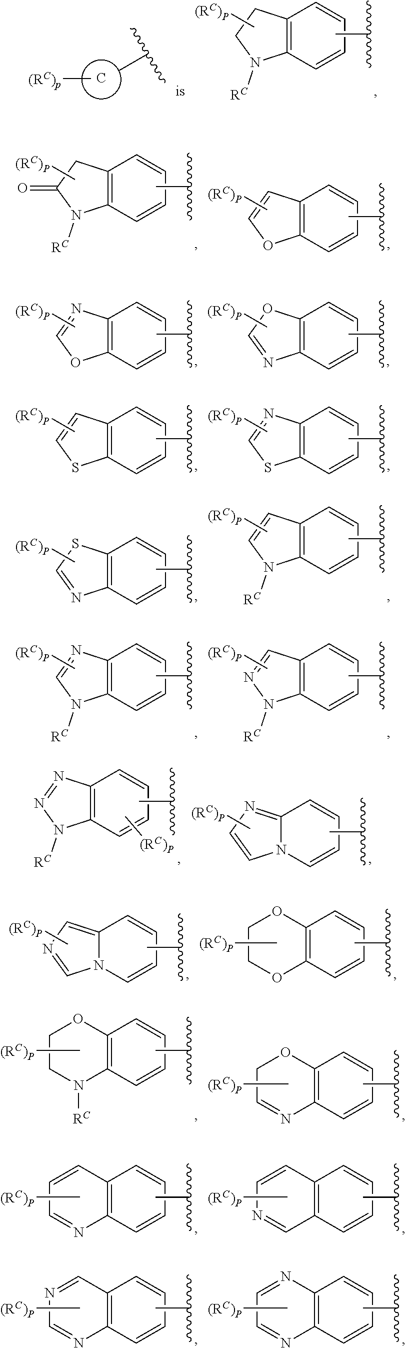

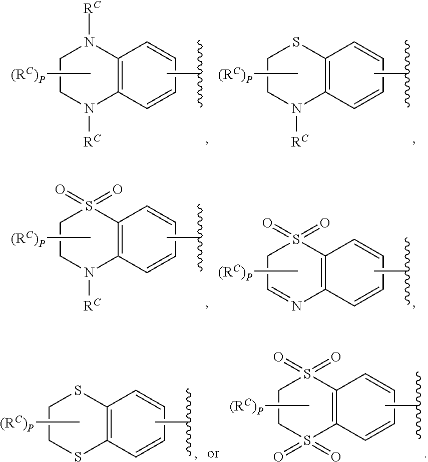

In some embodiments, ring C is monocyclic heteroaryl selected from furanyl, thienyl, pyridinyl, pyrazolyl, pyrrolyl, imidazolyl, oxazolyl, isoxazolyl, thiazolyl, isothiazolyl, triazolyl, pyrimidinyl, pyrazinyl, and triazinyl.

In some embodiments, ring C is a monocyclic 6-membered heteroaryl containing 1-3 N atoms.

In some embodiments,

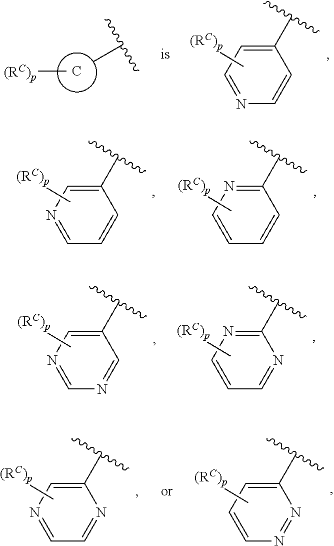

In some embodiments, ring C is a monocyclic 5-membered C₁-C₄heteroaryl containing 1-4 N atoms, O or 1 O or S atom.

In some embodiments, ring C is pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments,

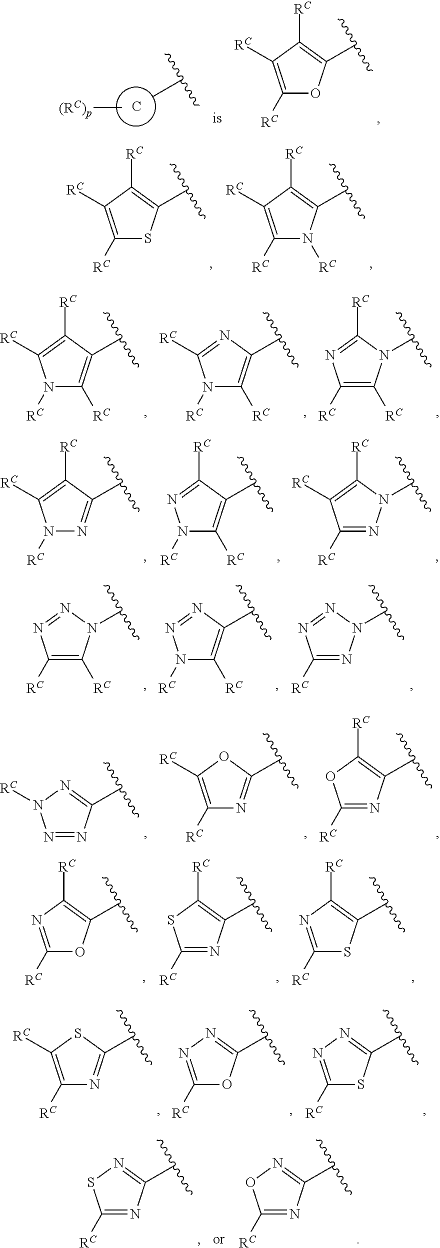

In some embodiments, ring C is monocyclic heterocycle selected from pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, and 1,2,3,6-tetrahydropyridinyl.

In some embodiments, ring C is a monocyclic C₂-C₈heterocycloalkyl containing at least 1 N atom in the ring.

In some embodiments, ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, and azepanyl.

In some embodiments, R³ is selected from substituted or unsubstituted C₁-C₁₀alkyl, substituted or unsubstituted C₃-C₁₀cycloalkyl, or substituted or unsubstituted aryl, wherein if R³ is substituted then R³ is substituted with one or more R¹² groups.

In some embodiments, R³ is selected from methyl, ethyl, propyl, iso-propyl, n-butyl, iso-butyl, tert-butyl, substituted or unsubstituted cyclopropyl, substituted or unsubstituted cyclobutyl, substituted or unsubstituted cyclopentyl, substituted or unsubstituted cyclohexyl, substituted or unsubstituted phenyl,

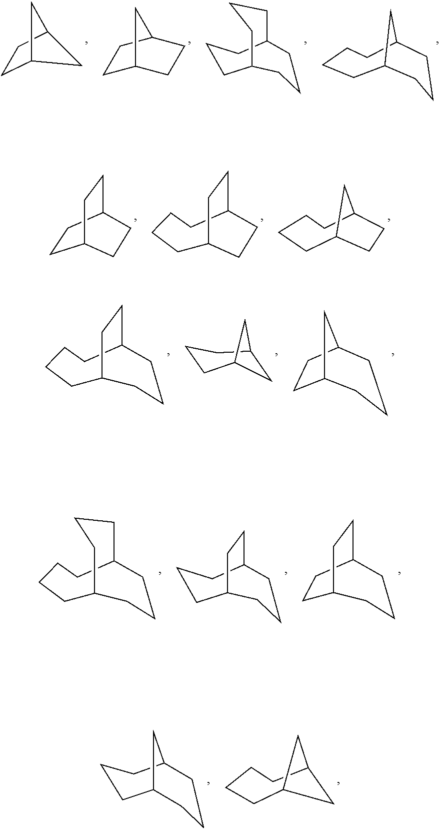

and adamantyl. In some embodiments, R³ is selected from methyl, ethyl, propyl, iso-propyl, n-butyl, iso-butyl, tert-butyl, substituted or unsubstituted cyclopropyl, substituted or unsubstituted cyclobutyl, substituted or unsubstituted cyclopentyl, substituted or unsubstituted cyclohexyl, substituted or unsubstituted phenyl,

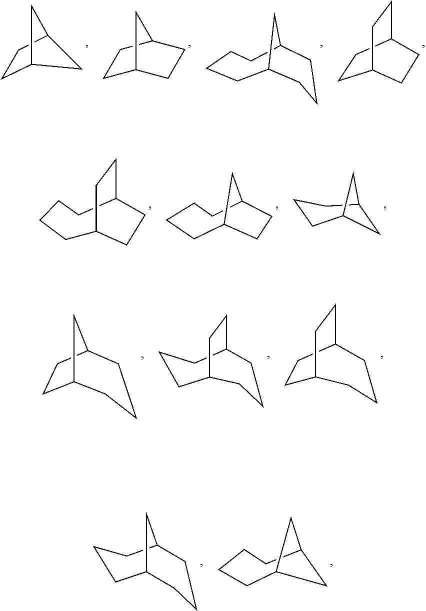

and adamantyl.

In another aspect, described herein is a compound that has the structure of Formula (II), or a pharmaceutically acceptable salt or solvate thereof:

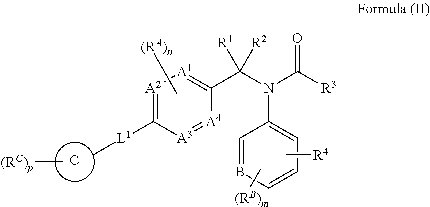

wherein,
A¹ is CR^{A};
or A¹ is N if at least one of A², A³, or A⁴ is N;
A² is CR^{A} or N;
A³ is CR^{A} or N;
A⁴ is CR^{A};
or A⁴ is N if at least one of A¹, A², or A³ is N
provided that at least one of A¹, A², A³, or A⁴ is N;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
R¹ and R² are each independently selected from H, D, F, C₁-C₄alkyl, or C₁-C₄fluoroalkyl;
or R¹ and R² are taken together with die carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NHC(═O)R¹¹, —NHC(═O)OR¹¹, unsubstituted or substituted C₁-C₁₀alkyl, unsubstituted or substituted C₁-C₁₀fluoroalkyl, unsubstituted or substituted C₂-thioalkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂—, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, unsubstituted or substituted C₂-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
   R⁵ and R⁷ are each independently selected from H, D, C₁-C₄alkyl and C₃-C₆cycloalkyl;
   or R⁵ and R⁷ are taken together with the intervening atoms to form a double bond;
   or R⁵ and R⁷ are taken together with the intervening atoms to form an epoxide or an substituted or unsubstituted C₃-C₆cycloalkyl;
   R⁶ and R⁸ are each independently selected from H, D, C₁-C₄alkyl or C₃-C₆cycloalkyl;
   Y is —CH₂OR⁹, —C(═O)OR⁹,

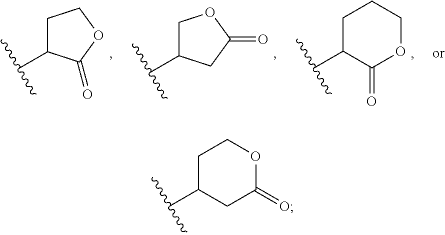 [SMILES: *C1CCOC1=O ; *C1COC(=O)C1 ; *C1CCCOC1=O ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
L¹ is —X¹-L²- or -L²-X¹—;
   X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
   L² is absent or substituted or unsubstituted C₁-C₄alkylene;
B is CR^{B}, or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
ring C is monocyclic carbocycle, bicyclic carbocycle, monocyclic N-containing heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl, and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

In some embodiments, A¹ is CR^{A}; A² is N; A³ is CR^{A} or N; A⁴ is CR^{A} or N.

In some embodiments, A¹ is CR^{A}; A² is N; A³ is CR^{A}; A⁴ is CR^{A}.

In some embodiments, A¹ is CR^{A}; A² is N; A³ is N; A⁴ is CR^{A}.

In some embodiments, A¹ is CR^{A}; A² is N; A³ is CR^{A}; A⁴ is N.

In some embodiments, A¹ is N; A² is N; A³ is CR^{A}; A⁴ is CR^{A}.

In some embodiments, A¹ is N; A² is CR^{A}; A³ is CR^{A}; A⁴ is N.

In some embodiments,

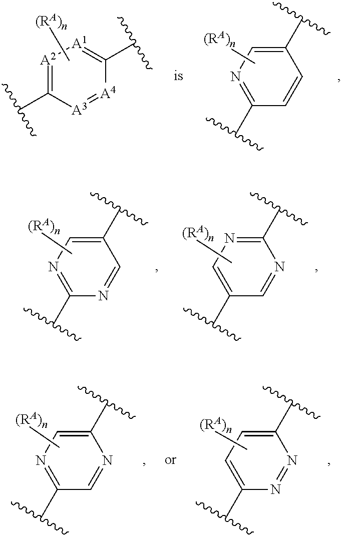

In some embodiments, L¹ is —X¹-L²-, —V—X¹—; X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, or —NR¹⁰—; L² is absent or —CH₂—.

In some embodiments, L¹ is absent, —O—, —S—, —S—CH₂—, —CH₂—S—, —CH₂—, —CH═CH—, —C≡C—, —C(═O)—, —C(═O)O—, —OC(═O)—, —C(═O)NR¹⁰—, —NR¹⁰C(═O)—, —NR¹⁰C(═O)—, —NR¹⁰—, —NR¹⁰—CH₂—, or —CH₂—NR¹⁰—.

In some embodiments, L¹ is absent, —O—, —S—, —S—CH₂—, —CH₂—S—, —CH₂—, —CH═CH—, —C≡C—, —NR¹⁰—, —NR¹⁰—CH₂—, or —CH₂—NR¹⁰—. In some embodiments, L¹ is absent, —O—, —S—, —CH₂—, —CH═CH—, —C≡C—, or —NR¹⁰—. In some embodiments, L¹ is absent, —O—, —S—, —CH₂—, —CH═CH—, —C≡C—, or —NR¹⁰—.

In some embodiments, R⁴ is

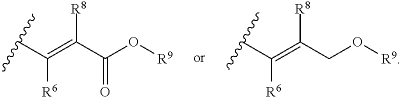

In some embodiments, R⁴ is

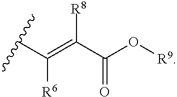

In some embodiments, R⁴ is

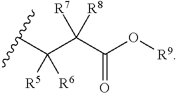

In some embodiments, R⁴

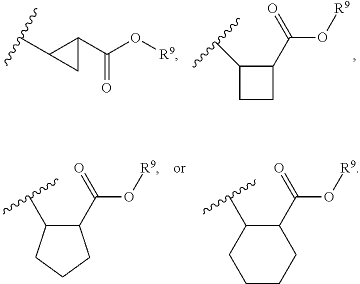

In some embodiments, R⁴ is

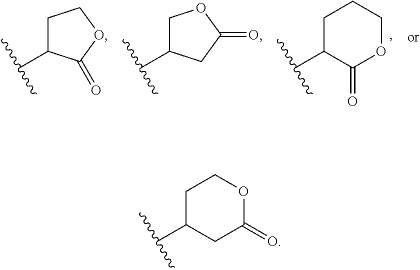 [SMILES: *C1CCOC1=O ; *C1COC(=O)C1 ; *C1CCCOC1=O ; "," ; "or" ; *C1CCOC(=O)C1]

In some embodiments, ring C is monocyclic carbocycle or bicyclic carbocycle.

In some embodiments, ring C is monocyclic carbocycle selected from cyclopropyl, cyclobutyl, cyclopentyl, cyclohexyl, and phenyl.

In some embodiments, ring C is phenyl.

In some embodiments, ring C is bicyclic carbocycle selected from indanyl, indenyl, and naphthyl.

In some embodiments, ring C is monocyclic heterocycle or bicyclic heterocycle.

In some embodiments, ring C is monocyclic heterocycle or bicyclic heterocycle selected from pyridinyl, pyrazolyl, pyrrolyl, imidazolyl, oxazolyl, isoxazolyl, thiazolyl, isothiazolyl, triazolyl, pyrimidinyl, pyrazinyl, triazinyl, benzimidazolyl, indolyl, quinolinyl, indazolyl, purinyl, quinoxalinyl, and acridinyl.

In some embodiments,

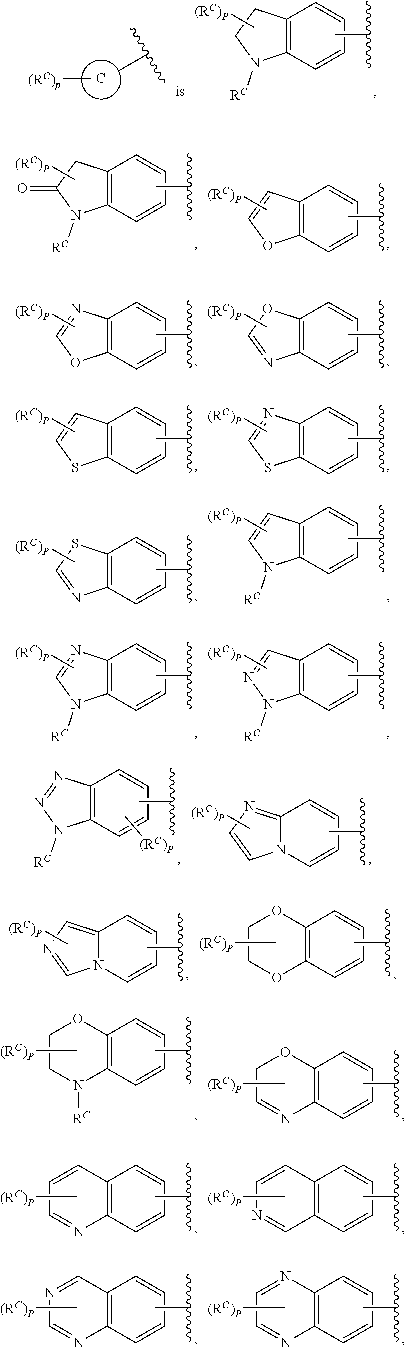

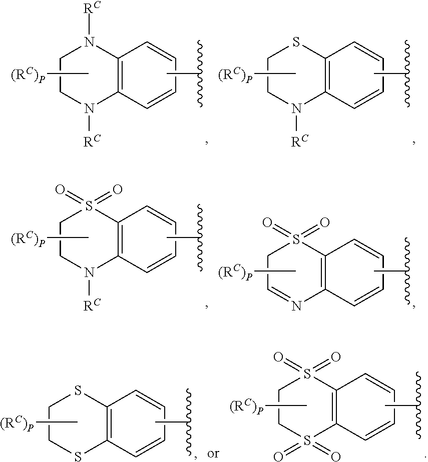

In some embodiments, ring C is monocyclic heteroaryl selected from furanyl, thienyl, pyridinyl, pyrazolyl, pyrrolyl, imidazolyl, oxazolyl, isoxazolyl, thiazolyl, isothiazolyl, triazolyl, pyrimidinyl, pyrazinyl, and triazinyl.

In some embodiments, ring C is a monocyclic 6-membered heteroaryl containing 1-3 N atoms.

In some embodiments,

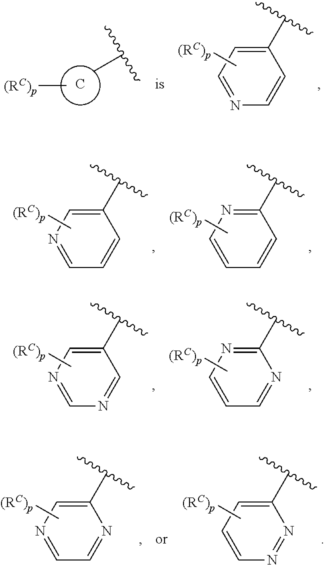

In some embodiments, ring C is a monocyclic 5-membered C₁-C₄heteroaryl containing 1-4 N atoms, 0 or 1 O or S atom.

In some embodiments, ring C is pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments,

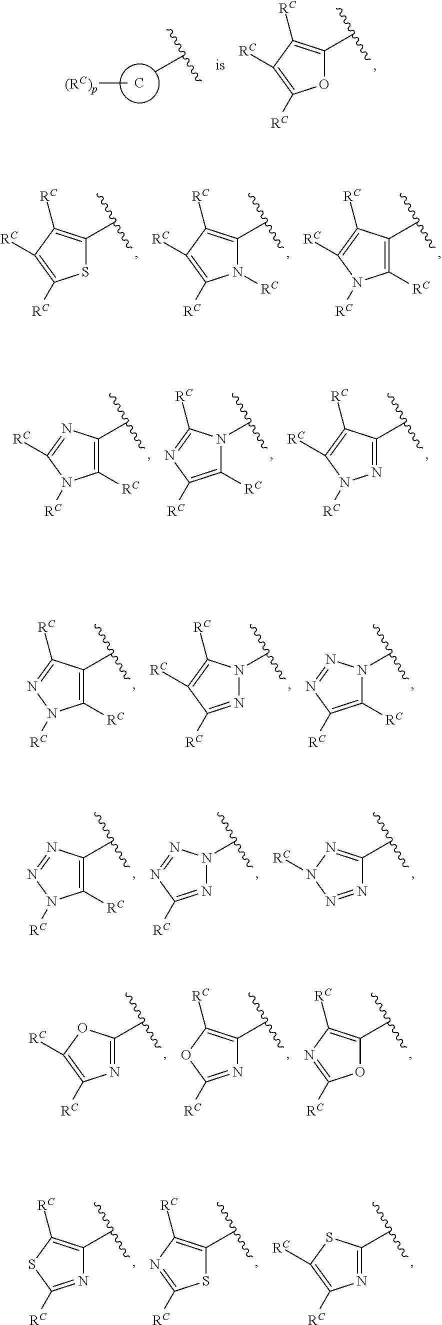

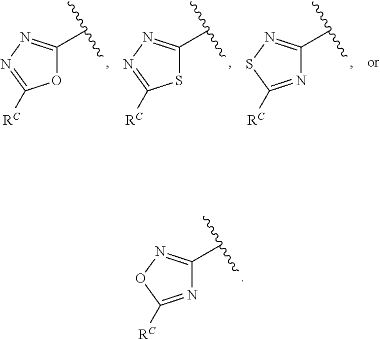

In some embodiments, ring C is monocyclic heterocycle selected from pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, and 1,2,3,6-tetrahydropyridinyl.

In some embodiments, ring C is a monocyclic C₂-C₈heterocycloalkyl containing at least 1 N atom in the ring.

In some embodiments, ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, and azepanyl.

In some embodiments,

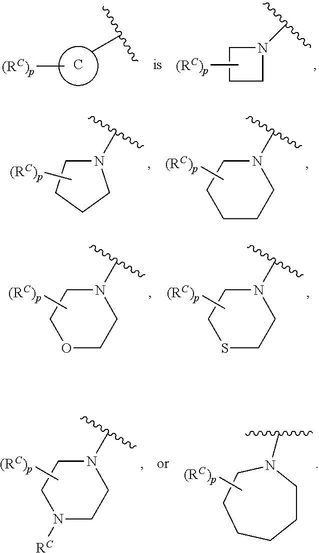

In some embodiments, the compound of Formula (I) has the structure of Formula (III), or a pharmaceutically acceptable salt or solvate thereof:

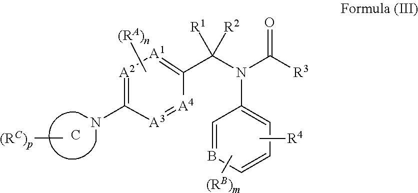

wherein,

ring C is a 5-membered N-containing heteroaryl or a N-containing C₂-C₈heterocycloalkyl.

In some embodiments, ring C is a 5-membered N-containing heteroaryl containing 1-4 N atoms.

In some embodiments,

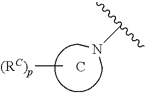

is a monocyclic 5-membered C₁-C₄heteroarylene containing 1-4 N atoms that has the structure

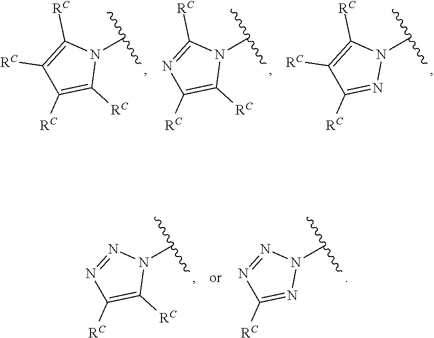

In some embodiments, ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, and azepanyl.

In some embodiments,

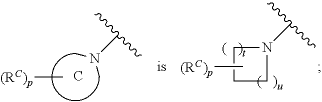

wherein, t is 1, 2, or 3; u is 1, 2, or 3.

In some embodiments,

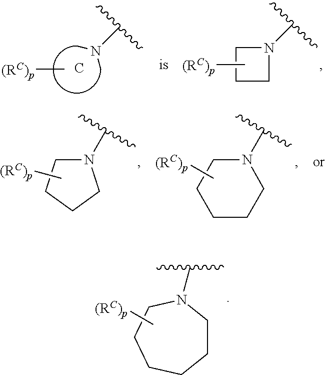

In some embodiments, ring C is a monocyclic C₂-C₈heterocycloalkyl containing 1 N atom in the ring that is selected from a β-lactam, γ-lactam, δ-lactam or ε-lactam.

In some embodiments, ring C is a bicyclic C₅-C₈heterocycloalkyl that is a fused bicyclic C₅-C₈heterocycloalkyl, bridged bicyclic C₅-C₈heterocycloalkyl, or spiro bicyclic C₅-C₈heterocycloalkyl.

In some embodiments,

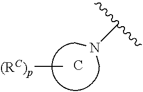

is a bridged bicyclic C₅-C₈heterocycloalkyl that is

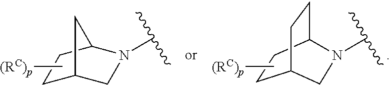

In some embodiments, ring C is a spiro bicyclic C₅-C₈heterocycloalkyl such that

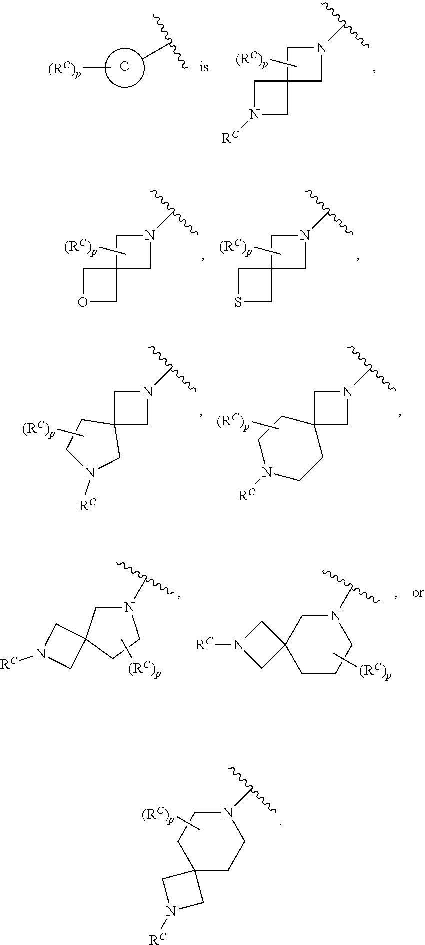

In some embodiments, R³ is selected from substituted or unsubstituted C₁-C₁₀alkyl, substituted or unsubstituted C₃-C₁₀cycloalkyl, or substituted or unsubstituted aryl, wherein if R³ is substituted then R³ is substituted with one or more R¹² groups.

In some embodiments, R³ is selected from methyl, ethyl, propyl, iso-propyl, n-butyl, iso-butyl, tert-butyl, substituted or unsubstituted cyclopropyl, substituted or unsubstituted cyclobutyl, substituted or unsubstituted cyclopentyl, substituted or unsubstituted cyclohexyl, substituted or unsubstituted phenyl,

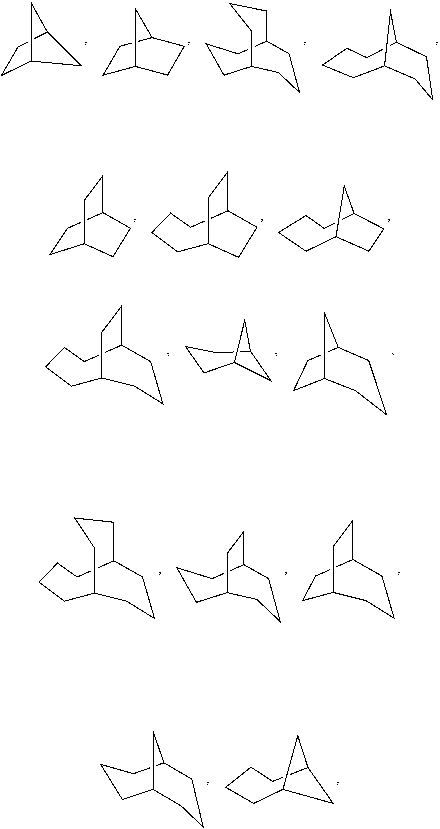

and adamantyl.

In another aspect, described herein is a compound that has the structure of Formula (IV), or a pharmaceutically acceptable salt or solvate thereof:

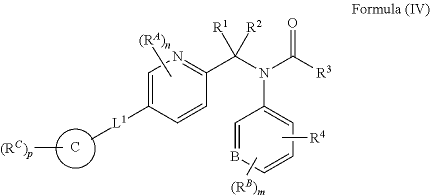

wherein,
R¹ and R² are each independently selected from H, D, F, C₁-C₄alkyl, or C₁-C₄fluoroalkyl;
or R¹ and R² are taken together with die carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NHC(═O)R¹¹, —NHC(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂₋, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
   R⁵ and R⁷ are each independently selected from H, D, C₁-C₄alkyl and C₃-C₆cycloalkyl;
   or R⁵ and R⁷ are taken together with the intervening atoms to form a double bond;
   or R⁵ and R⁷ are taken together with the intervening atoms to form an epoxide or an substituted or unsubstituted C₃-C₆cycloalkyl;
   R⁶ and R⁸ are each independently selected from H, D, C₁-C₄alkyl or C₃-C₆cycloalkyl;
   Y is —CH₂OR⁹,

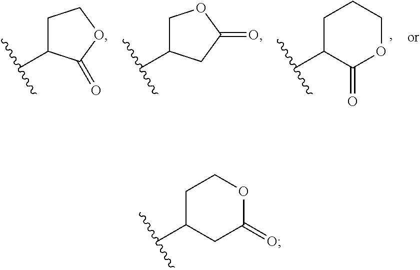 [SMILES: *C1CCOC1=O ; *C1COC(=O)C1 ; *C1CCCOC1=O ; "," ; "or" ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
L¹ is —X¹-L²-, -L²-X¹—;
   X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
   L² is absent or substituted or unsubstituted C₁-C₄alkylene;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
ring C is monocyclic carbocycle, bicyclic carbocycle, monocyclic N-containing heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl, and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4;
provided that the compound is not methyl (E)-3-(3-(N-((5-(4-(dimethylamino)phenyl)pyridin-2-yl)methyl)cyclohexanecarboxamido)phenyl)acrylate

In some embodiments, L¹ is —X¹-L²-, —V—X¹—, X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, or —NR¹⁰—; L² is absent or —CH₂—.

In some embodiments, L¹ is absent, —O—, —S—, —S—CH₂—, —CH₂—S—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, —NR¹⁰—, —NR¹⁰—CH₂—, or —CH₂—NR¹⁰—.

In some embodiments, R⁴ is

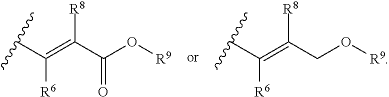

In some embodiments, R⁴ is

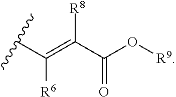

In some embodiments, R⁴ is

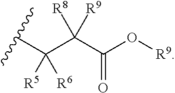

In some embodiments, R⁴ is

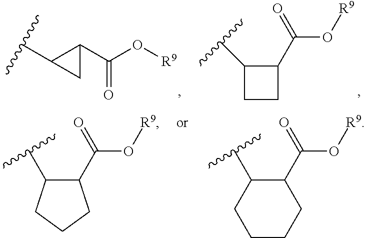

In some embodiments, R⁴ is -L³-Y; L³ is —CH₂—; Y is

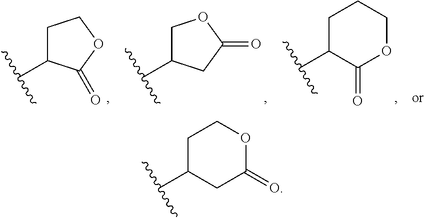 [SMILES: *C1CCOC1=O ; *C1COC(=O)C1 ; *C1CCCOC1=O ; *C1CCOC(=O)C1]

In some embodiments, L¹ is absent, —O—, —S—, —S—CH₂—, —CH₂—S—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, —NR¹⁰—, —NR¹⁰—CH₂—, or —CH₂—NR¹⁰—.

In some embodiments, ring C is monocyclic carbocycle or bicyclic carbocycle.

In some embodiments, ring C is monocyclic carbocycle selected from cyclopropyl, cyclobutyl, cyclopentyl, cyclohexyl, and phenyl.

In some embodiments, ring C is phenyl.

In some embodiments, ring C is bicyclic carbocycle selected from indanyl, indenyl, and naphthyl.

In some embodiments, ring C is monocyclic heterocycle or bicyclic heterocycle.

In some embodiments, ring C is monocyclic heterocycle or bicyclic heterocycle selected from pyridinyl, pyrazolyl, pyrrolyl, imidazolyl, oxazolyl, isoxazolyl, thiazolyl, isothiazolyl, triazolyl, pyrimidinyl, pyrazinyl, triazinyl, benzimidazolyl, indolyl, quinolinyl, indazolyl, purinyl, quinoxalinyl, and acridinyl.

In some embodiments,

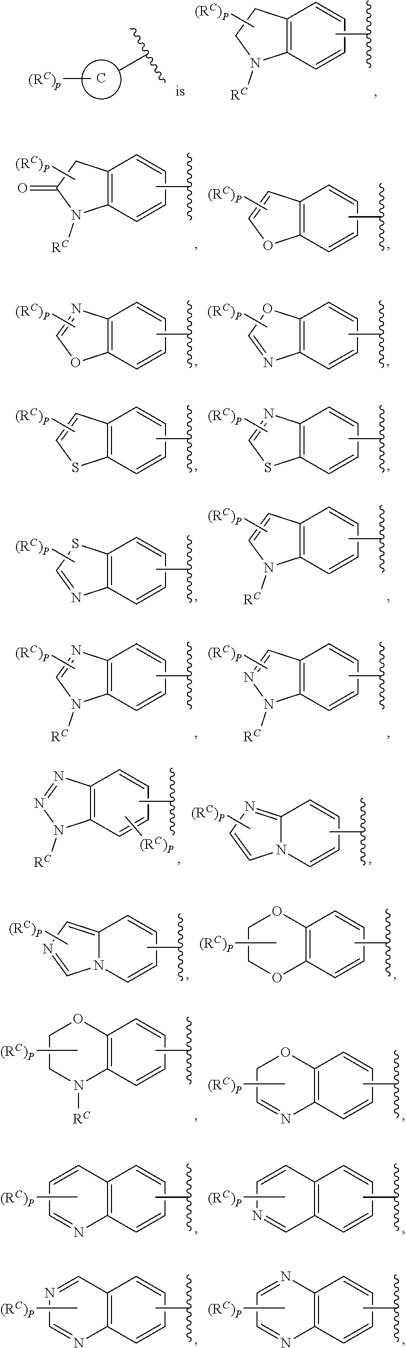

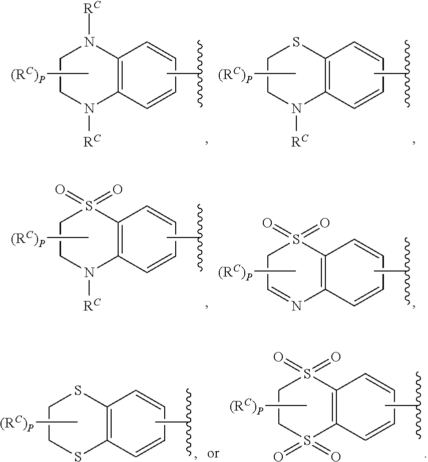

In some embodiments, ring C is monocyclic heteroaryl selected from furanyl, thienyl, pyridinyl, pyrazolyl, pyrrolyl, imidazolyl, oxazolyl, isoxazolyl, thiazolyl, isothiazolyl, triazolyl, pyrimidinyl, pyrazinyl, and triazinyl.

In some embodiments, ring C is a monocyclic 6-membered heteroaryl containing 1-3 N atoms.

In some embodiments,

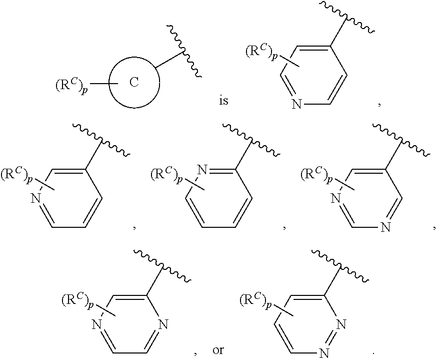

In some embodiments, ring C is a monocyclic 5-membered C₁-C₄heteroaryl containing 1-4 N atoms, 0 or 1 O or S atom.

In some embodiments, ring C is pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments,

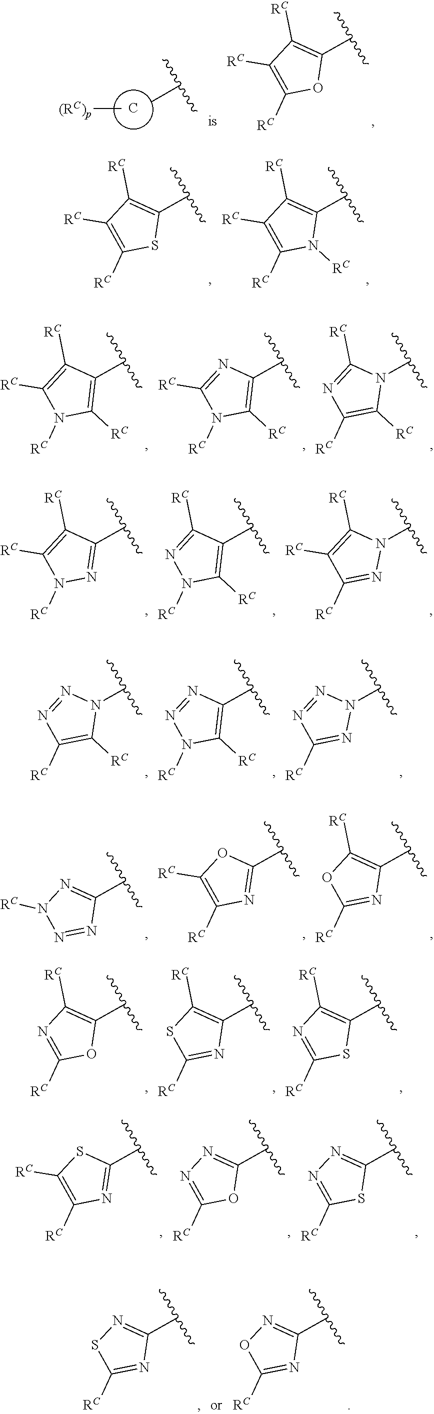

In some embodiments, ring C is monocyclic heterocycle selected from pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, and 1,2,3,6-tetrahydropyridinyl.

In some embodiments, ring C is a monocyclic C₂-C₈heterocycloalkyl containing at least 1 N atom in the ring.

In some embodiments, ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, or azepanyl.

In some embodiments,

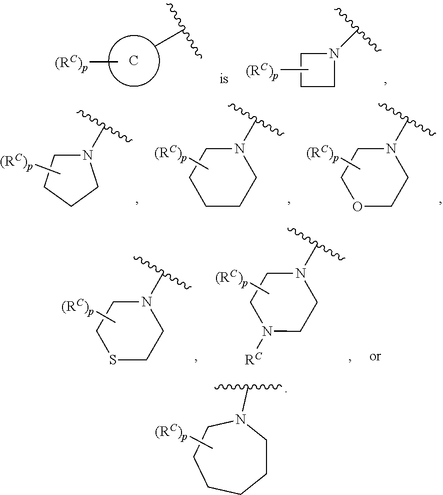

In some embodiments, the compound of Formula (IV) has the structure of Formula (V), or a pharmaceutically acceptable salt or solvate thereof:

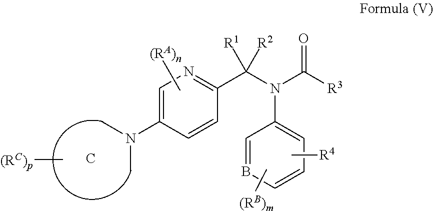

wherein,
ring C is a 5-membered N-containing heteroaryl, or a N-containing C₂-C₈heterocycloalkyl.

In some embodiments, ring C is a 5-membered N-containing heteroaryl containing 1-4 N atoms.

In some embodiments,

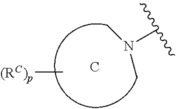

is a monocyclic 5-membered C₁-C₄heteroarylene containing 1-4 N atoms that has the structure

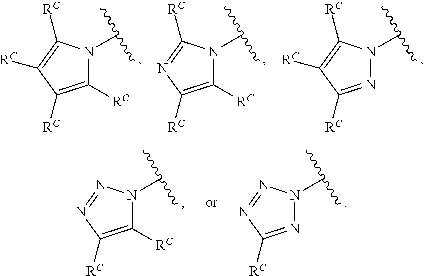

In some embodiments, ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, or azepanyl.

In some embodiments,

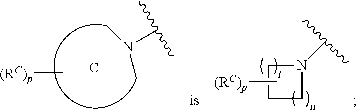

wherein, t is 1, 2, or 3; u is 1, 2, or 3.

In some embodiments,

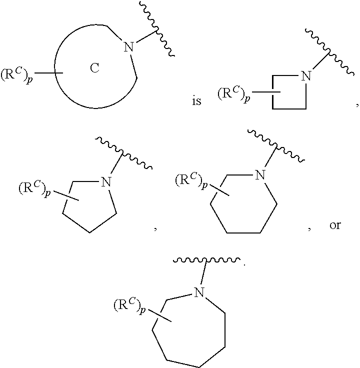

In some embodiments, ring C is a monocyclic C₂-C₈heterocycloalkyl containing 1 N atom in the ring that is selected from a γ-lactam, γ-lactam, δ-lactam or ε-lactam.

In some embodiments, ring C is a bicyclic C₅-C₈heterocycloalkyl that is a fused bicyclic C₅-C₈heterocycloalkyl, bridged bicyclic C₅-C₈heterocycloalkyl, or spiro bicyclic C₅-C₈heterocycloalkyl.

In some embodiments,

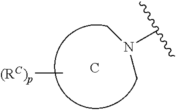

is a bridged bicyclic C₅-C₈heterocycloalkyl that is

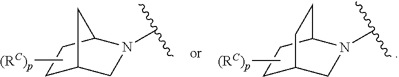

In some embodiments, ring C is a spiro bicyclic C₅-C₈heterocycloalkyl such that

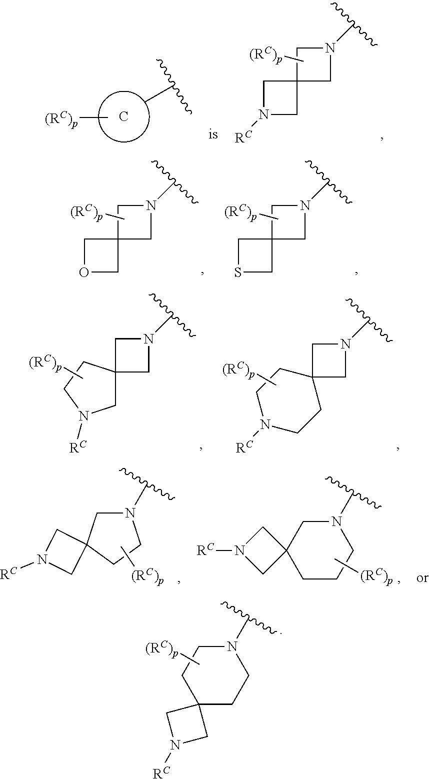

In some embodiments, R³ is selected from substituted or unsubstituted C₁-C₁₀alkyl, substituted or unsubstituted C₃-C₁₀cycloalkyl, or substituted or unsubstituted aryl, wherein if R³ is substituted then R³ is substituted with one or more R¹² groups.

In some embodiments, R³ is selected from methyl, ethyl, propyl, iso-propyl, n-butyl, iso-butyl, tert-butyl, substituted or unsubstituted cyclopropyl, substituted or unsubstituted cyclobutyl, substituted or unsubstituted cyclopentyl, substituted or unsubstituted cyclohexyl, substituted or unsubstituted phenyl,

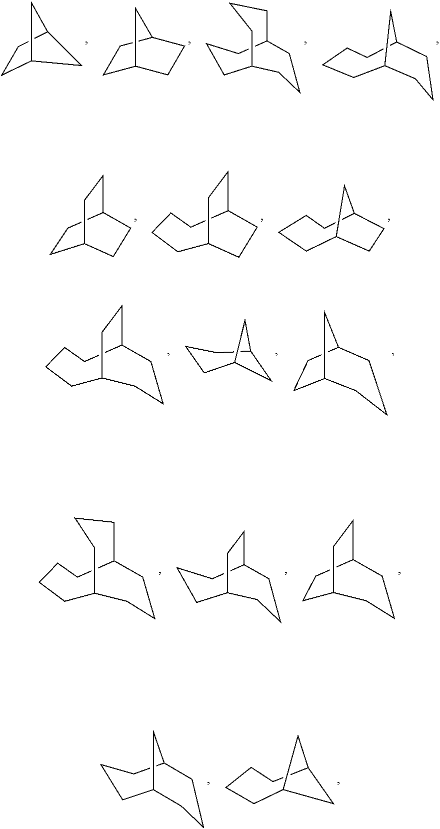

and adamantyl.

In another aspect, described herein is a compound that has the structure of Formula (VI), or a pharmaceutically acceptable salt or solvate thereof:

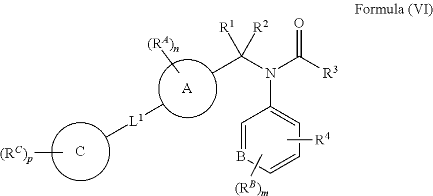

wherein,
R¹ and R² are each independently selected from H, D, F, C₁-C₄alkyl, or C₁-C₄fluoroalkyl;
or R¹ and R² are taken together with die carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹—C(═O)N(R¹⁰)₂, —OC(═O)N(R¹⁰)₂, —NHC(═O)R¹¹, —NHC(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂—, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
   R⁵ and R⁷ are each independently selected from H, D, C₁-C₄alkyl and C₃-C₆cycloalkyl;
   or R⁵ and R⁷ are taken together with the intervening atoms to form a double bond;
   or R⁵ and R⁷ are taken together with the intervening atoms to form an epoxide or an substituted or unsubstituted C₃-C₆cycloalkyl;
   R⁶ and R⁸ are each independently selected from H, D, C₁-C₄alkyl or C₃-C₆cycloalkyl;
   Y is —CH₂OR⁹,

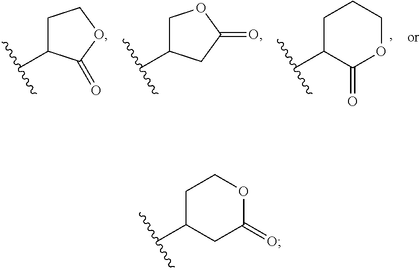 [SMILES: *C1CCOC1=O ; *C1COC(=O)C1 ; *C1CCCOC1=O ; "," ; "or" ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
L¹ is —X¹-L²- or -L²-X¹—;
   X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
   L² is absent or substituted or unsubstituted C₁-C₄alkylene;
ring A is a monocyclic 5-membered heteroarylene;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰), —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
ring C is monocyclic carbocycle, bicyclic carbocycle, monocyclic heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰), —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl, and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

In some embodiments, ring A is a monocyclic 5-membered C₁-C₄heteroarylene containing 1-4 N atoms, 0 or 1 O or S atom.

In some embodiments, ring A is a monocyclic 5-membered C₁-C₄heteroarylene containing 0-4 N atoms, 1 O or S atom.

In some embodiments, ring A is a furanylene, thienylene, pyrrolylene, oxazolylene, thiazolylene, imidazolylene, pyrazolylene, triazolylene, tetrazolyl ene, isoxazolylene, isothiazolylene, oxadiazolylene, or thiadiazolylene.

In some embodiments,

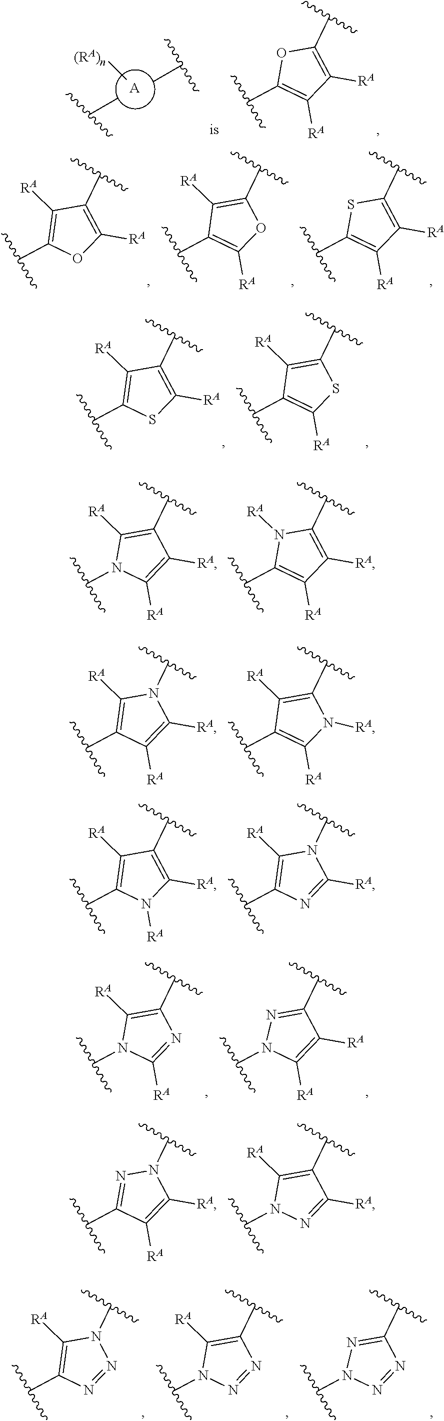

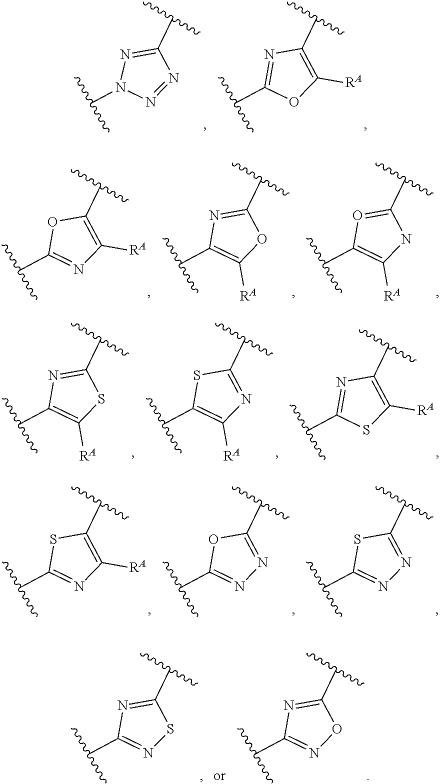

In some embodiments, L¹ is —X¹-L²-, -L²-X¹—; X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, or —NR¹⁰—; L² is absent or —CH₂—.

In some embodiments, L¹ is absent, —O—, —S—, —S—CH₂—, —CH₂—S—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, —NR¹⁰—, —NR¹⁰—CH₂—, or —CH₂—NR¹⁰—.

In some embodiments, the compound of Formula (VI) has the structure of Formula (VII), or a pharmaceutically acceptable salt or solvate thereof:

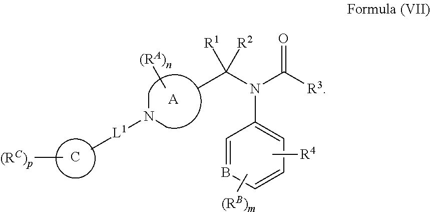

In some embodiments,

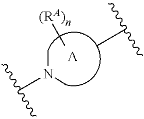

is a monocyclic 5-membered C₁-C₄heteroarylene containing 1-4 N atoms that has the structure

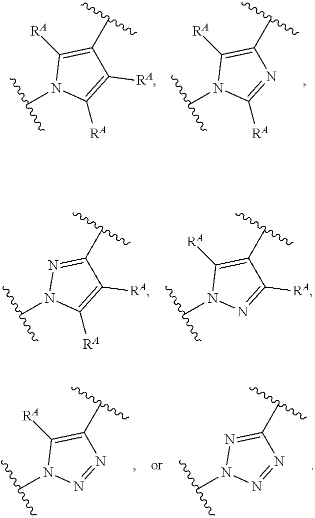

In some embodiments, L¹ is —X¹-L²-, or —V—X¹—; X¹ is absent, —S(═O)—, —S(═O)₂—, —C(═O)—, —OC(═O)—, —NR¹⁰C(═O)—, or —NR¹⁰S(═O)₂—; L² is absent or substituted or unsubstituted C₁-C₄alkylene.

In yet another aspect, described herein is a compound that has the structure of Formula (VIII), or a pharmaceutically acceptable salt or solvate thereof:

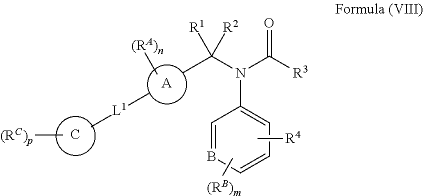

wherein,
R¹ and R² are each independently selected from H, D, F, C₁-C₄alkyl, and C₁-C₄fluoroalkyl;
or R¹ and R² are taken together with die carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O); R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NHC(═O)R¹¹, —NHC(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂—, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
   L³ is —C(R⁵)(R⁶)—, —C(R⁵)(R⁶)—C(R⁷)(R⁸)—, —O—C(R⁷)(R⁸)—, or —C(R⁵)(R⁶)—O—;
   R⁵ and R⁷ are each independently selected from H, D, C₁-C₄alkyl, and C₃-C₆cycloalkyl;
   or R⁵ and R⁷ are taken together with the intervening atoms to form a double bond;
   or R⁵ and R⁷ are taken together with the intervening atoms to form an epoxide or an substituted or unsubstituted C₃-C₆cycloalkyl;
   R⁶ and R⁸ are each independently selected from H, D, C₁-C₄alkyl, or C₃-C₆cycloalkyl;
   Y is —CH₂OR⁹,

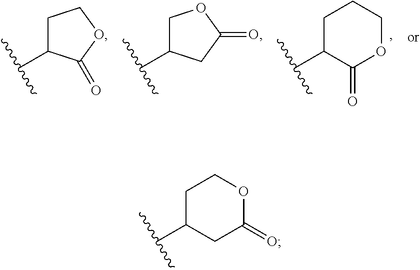 [SMILES: *C1CCOC1=O ; *C1COC(=O)C1 ; *C1CCCOC1=O ; "," ; "or" ; *C1CCOC(=O)C1]

R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
L¹ is —X¹-L²-, -L²-X¹—;
   X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
   L² is absent or substituted or unsubstituted C₁-C₄alkylene;
ring A is a C₃-C₁₀cycloalkyl;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl,
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

In some embodiments, ring A is C₃-C₁₀cycloalkyl that is selected from cyclopropyl, cyclobutyl, cyclopentyl, cyclohexyl, cycloheptyl, and cyclooctyl.

In some embodiments, ring A is

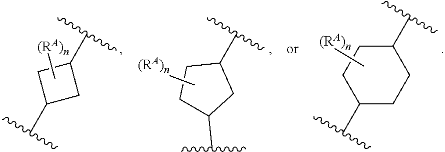

In some embodiments, L¹ is —X¹-L²-, -L²-X¹—; X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, or —NR¹⁰—; L² is absent or —CH₂—.

In some embodiments, L¹ is absent, —O—, —S—, —S—CH₂—, —CH₂—S—, —CH₂—, —CH═CH—, —C≡C—, —C(═O)—, —C(═O)O—, —OC(═O)—, —C(═O)NR¹⁰—, —NR¹⁰C(═O)—, —NR¹⁰C(═O)—, —NR¹⁰—, —NR¹⁰—CH₂—, or —CH₂—NR¹⁰—.

In some embodiments, R⁴ is

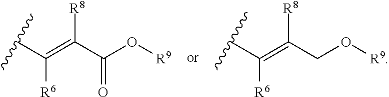

In some embodiments, R⁴

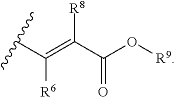

In some embodiments, R⁴ is

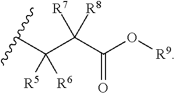

In some embodiments, R⁴ is

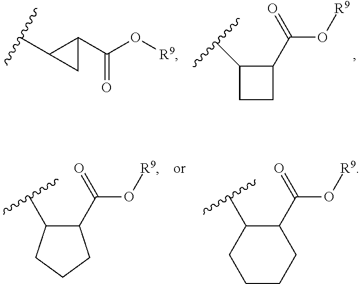

In some embodiments, R⁴ is -L³-Y; L³ is —CH₂—; Y is

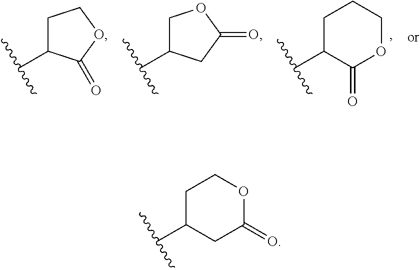 [SMILES: *C1CCOC1=O ; *C1COC(=O)C1 ; *C1CCCOC1=O ; "," ; "or" ; *C1CCOC(=O)C1]

In some embodiments, R⁹ is selected from H, substituted or unsubstituted C₁-C₆alkyl, substituted or unsubstituted C₁-C₆heteroalkyl, substituted or unsubstituted C₃-C₆cycloalkyl, substituted or unsubstituted phenyl, and substituted or unsubstituted heterocycle. In some embodiments, R⁹ is selected from substituted or unsubstituted C₁-C₆alkyl, or substituted or unsubstituted C₁-C₆heteroalkyl. In some embodiments, R⁹ is substituted or unsubstituted C₁-C₆alkyl.

In some embodiments, R⁹ is substituted or unsubstituted C₁-C₆heteroalkyl. In some embodiments, R⁹ is selected from substituted or unsubstituted C₃-C₆cycloalkyl, substituted or unsubstituted phenyl, and substituted or unsubstituted heterocycle. In some embodiments, R⁹ is substituted or substituted or unsubstituted heterocycle. In some embodiments, R⁹ is selected from H, C₁-C₆alkyl, C₂-C₆alkenyl, and C₃-C₆cycloalkyl.

In some embodiments, R⁹ is methyl, ethyl, propyl, iso-propyl, n-butyl, iso-butyl, tert-butyl, n-pentyl, tert-pentyl, neopentyl, iso-pentyl, sec-pentyl, 3-pentyl, n-hexyl, iso-hexyl, 3-methylpentyl, 2,3-dimethylbutyl, or neohexyl.

In some embodiments, ring C is monocyclic carbocycle or bicyclic carbocycle.

In some embodiments, ring C is monocyclic carbocycle selected from cyclopropyl, cyclobutyl, cyclopentyl, cyclohexyl, and phenyl.

In some embodiments, ring C is phenyl.

In some embodiments, ring C is bicyclic carbocycle selected from indanyl, indenyl, and naphthyl.

In some embodiments, ring C is monocyclic heterocycle or bicyclic heterocycle.

In some embodiments, ring C is monocyclic heterocycle or bicyclic heterocycle selected from pyridinyl, pyrazolyl, pyrrolyl, imidazolyl, oxazolyl, isoxazolyl, thiazolyl, isothiazolyl, triazolyl, pyrimidinyl, pyrazinyl, triazinyl, benzimidazolyl, indolyl, quinolinyl, indazolyl, purinyl, quinoxalinyl, and acridinyl.

In some embodiments,

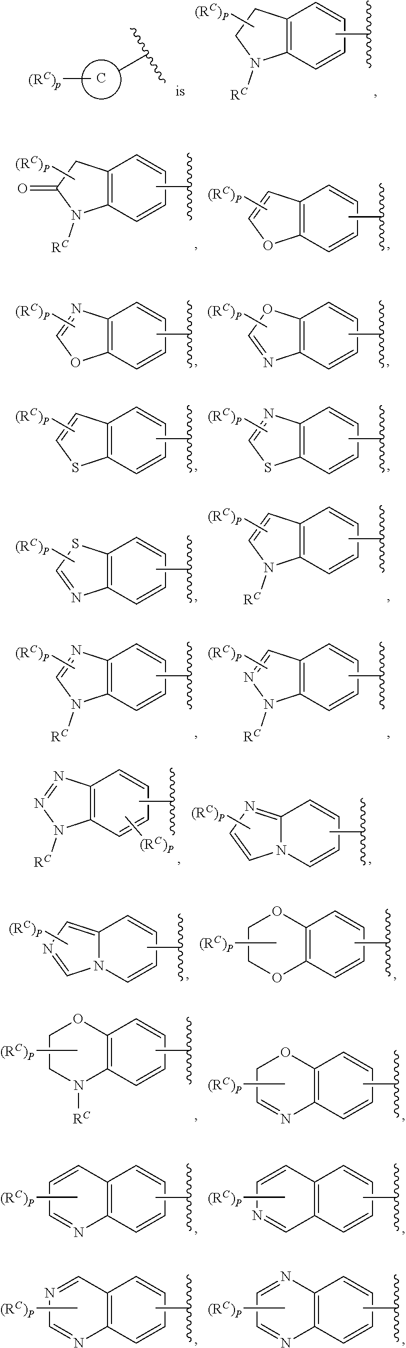

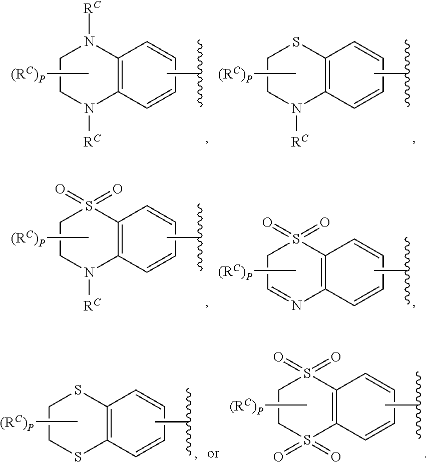

In some embodiments, ring C is monocyclic heteroaryl selected from furanyl, thienyl, pyridinyl, pyrazolyl, pyrrolyl, imidazolyl, oxazolyl, isoxazolyl, thiazolyl, isothiazolyl, triazolyl, pyrimidinyl, pyrazinyl, and triazinyl.

In some embodiments, ring C is a monocyclic 6-membered heteroaryl containing 1-3 N atoms.

In some embodiments,

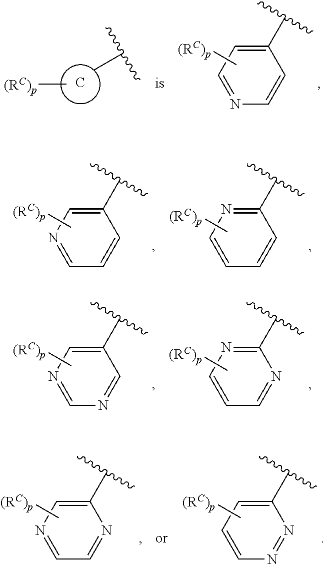

In some embodiments, ring C is a monocyclic 5-membered C₁-C₄heteroaryl.

In some embodiments, ring C is pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments,

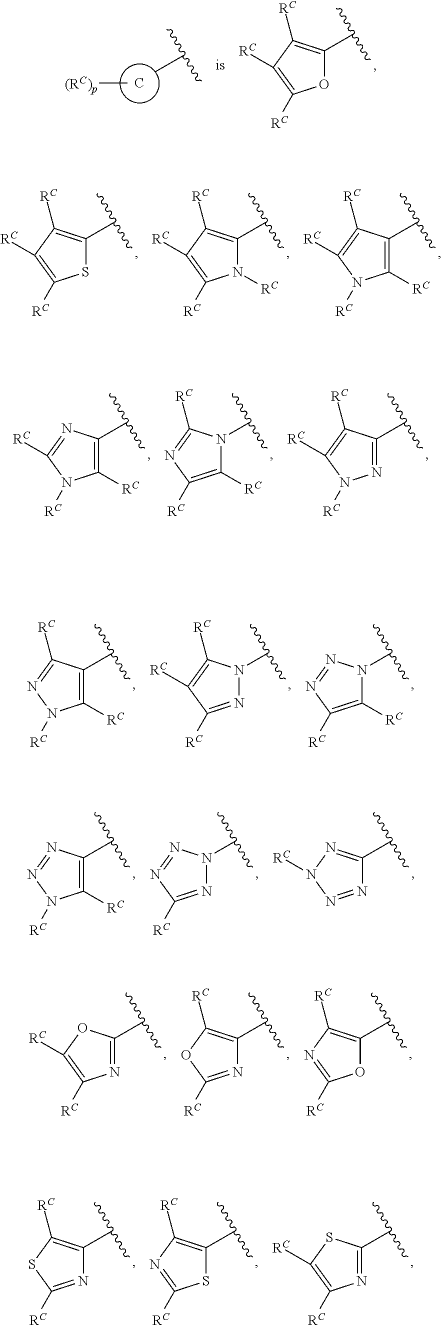

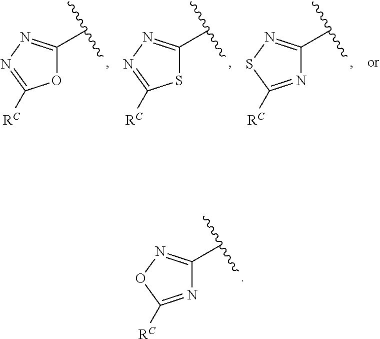

In some embodiments, ring C is monocyclic heterocycle selected from pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, and 1,2,3,6-tetrahydropyridinyl.

In some embodiments, ring C is a monocyclic C₂-C₈heterocycloalkyl containing at least 1 N atom in the ring.

In some embodiments, ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, and azepanyl.

In some embodiments,

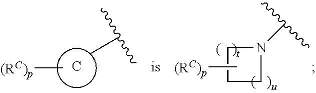

wherein, t is 1, 2, or 3; u is 1, 2, or 3.

In some embodiments,

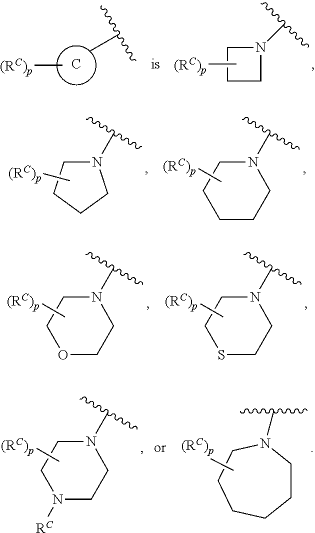

In some embodiments, ring C is a C₂-C₈heterocycloalkyl containing 1 N atom in the ring that is selected from a β-lactam, γ-lactam, δ-lactam or ε-lactam.

In some embodiments, ring C is a bicyclic C₅-C₈heterocycloalkyl that is a fused bicyclic C₅-C₈heterocycloalkyl, bridged bicyclic C₅-C₈heterocycloalkyl, or spiro bicyclic C₅-C₈heterocycloalkyl.

In some embodiments,

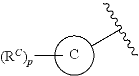

is a bridged bicyclic C₅-C₈heterocycloalkyl that is

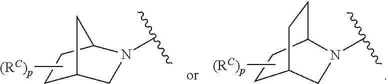

In some embodiments,

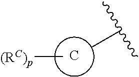

is spiro bicyclic C₅-C₈heterocycloalkyl that is

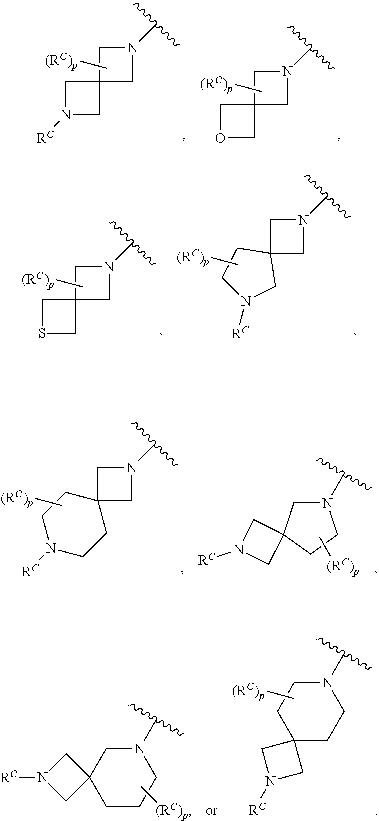

In some embodiments, R³ is selected from substituted or unsubstituted C₁-C₁₀alkyl, substituted or unsubstituted C₃-C₁₀cycloalkyl, or substituted or unsubstituted aryl, wherein if R³ is substituted then R³ is substituted with one or more R¹² groups.

In some embodiments, R³ is selected from methyl, ethyl, propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, neohexyl, substituted or unsubstituted cyclopropyl, substituted or unsubstituted cyclobutyl, substituted or unsubstituted cyclopentyl, substituted or unsubstituted cyclohexyl, substituted or unsubstituted phenyl,

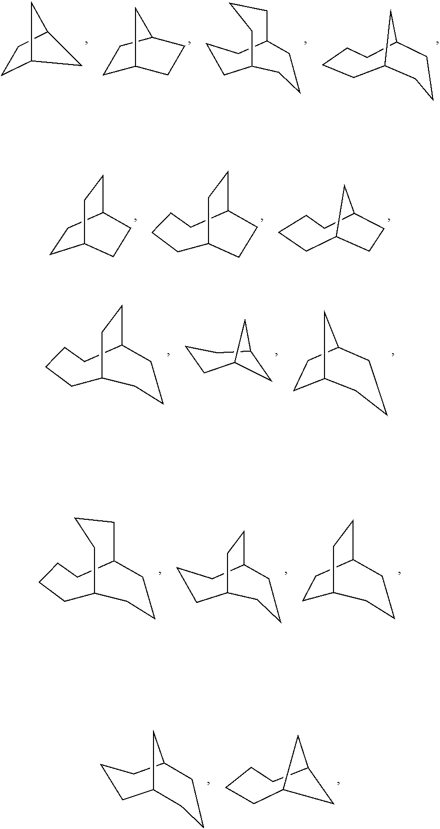

and adamantyl.

In some embodiments, R³ is selected from methyl, ethyl, propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, neohexyl, substituted or unsubstituted cyclopropyl, substituted or unsubstituted cyclobutyl, substituted or unsubstituted cyclopentyl, substituted or unsubstituted cyclohexyl, and substituted or unsubstituted phenyl.

In some embodiments, R³ is selected from methyl, ethyl, propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, and neohexyl.

In some embodiments, R³ is selected from substituted or unsubstituted cyclopropyl, substituted or unsubstituted cyclobutyl, substituted or unsubstituted cyclopentyl, substituted or unsubstituted cyclohexyl, and substituted or unsubstituted phenyl.

In some embodiments, R³ is selected from methyl, ethyl, propyl, iso-propyl, n-butyl, iso-butyl, tert-butyl, substituted or unsubstituted cyclopropyl, substituted or unsubstituted cyclobutyl, substituted or unsubstituted cyclopentyl, and substituted or unsubstituted cyclohexyl, substituted or unsubstituted phenyl,

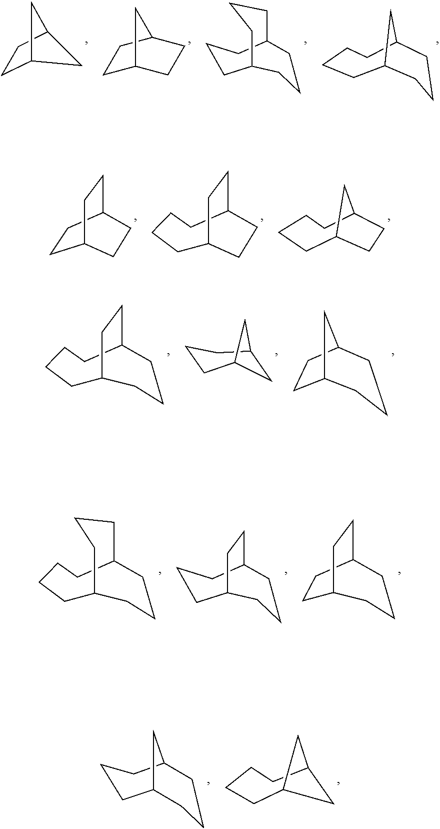

and adamantyl.

In some embodiments, R³ is selected from substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups.

In some embodiments, R³ is selected from substituted or unsubstituted C₃-C₁₀cycloalkyl, substituted or unsubstituted monocyclic C₂-C₁₀heterocycloalkyl, substituted or unsubstituted phenyl, or substituted or unsubstituted monocyclic heteroaryl, wherein if R³ is substituted then R³ is substituted with one or more R¹² groups. In some embodiments, R³ is selected from substituted or unsubstituted C₂-C₁₀heterocycloalkyl, substituted or unsubstituted monocyclic heteroaryl, wherein if R³ is substituted then R³ is substituted with one or more R¹² groups.

In yet another aspect, described herein is a compound that has the structure of Formula (IX), or a pharmaceutically acceptable salt or solvate thereof:

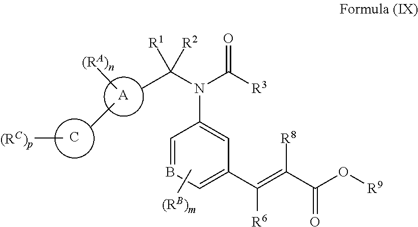

wherein,
R¹ and R² are each independently selected from H, D, F, C₁-C₄alkyl, or C₁-C₄fluoroalkyl;
R³ is selected from substituted or unsubstituted C₁-C₁₀alkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
   each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NR¹⁰C(═O)R¹¹, —NR¹⁰C(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
   L⁴ is absent, —O—, —S—, —S(═O)—, —S(═O)₂₋, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
   L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
   R¹³ is H, halogen, —N(R¹⁰)₂, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁶ and R⁸ are each independently selected from H, D, C₁-C₄alkyl, and C₃-C₆cycloalkyl;
R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
ring A is a monocyclic C₃-C₆cycloalkyl;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂(R¹⁰), —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
ring C is monocyclic carbocycle or monocyclic heterocycle;
each R^{C} is independently selected from H, D, halogen, —CN, —OH, —OR¹⁰, —SR¹⁰, —S(═O)R¹¹, —NO₂, —N(R¹⁰), —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl, and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

In some embodiments, R¹ and R² are each independently selected from H and D; R³ is selected from substituted or unsubstituted C₁-C₁₀alkyl, substituted or unsubstituted C₃-C₆cycloalkyl, and substituted or unsubstituted phenyl, wherein if R³ is substituted then R³ is substituted with one or more R¹² groups; R⁶ and R⁸ are each independently selected from H, D, —CH₃; R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle; ring A is cyclohexyl; B is CR^{B} or N; ring C is phenyl or monocyclic heterocycle.

Any combination of the groups described above for the various variables is contemplated herein. Throughout the specification, groups and substituents thereof are chosen by one skilled in the field to provide stable moieties and compounds.

In some embodiments, compounds described herein include, but are not limited to, those described in Table 1 and Table 2.

TABLE 1

| Compound | | |
|---|---|---|
| No | Structure | Chemical Name |
| 1 | 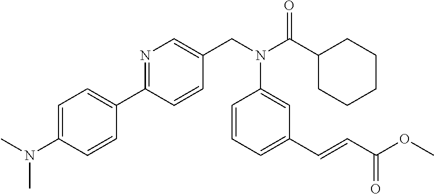 | (E)-Methyl 3-(3-(N-((6-(4- (dimethylamino)phenyl)pyridin-3- yl)methyl)cyclohexanecarboxamido)phenyl) acrylate |
| 2 | 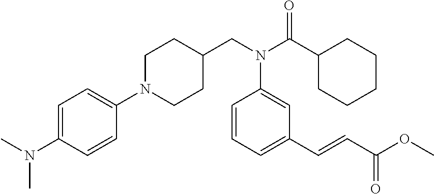 | (E)-Methyl 3-(3-(N-((1-(4- (dimethylamino)phenyl)piperidin-4- yl)methyl)cyclohexanecarboxamido)phenyl) acrylate |
| 3 | 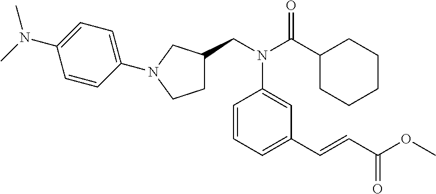 | (R,E)-Methyl 3-(3-(N-((1-(4- (dimethylamino)phenyl)pyrrolidin-3- yl)methyl)cyclohexanecarboxamido)phenyl) acrylate |
| 4 | 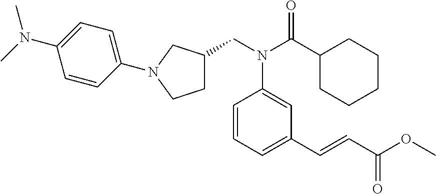 | (S,E)-Methyl 3-(3-(N-((1-(4- (dimethylamino)phenyl)pyrrolidin-3- yl)methyl)cyclohexanecarboxamido)phenyl) acrylate |
| 4.1 | 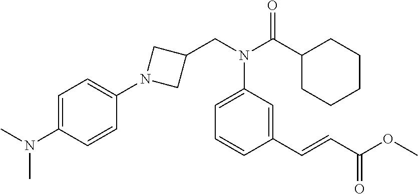 | (E)-Methyl 3-(3-(N-((1-(4- (dimethylamino)phenyl)azetidin-3- yl)methyl)cyclohexanecarboxamido)phenyl) acrylate |
| 5 | 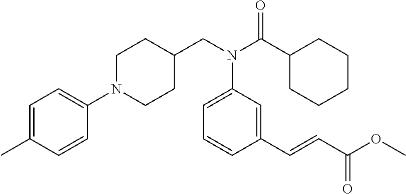 | (E)-Methyl 3-(3-(N-((1-(p-tolyl)piperidin-4- yl)methyl)cyclohexanecarboxamido)phenyl) acrylate |
| 6 | 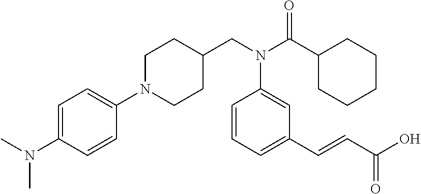 [SMILES: CN(C)c1ccc(N2CCC(CN(C(=O)C3CCCCC3)c3cccc(/C=C/C(=O)O)c3)CC2)cc1] | (E)-3-(3-(N-((1-(4- (Dimethylamino)phenyl)piperidin-4- yl)methyl)cyclohexanecarboxamido)phenyl) acrylic acid |
| 6.1 | 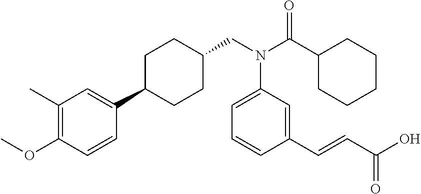 [SMILES: COc1ccc([C@H]2CC[C@H](CN(C(=O)C3CCCCC3)c3cccc(/C=C/C(=O)O)c3)CC2)cc1C] | trans-(E)-3-(3-(N-((4-(4-Methoxy-3- methylphenyl)cyclohexyl)methyl) cyclohexanecarboxamido)phenyl)acrylic acid |
| 7 | 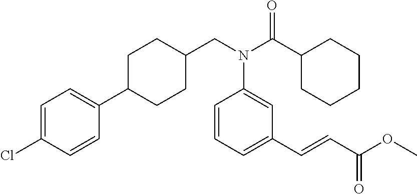 [SMILES: COC(=O)/C=C/c1cccc(N(CC2CCC(c3ccc(Cl)cc3)CC2)C(=O)C2CCCCC2)c1] | (E)-Methyl 3-(3-(N-((4-(4- chlorophenyl)cyclohexyl)methyl) cyclohexanecarboxamido)phenyl)acrylate |
| 8 | 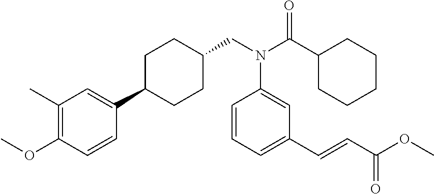 | trans-(E)-Methyl 3-(3-(N-((4-(4-methoxy-3- methylphenyl)cyclohexyl)methyl) cyclohexanecarboxamido)phenyl)acrylate |
| 9 | 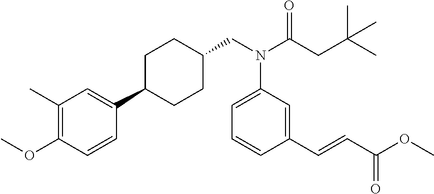 | trans-(E)-Methyl 3-(3-(N-((4-(4-methoxy-3- methylphenyl)cyclohexyl)methyl)-3,3- dimethylbutanamido)phenyl)acrylate |
| 10 | 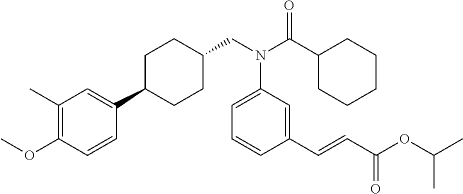 [SMILES: COc1ccc([C@H]2CC[C@H](CN(C(=O)C3CCCCC3)c3cccc(/C=C/C(=O)OC(C)C)c3)CC2)cc1C] | trans-(E)-Isopropyl 3-(3-(N-((4-(4-methoxy-3- methylphenyl)cyclohexyl)methyl) cyclohexanecarboxamido)phenyl)acrylate |
| 11 | 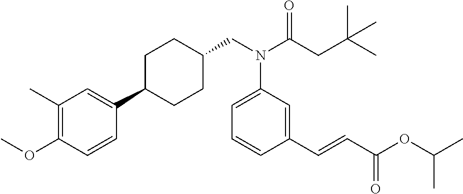 [SMILES: COc1ccc([C@H]2CC[C@H](CN(C(=O)CC(C)(C)C)c3cccc(/C=C/C(=O)OC(C)C)c3)CC2)cc1C] | trans-(E)-Isopropyl 3-(3-(N-((4-(4-methoxy-3- methylphenyl)cyclohexyl)methyl)-3,3- dimethylbutanamido)phenyl)acrylate |
| 12 | 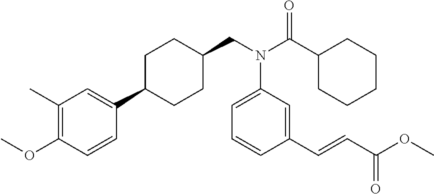 | cis-(E)-Methyl 3-(3-(N-((4-(4-methoxy-3- methylphenyl)cyclohexyl)methyl) cyclohexanecarboxamido)phenyl)acrylate |
| 13 | 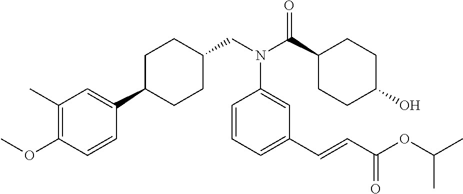 [SMILES: COc1ccc([C@H]2CC[C@H](CN(c3cccc(/C=C/C(=O)OC(C)C)c3)C(=O)[C@H]3CC[C@H](O)CC3)CC2)cc1C] | trans-(E)-Isopropyl 3-(3-(trans-4-hydroxy-N- ((4-(4-methoxy-3- methylphenyl)cyclohexyl)methyl) cyclohexanecarboxamido)phenyl)acrylate |
| 14 | 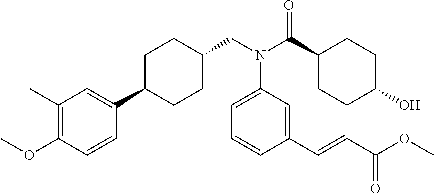 | trans-(E)-Methyl 3-(3-(trans-4-hydroxy-N-((4- (4-methoxy-3- methylphenyl)cyclohexyl)methyl) cyclohexanecarboxamido)phenyl)acrylate |

TABLE 2

| Structure | Name |
|---|---|
| 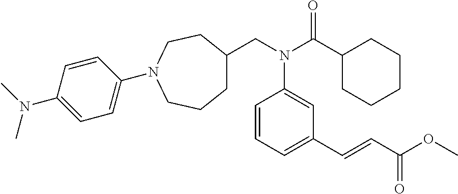 | (E)-Methyl 3-(3-(N-((1-(4- (dimethylamino)phenyl)azepan-4- yl)methyl)cyclohexanecarboxamido)phenyl)acrylate |
| 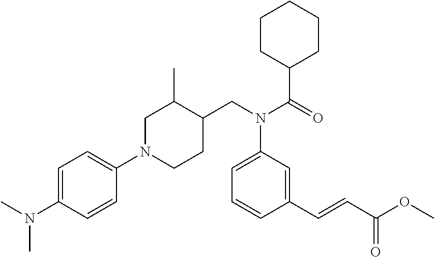 | (E)-Methyl 3-(3-(N-((1-(4-(dimethylamino)phenyl)- 3-methylpiperidin-4- yl)methyl)cyclohexanecarboxamido)phenyl)acrylate |
| 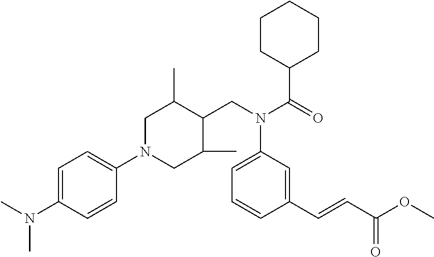 | (E)-Methyl 3-(3-(N-((1-(4-(dimethylamino)phenyl)- 3,5-dimethylpiperidin-4- yl)methyl)cyclohexanecarboxamido)phenyl)acrylate |
| 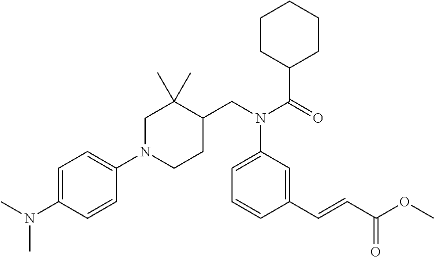 | (E)-Methyl 3-(3-(N-((1-(4-(dimethylamino)phenyl)- 3,3-dimethylpiperidin-4- yl)methyl)cyclohexanecarboxamido)phenyl)acrylate |
| 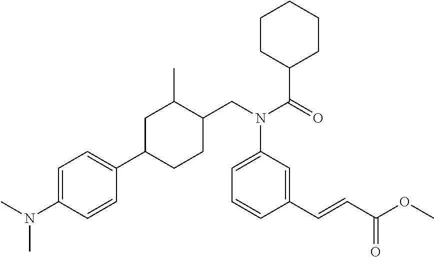 | (E)-Methyl 3-(3-(N-((4-(4-(dimethylamino)phenyl)- 2- methylcyclohexyl)methyl)cyclohexanecarboxamido) phenyl)acrylate |
| 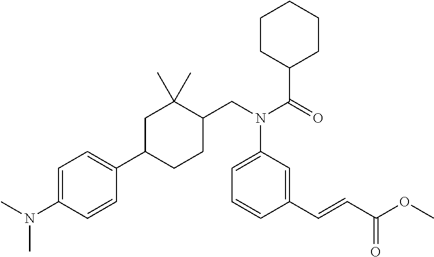 | (E)-Methyl 3-(3-(N-((4-(4-(dimethylamino)phenyl)- 2,2- dimethylcyclohexyl)methyl)cyclohexanecarboxamido) phenyl)acrylate |
| 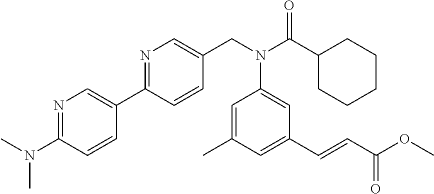 | (E)-Methyl 3-(3-(N-((6′-(dimethylamino)-[2,3′- bipyridin]-5-yl)methyl)cyclohexanecarboxamido)-5- methylphenyl)acrylate |
| 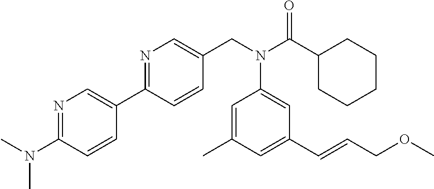 | (E)-N-((6′-(Dimethylamino)-[2,3′-bipyridin]-5- yl)methyl)-N-(3-(3-methoxyprop-1-en-1-yl)-5- methylphenyl)cyclohexanecarboxamide |
| 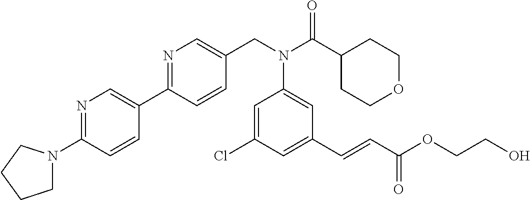 [SMILES: O=C(/C=C/c1cc(Cl)cc(N(Cc2ccc(-c3ccc(N4CCCC4)nc3)nc2)C(=O)C2CCOCC2)c1)OCCO] | (E)-2-Hydroxyethyl 3-(3-chloro-5-(N-((6′-(pyrrolidin- 1-yl)-[2,3′-bipyridin]-5-yl)methyl)tetrahydro-2H- pyran-4-carboxamido)phenyl)acrylate |
| 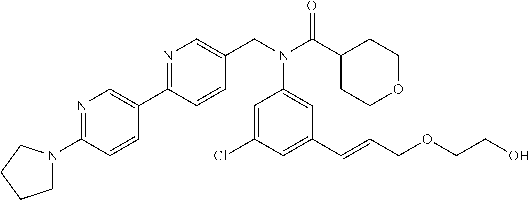 [SMILES: O=C(C1CCOCC1)N(Cc1ccc(-c2ccc(N3CCCC3)nc2)nc1)c1cc(Cl)cc(/C=C/COCCO)c1] | (E)-N-(3-Chloro-5-(3-(2-hydroxyethoxy)prop-1-en-1- yl)phenyl)-N-((6′-(pyrrolidin-1-yl)-[2,3′-bipyridin]-5- yl)methyl)tetrahydro-2H-pyran-4-carboxamide |
| 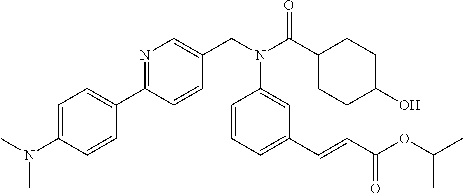 [SMILES: CC(C)OC(=O)/C=C/c1cccc(N(Cc2ccc(-c3ccc(N(C)C)cc3)nc2)C(=O)C2CCC(O)CC2)c1] | (E)-Isopropyl 3-(3-(N-((6-(4- (dimethylamino)phenyl)pyridin-3-yl)methyl)-4- hydroxycyclohexanecarboxamido)phenyl)acrylate |
| 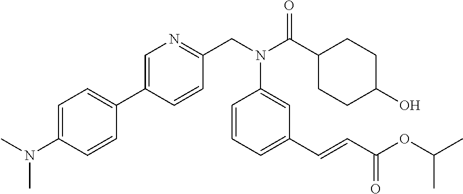 [SMILES: CC(C)OC(=O)/C=C/c1cccc(N(Cc2ccc(-c3ccc(N(C)C)cc3)cn2)C(=O)C2CCC(O)CC2)c1] | (E)-Isopropyl 3-(3-(N-((5-(4- (dimethylamino)phenyl)pyridin-2-yl)methyl)-4- hydroxycyclohexanecarboxamido)phenyl)acrylate |
| 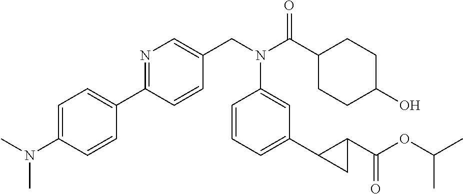 [SMILES: CC(C)OC(=O)C1CC1c1cccc(N(Cc2ccc(-c3ccc(N(C)C)cc3)nc2)C(=O)C2CCC(O)CC2)c1] | Isopropyl 2-(3-(N-((6-(4- (dimethylamino)phenyl)pyridin-3-yl)methyl)-4- hydroxycyclohexanecarboxamido)phenyl) cyclopropanecarboxylate |
| 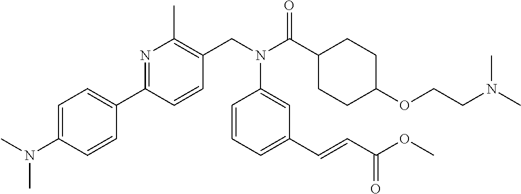 | (E)-Methyl 3-(3-(4-(2-(dimethylamino)ethoxy)-N- ((6-(4-(dimethylamino)phenyl)-2-methylpyridin-3- yl)methyl)cyclohexanecarboxamido)phenyl)acrylate |
| 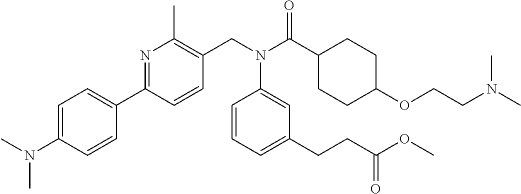 | Methyl 3-(3-(4-(2-(dimethylamino)ethoxy)-N-((6-(4- (dimethylamino)phenyl)-2-methylpyridin-3- yl)methyl)cyclohexanecarboxamido)phenyl)propanoate |
| 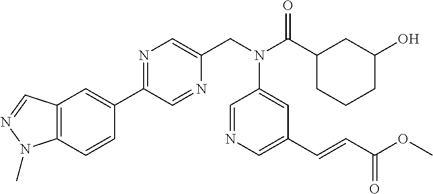 [SMILES: COC(=O)/C=C/c1cncc(N(Cc2cnc(-c3ccc4c(cnn4C)c3)cn2)C(=O)C2CCCC(O)C2)c1] | (E)-Methyl 3-(5-(3-hydroxy-N-((5-(1-methyl-1H- indazol-5-yl)pyrazin-2- yl)methyl)cyclohexanecarboxamido)pyridin-3- yl)acrylate |
| 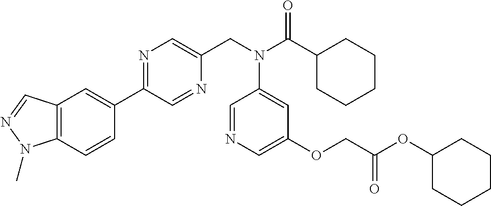 [SMILES: Cn1ncc2cc(-c3cnc(CN(C(=O)C4CCCCC4)c4cncc(OCC(=O)OC5CCCCC5)c4)cn3)ccc21] | Cyclohexyl 2-((5-(N-((5-(1-methyl-1H-indazol-5- yl)pyrazin-2- yl)methyl)cyclohexanecarboxamido)pyridin-3- yl)oxy)acetate |
| 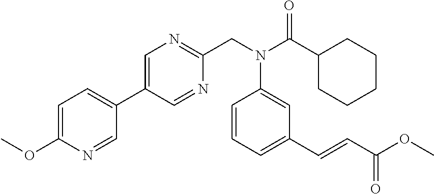 | (E)-Methyl 3-(3-(N-((5-(6-methoxypyridin-3- yl)pyrimidin-2- yl)methyl)cyclohexanecarboxamido)phenyl)acrylate |
| 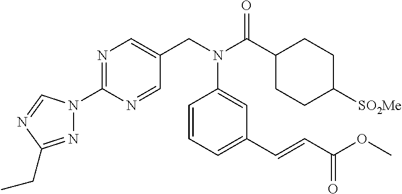 [SMILES: CCc1ncn(-c2ncc(CN(C(=O)C3CCC(S(C)(=O)=O)CC3)c3cccc(/C=C/C(=O)OC)c3)cn2)n1] | (E)-Methyl 3-(3-(N-((2-(3-ethyl-1H-1,2,4-triazol-1- yl)pyrimidin-5-yl)methyl)-4-(methylsulfonyl) cyclohexanecarboxamido)phenyl)acrylate |
| 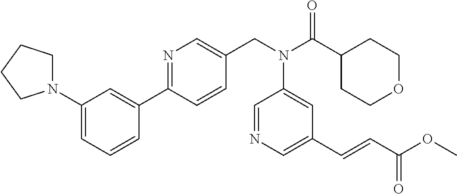 [SMILES: COC(=O)/C=C/c1cncc(N(Cc2ccc(-c3cccc(N4CCCC4)c3)nc2)C(=O)C2CCOCC2)c1] | (E)-Methyl 3-(5-(N-((6-(3-(pyrrolidin-1- yl)phenyl)pyridin-3-yl)methyl)tetrahydro-2H-pyran- 4-carboxamido)pyridin-3-yl)acrylate |
| 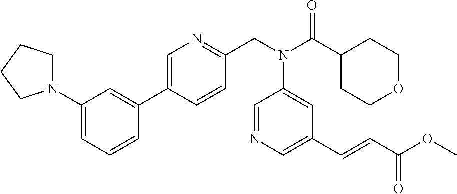 [SMILES: COC(=O)/C=C/c1cncc(N(Cc2ccc(-c3cccc(N4CCCC4)c3)cn2)C(=O)C2CCOCC2)c1] | (E)-Methyl 3-(5-(N-((5-(3-(pyrrolidin-1- yl)phenyl)pyridin-2-yl)methyl)tetrahydro-2H-pyran- 4-carboxamido)pyridin-3-yl)acrylate |
| 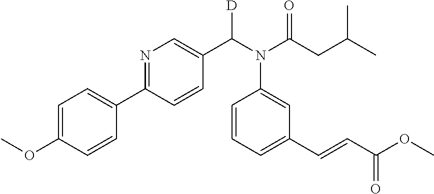 | (E)-Methyl 3-(3-(N-(deutero(6-(4- methoxyphenyl)pyridin-3-yl)methyl)-3- methylbutanamido)phenyl)acrylate |
| 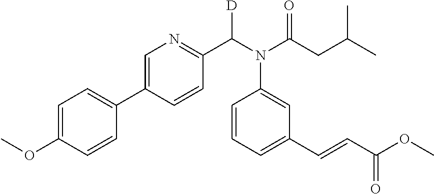 | (E)-Methyl 3-(3-(N-(deutero(5-(4- methoxyphenyl)pyridin-2-yl)methyl)-3- methylbutanamido)phenyl)acrylate |
| 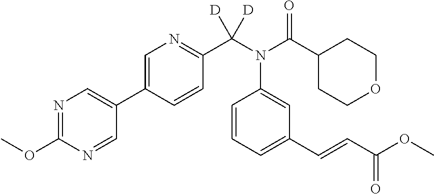 | (E)-methyl 3-(3-(N-(dideutero(5-(2- methoxypyrimidin-5-yl)pyridin-2- yl)methyl)tetrahydro-2H-pyran-4- carboxamido)phenyl)acrylate |
| 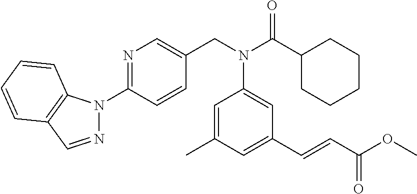 [SMILES: COC(=O)/C=C/c1cc(C)cc(N(Cc2ccc(-n3ncc4ccccc43)nc2)C(=O)C2CCCCC2)c1] | (E)-Methyl 3-(3-(N-((6-(1H-indazol-1-yl)pyridin-3- yl)methyl)cyclohexanecarboxamido)-5- methylphenyl)acrylate |
| 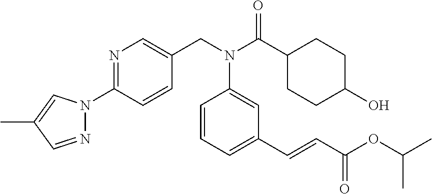 [SMILES: Cc1cnn(-c2ccc(CN(C(=O)C3CCC(O)CC3)c3cccc(/C=C/C(=O)OC(C)C)c3)cn2)c1] | (E)-Isopropyl 3-(3-(4-hydroxy-N-((6-(4-methyl-1H- pyrazol-1-yl)pyridin-3- yl)methyl)cyclohexanecarboxamido)phenyl)acrylate |
| 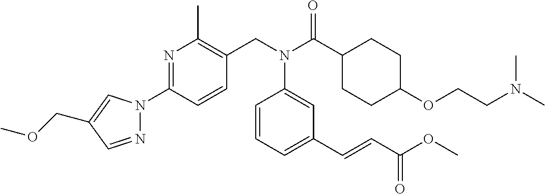 | (E)-Methyl 3-(3-(4-(2-(dimethylamino)ethoxy)-N- ((6-(4-(methoxymethyl)-1H-pyrazol-1-yl)-2- methylpyridin-3- yl)methyl)cyclohexanecarboxamido)phenyl)acrylate |
| 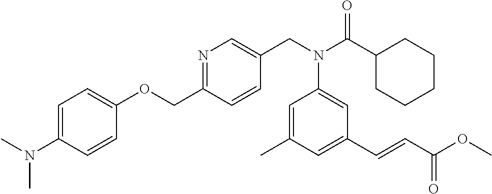 | (E)-Methyl 3-(3-(N-((6-((4- (dimethylamino)phenoxy)methyl)pyridin-3- yl)methyl)cyclohexanecarboxamido)-5- methylphenyl)acrylate |
| 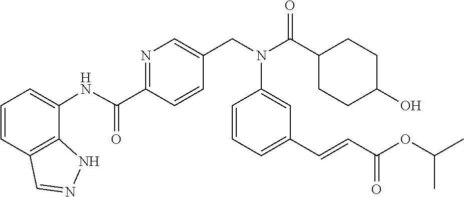 [SMILES: CC(C)OC(=O)/C=C/c1cccc(N(Cc2ccc(C(=O)Nc3cccc4cn[nH]c34)nc2)C(=O)C2CCC(O)CC2)c1] | (E)-Isopropyl 3-(3-(N-((6-((1H-indazol-7- yl)carbamoyl)pyridin-3-yl)methyl)-4- hydroxycyclohexanecarboxamido)phenyl)acrylate |
| 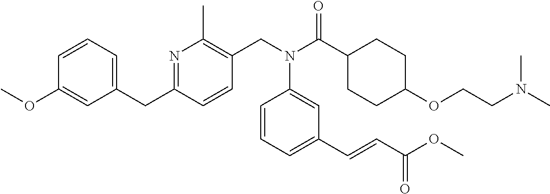 | (E)-Methyl 3-(3-(4-(2-(dimethylamino)ethoxy)-N- ((6-(3-methoxybenzyl)-2-methylpyridin-3- yl)methyl)cyclohexanecarboxamido)phenyl)acrylate |
| 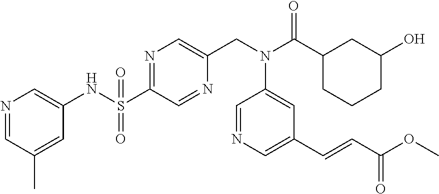 [SMILES: COC(=O)/C=C/c1cncc(N(Cc2cnc(S(=O)(=O)Nc3cncc(C)c3)cn2)C(=O)C2CCCC(O)C2)c1] | (E)-Methyl 3-(5-(3-hydroxy-N-((5-(N-(5- methylpyridin-3-yl)sulfamoyl)pyrazin-2- yl)methyl)cyclohexanecarboxamido)pyridin-3- yl)acrylate |
| 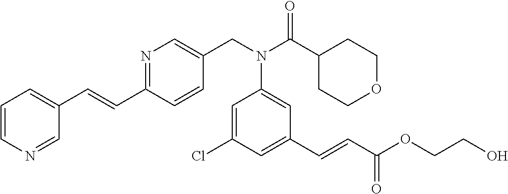 [SMILES: O=C(/C=C/c1cc(Cl)cc(N(Cc2ccc(/C=C/c3cccnc3)nc2)C(=O)C2CCOCC2)c1)OCCO] | (E)-2-Hydroxyethyl 3-(3-chloro-5-(N-((6-((E)-2- (pyridin-3-yl)vinyl)pyridin-3-yl)methyl)tetrahydro- 2H-pyran-4-carboxamido)phenyl)acrylate |
| 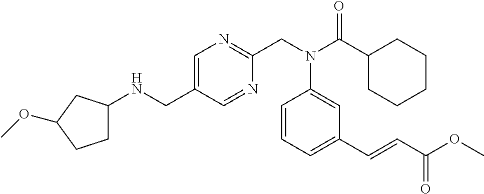 | (E)-Methyl 3-(3-(N-((5-(((3- methoxycyclopentyl)amino)methyl)pyrimidin-2- yl)methyl)cyclohexanecarboxamido)phenyl)acrylate |
| 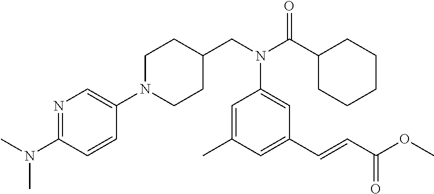 | (E)-Methyl 3-(3-(N-((1-(6-(dimethylamino)pyridin-3- yl)piperidin-4-yl)methyl)cyclohexanecarboxamido)- 5-methylphenyl)acrylate |
| 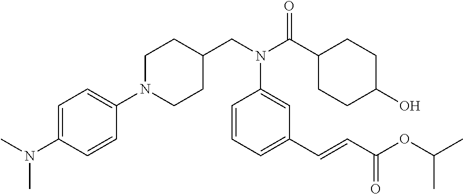 [SMILES: CC(C)OC(=O)/C=C/c1cccc(N(CC2CCN(c3ccc(N(C)C)cc3)CC2)C(=O)C2CCC(O)CC2)c1] | (E)-Isopropyl 3-(3-(N-((1-(4- (dimethylamino)phenyl)piperidin-4-yl)methyl)-4- hydroxycyclohexanecarboxamido)phenyl)acrylate |
| 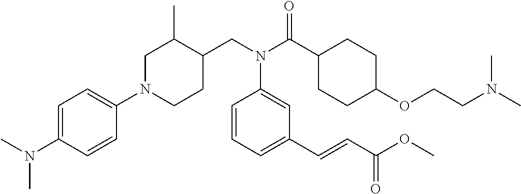 | (E)-Methyl 3-(3-(4-(2-(dimethylamino)ethoxy)-N- ((1-(4-(dimethylamino)phenyl)-3-methylpiperidin-4- yl)methyl)cyclohexanecarboxamido)phenyl)acrylate |
| 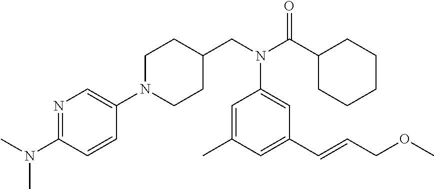 | (E)-N-((1-(6-(Dimethylamino)pyridin-3-yl)piperidin- 4-yl)methyl)-N-(3-(3-methoxyprop-1-en-1-yl)-5- methylphenyl)cyclohexanecarboxamide |
| 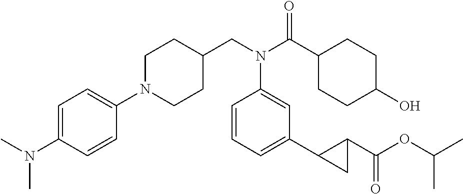 [SMILES: CC(C)OC(=O)C1CC1c1cccc(N(CC2CCN(c3ccc(N(C)C)cc3)CC2)C(=O)C2CCC(O)CC2)c1] | Isopropyl 2-(3-(N-((1-(4- (dimethylamino)phenyl)piperidin-4-yl)methyl)-4- hydroxycyclohexanecarboxamido)phenyl) cyclopropanecarboxylate |
| 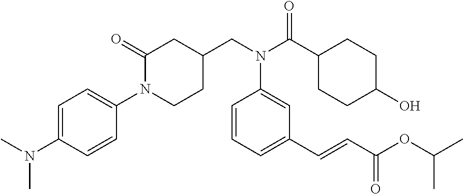 [SMILES: CC(C)OC(=O)/C=C/c1cccc(N(CC2CCN(c3ccc(N(C)C)cc3)C(=O)C2)C(=O)C2CCC(O)CC2)c1] | (E)-Isopropyl 3-(3-(N-((1-(4- (dimethylamino)phenyl)-2-oxopiperidin-4- yl)methyl)-4- hydroxycyclohexanecarboxamido)phenyl)acrylate |
| 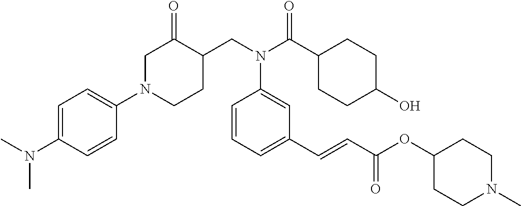 | (E)-1-Methylpiperidin-4-yl 3-(3-(N-((1-(4- (dimethylamino)phenyl)-3-oxopiperidin-4- yl)methyl)-4- hydroxycyclohexanecarboxamido)phenyl)acrylate |
| 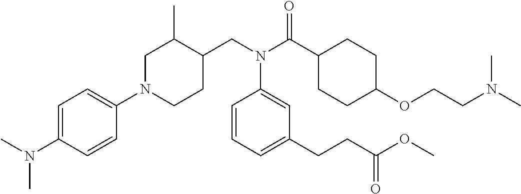 | Methyl 3-(3-(4-(2-(dimethylamino)ethoxy)-N-((1-(4- (dimethylamino)phenyl)-3-methylpiperidin-4- yl)methyl)cyclohexanecarboxamido)phenyl)propanoate |
| 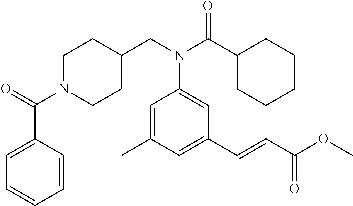 [SMILES: COC(=O)/C=C/c1cc(C)cc(N(CC2CCN(C(=O)c3ccccc3)CC2)C(=O)C2CCCCC2)c1] | (E)-Methyl 3-(3-(N-((1-benzoylpiperidin-4- yl)methyl)cyclohexanecarboxamido)-5- methylphenyl)acrylate |
| 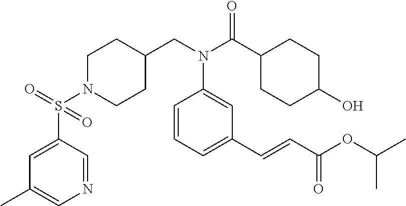 [SMILES: Cc1cncc(S(=O)(=O)N2CCC(CN(C(=O)C3CCC(O)CC3)c3cccc(/C=C/C(=O)OC(C)C)c3)CC2)c1] | (E)-Isopropyl 3-(3-(4-hydroxy-N-((1-((5- methylpyridin-3-yl)sulfonyl)piperidin-4- yl)methyl)cyclohexanecarboxamido)phenyl)acrylate |
| 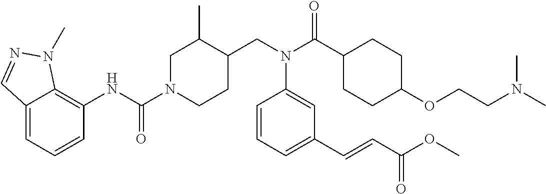 [SMILES: COC(=O)/C=C/c1cccc(N(CC2CCN(C(=O)Nc3cccc4cnn(C)c34)CC2C)C(=O)C2CCC(OCCN(C)C)CC2)c1] | (E)-Methyl 3-(3-(4-(2-(dimethylamino)ethoxy)-N- ((3-methyl-1-((1-methyl-1H-indazol-7- yl)carbamoyl)piperidin-4- yl)methyl)cyclohexanecarboxamido)phenyl)acrylate |
| 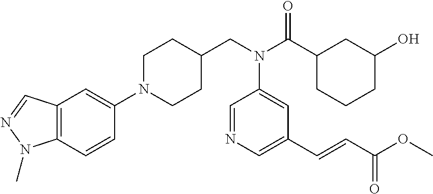 [SMILES: COC(=O)/C=C/c1cncc(N(CC2CCN(c3ccc4c(cnn4C)c3)CC2)C(=O)C2CCCC(O)C2)c1] | (E)-Methyl 3-(5-(3-hydroxy-N-((1-(1-methyl-1H- indazol-5-yl)piperidin-4- yl)methyl)cyclohexanecarboxamido)pyridin-3- yl)acrylate |
| 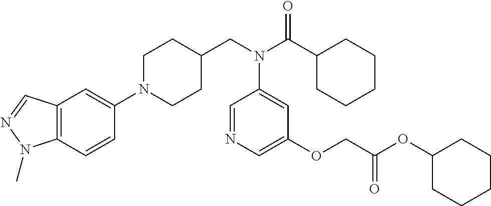 [SMILES: Cn1ncc2cc(N3CCC(CN(C(=O)C4CCCCC4)c4cncc(OCC(=O)OC5CCCCC5)c4)CC3)ccc21] | Cyclohexyl 2-((5-(N-((1-(1-methyl-1H-indazol-5- yl)piperidin-4- yl)methyl)cyclohexanecarboxamido)pyridin-3- yl)oxy)acetate |
| 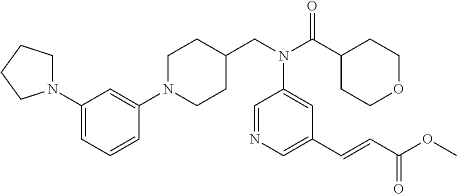 [SMILES: COC(=O)/C=C/c1cncc(N(CC2CCN(c3cccc(N4CCCC4)c3)CC2)C(=O)C2CCOCC2)c1] | (E)-Methyl 3-(5-(N-((1-(3-(pyrrolidin-1- yl)phenyl)piperidin-4-yl)methyl)tetrahydro-2H- pyran-4-carboxamido)pyridin-3-yl)acrylate |
| 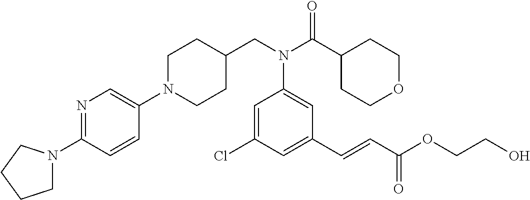 [SMILES: O=C(/C=C/c1cc(Cl)cc(N(CC2CCN(c3ccc(N4CCCC4)nc3)CC2)C(=O)C2CCOCC2)c1)OCCO] | (E)-2-Hydroxyethyl 3-(3-chloro-5-(N-((1-(6- (pyrrolidin-1-yl)pyridin-3-yl)piperidin-4- yl)methyl)tetrahydro-2H-pyran-4- carboxamido)phenyl)acrylate |
| 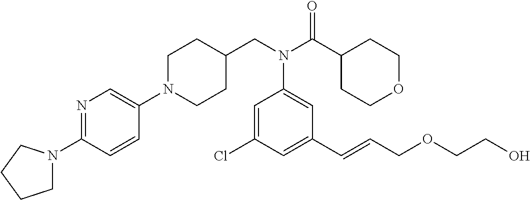 [SMILES: O=C(C1CCOCC1)N(CC1CCN(c2ccc(N3CCCC3)nc2)CC1)c1cc(Cl)cc(/C=C/COCCO)c1] | (E)-N-(3-Chloro-5-(3-(2-hydroxyethoxy)prop-1-en-1- yl)phenyl)-N-((1-(6-(pyrrolidin-1-yl)pyridin-3- yl)piperidin-4-yl)methyl)tetrahydro-2H-pyran-4- carboxamide |
| 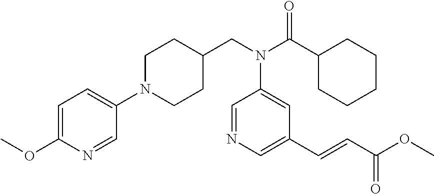 | (E)-Methyl 3-(5-(N-((1-(6-methoxypyridin-3- yl)piperidin-4- yl)methyl)cyclohexanecarboxamido)pyridin-3- yl)acrylate |
| 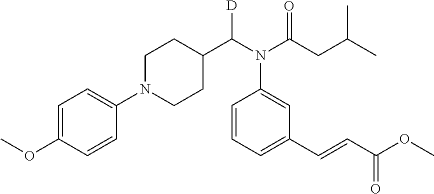 | (E)-Methyl 3-(3-(N-(deutero(1-(4- methoxyphenyl)piperidin-4-yl)methyl)-3- methylbutanamido)phenyl)acrylate |
| 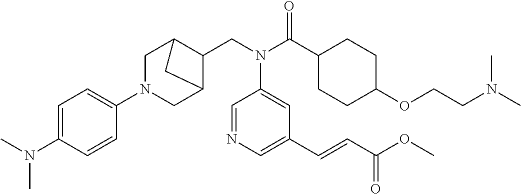 | (E)-Methyl 3-(5-(4-(2-(dimethylamino)ethoxy)-N- ((3-(4-(dimethylamino)phenyl)-3- azabicyclo[3.1.1]heptan-6- yl)methyl)cyclohexanecarboxamido)pyridin-3- yl)acrylate |
| 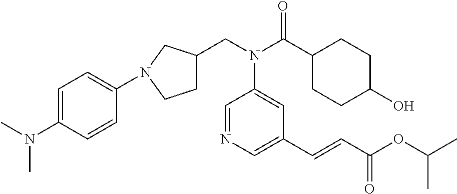 [SMILES: CC(C)OC(=O)/C=C/c1cncc(N(CC2CCN(c3ccc(N(C)C)cc3)C2)C(=O)C2CCC(O)CC2)c1] | (E)-Isopropyl 3-(5-(N-((1-(4- (dimethylamino)phenyl)pyrrolidin-3-yl)methyl)-4- hydroxycyclohexanecarboxamido)pyridin-3- yl)acrylate |
| 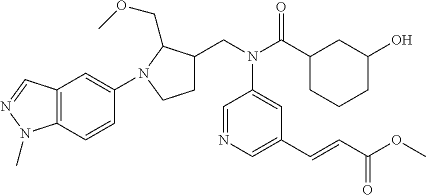 [SMILES: COCC1C(CN(C(=O)C2CCCC(O)C2)c2cncc(/C=C/C(=O)OC)c2)CCN1c1ccc2c(cnn2C)c1] | (E)-Methyl 3-(5-(3-hydroxy-N-((2-(methoxymethyl)- 1-(1-methyl-1H-indazol-5-yl)pyrrolidin-3- yl)methyl)cyclohexanecarboxamido)pyridin-3- yl)acrylate |
| 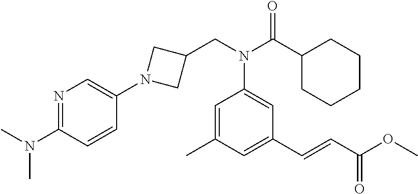 | (E)-Methyl 3-(3-(N-((1-(6-(dimethylamino)pyridin-3- yl)azetidin-3-yl)methyl)cyclohexanecarboxamido)-5- methylphenyl)acrylate |
| 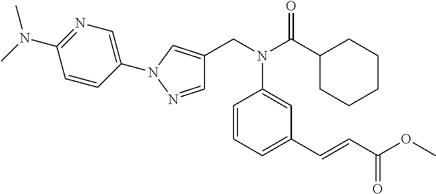 | (E)-Methyl 3-(3-(N-((1-(6-(dimethylamino)pyridin-3- yl)-1H-pyrazol-4- yl)methyl)cyclohexanecarboxamido)phenyl)acrylate |
| 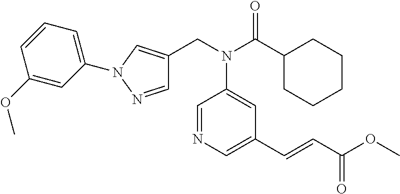 [SMILES: COC(=O)/C=C/c1cncc(N(Cc2cnn(-c3cccc(OC)c3)c2)C(=O)C2CCCCC2)c1] | (E)-Methyl 3-(5-(N-((1-(3-methoxyphenyl)-1H- pyrazol-4- yl)methyl)cyclohexanecarboxamido)pyridin-3- yl)acrylate |
| 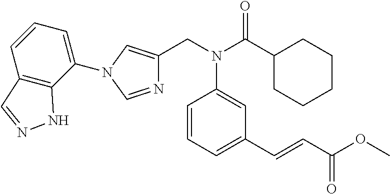 [SMILES: COC(=O)/C=C/c1cccc(N(Cc2cn(-c3cccc4cn[nH]c34)cn2)C(=O)C2CCCCC2)c1] | (E)-Methyl 3-(3-(N-((1-(1H-indazol-7-yl)-1H- imidazol-4- yl)methyl)cyclohexanecarboxamido)phenyl)acrylate |
| 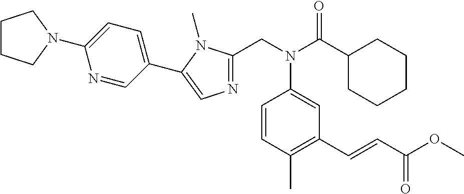 [SMILES: COC(=O)/C=C/c1cc(N(Cc2ncc(-c3ccc(N4CCCC4)nc3)n2C)C(=O)C2CCCCC2)ccc1C] | (E)-Methyl 3-(2-methyl-5-(N-((1-methyl-5-(6- (pyrrolidin-1-yl)pyridin-3-yl)-1H-imidazol-2- yl)methyl)cyclohexanecarboxamido)phenyl)acrylate |
| 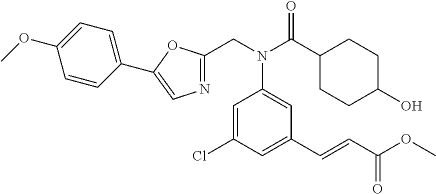 | (E)-Methyl 3-(3-chloro-5-(4-hydroxy-N-((5-(4- methoxyphenyl)oxazol-2- yl)methyl)cyclohexanecarboxamido)phenyl)acrylate |
| 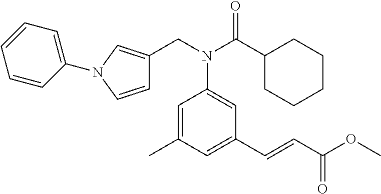 [SMILES: COC(=O)/C=C/c1cc(C)cc(N(Cc2ccn(-c3ccccc3)c2)C(=O)C2CCCCC2)c1] | (E)-Methyl 3-(3-methyl-5-(N-((1-phenyl-1H-pyrrol- 3- yl)methyl)cyclohexanecarboxamido)phenyl)acrylate |
| 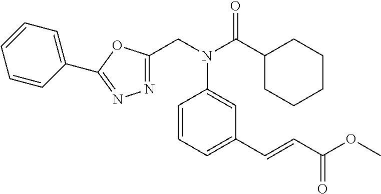 [SMILES: COC(=O)/C=C/c1cccc(N(Cc2nnc(-c3ccccc3)o2)C(=O)C2CCCCC2)c1] | (E)-Methyl 3-(3-(N-((5-phenyl-1,3,4-oxadiazol-2- yl)methyl)cyclohexanecarboxamido)phenyl)acrylate |
| 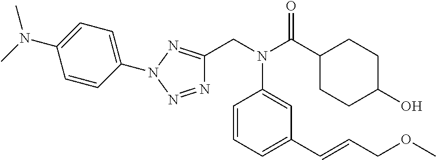 | (E)-N-((2-(4-(Dimethylamino)phenyl)-2H-tetrazol-5- yl)methyl)-4-hydroxy-N-(3-(3-methoxyprop-1-en-1- yl)phenyl)cyclohexanecarboxamide |
| 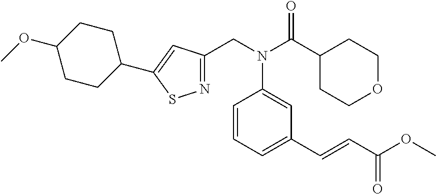 | (E)-Methyl 3-(3-(N-((5-(4- methoxycyclohexyl)isothiazol-3- yl)methyl)tetrahydro-2H-pyran-4- carboxamido)phenyl)acrylate |
| 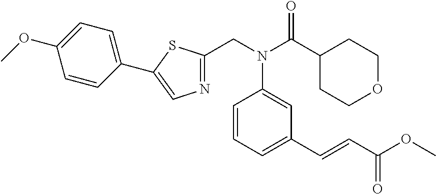 | (E)-Methyl 3-(3-(N-((5-(4-methoxyphenyl)thiazol-2- yl)methyl)tetrahydro-2H-pyran-4- carboxamido)phenyl)acrylate |
| 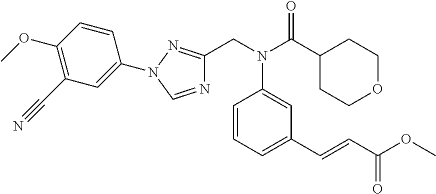 | (E)-Methyl 3-(3-(N-((1-(3-cyano-4-methoxyphenyl)- 1H-1,2,4-triazol-3-yl)methyl)tetrahydro-2H-pyran-4- carboxamido)phenyl)acrylate |
| 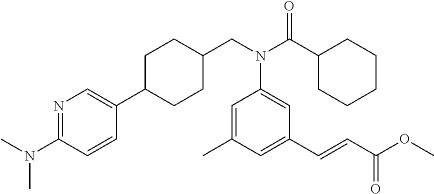 | (E)-Methyl 3-(3-(N-((4-(6-(dimethylamino)pyridin-3- yl)cyclohexyl)methyl)cyclohexanecarboxamido)-5- methylphenyl)acrylate |
| 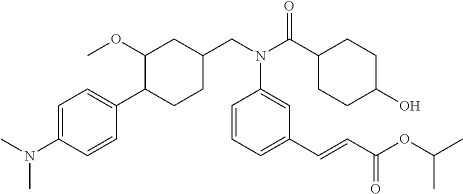 [SMILES: COC1CC(CN(C(=O)C2CCC(O)CC2)c2cccc(/C=C/C(=O)OC(C)C)c2)CCC1c1ccc(N(C)C)cc1] | (E)-Isopropyl 3-(3-(N-((4-(4- (dimethylamino)phenyl)-3- methoxycyclohexyl)methyl)-4- hydroxycyclohexanecarboxamido)phenyl)acrylate |
| 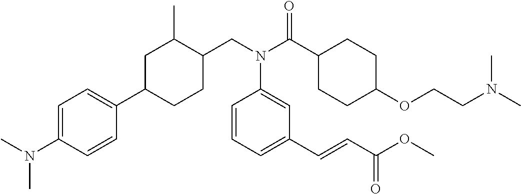 | (E)-Methyl 3-(3-(4-(2-(dimethylamino)ethoxy)-N- ((4-(4-(dimethylamino)phenyl)-2- methylcyclohexyl)methyl)cyclohexanecarboxamido) phenyl)acrylate |
| 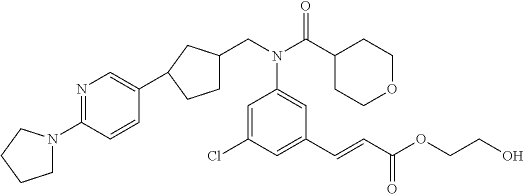 [SMILES: O=C(/C=C/c1cc(Cl)cc(N(CC2CCC(c3ccc(N4CCCC4)nc3)C2)C(=O)C2CCOCC2)c1)OCCO] | (E)-2-Hydroxyethyl 3-(3-chloro-5-(N-((3-(6- (pyrrolidin-1-yl)pyridin-3- yl)cyclopentyl)methyl)tetrahydro-2H-pyran-4- carboxamido)phenyl)acrylate |
| 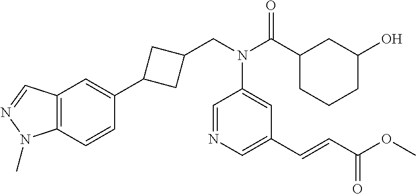 [SMILES: COC(=O)/C=C/c1cncc(N(CC2CC(c3ccc4c(cnn4C)c3)C2)C(=O)C2CCCC(O)C2)c1] | (E)-Methyl 3-(5-(3-hydroxy-N-((3-(1-methyl-1H- indazol-5- yl)cyclobutyl)methyl)cyclohexanecarboxamido) pyridin-3-yl)acrylate |

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid), caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (-L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base to provide a “pharmaceutically acceptable base addition salt.”. In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of/V-oxides (if appropriate), crystalline forms (also known as polymorphs), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. The prodrug may be a substrate for a transporter. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiffbases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

In some embodiments, the compounds described herein are rapidly metabolized following absorption from the gastro-intestinal tract to metabolites that have greatly reduced FXR agonist activity.

In additional or further embodiments, the compounds are rapidly metabolized in plasma.

In additional or further embodiments, the compounds are rapidly metabolized by the intestines.

In additional or further embodiments, the compounds are rapidly metabolized by the liver.

### Synthesis of Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference bodes and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes, and “Chemistry of Functional Groups” John Wiley & Sons, in 73 volumes.

The compounds described herein are prepared by the general synthetic routes described below in Schemes 1-3.

In some embodiments, compounds described herein are prepared as outlined in Scheme 1.

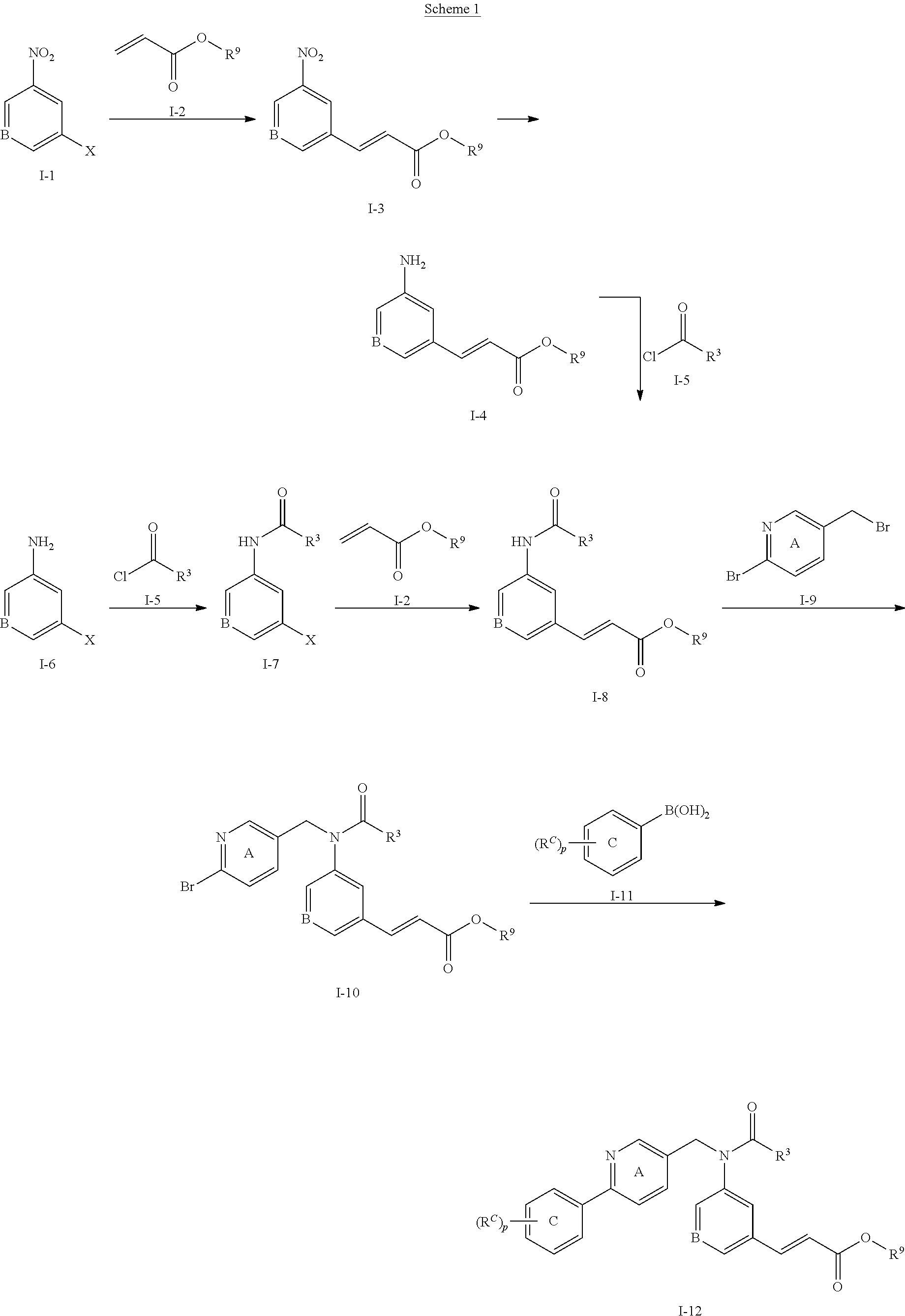

In Scheme 1, rings A and C are as described herein. In some embodiments, B is —CH. In some embodiments, R³ is cyclohexyl. In some embodiments, R⁹ is methyl. In some embodiments, R^{C} is —N(R¹⁰)₂ and p is 1. In some embodiments, R¹⁰ is methyl.

A compound of general structure I-12 may be prepared in variety of ways. In some embodiments, as shown in Scheme 1, a compound of general structure I-3 is prepared from the palladium-catalyzed Heck reaction of halide I-1 with acrylate I-2. In some embodiments, X is Br or I. Suitable conditions for palladium-catalyzed Heck reaction include Pd₂(dba)₃ and P(o-tolyl)₃ with a suitable base and solvent at an appropriate temperature for an appropriate amount of time. In some instances, the suitable base is TEA. In some instances, the suitable solvent is DMF. In some instances, the appropriate temperature is about 90° C. In some instances, the appropriate reaction time is about 12 h to about 24 h. In some instances, I-3 is subjected under transition-metal reduction conditions to provide amine I-4. Suitable reaction conditions for transition-metal reduction include but are not limited to Fe, NH₄Cl in a suitable solvent mixture at an appropriate temperature for an appropriate amount of time. In some embodiments, the suitable solvent is EtOH and H₂O. In some embodiments, the appropriate temperature is about 105° C. and the appropriate time is about 1 h. In other instances, the appropriate temperature is about 90° C. and the appropriate time is about 12 h. In some embodiments, suitable reaction conditions for transition-metal reduction include SnCl₂ in a suitable solvent, such as MeOH, for an appropriate amount of time and at an appropriate temperature. In some embodiments, the appropriate temperature is about 65° C. and the appropriate time is about 2 h. In some embodiments, further reaction of I-4 with acyl chloride I-5 under N-acylation conditions provides compound I-8. Suitable N-acylation conditions include but are not limited to the use of a suitable base, such as TEA in a suitable solvent, such as DCM, for an appropriate time and at an appropriate temperature. In some embodiments, the appropriate time and temperature is about 1 h and about 0° C. Other suitable conditions include TEA and DMAP in a suitable solvent, such as DCM, for an appropriate time and at an appropriate temperature. In some embodiments, the appropriate time and temperature is about 14 h and about 0° C. to rt.

Alternatively, compound I-8 is prepared from amine I-6. In some embodiments, acylation of amine I-6 with acyl chloride I-5 affords compound I-7. In some embodiments, X is Br or I. Suitable acylation conditions include but are not limited to the use of a suitable base, such as TEA in a suitable solvent, such as DCM, for an appropriate time and at an appropriate temperature. In some embodiments, the appropriate time and temperature is about 1 h and about 0° C. Other suitable conditions include TEA and DMAP in a suitable solvent, such as DCM, for an appropriate amount time and at an appropriate temperature. In some embodiments, the appropriate time and temperature is about 14 h and about 0° C. to rt after the addition of acyl chloride I-5. In some embodiments, compound I-8 is prepared from the palladium-catalyzed Heck cross-coupling of bromide I-7 with acrylate I-2. Suitable conditions for palladium-catalyzed Heck cross-coupling include Pd₂(dba)₃ and P(o-tdyl)₃ with a suitable base and solvent at an appropriate temperature for an appropriate amount of time. In some instances, the suitable base is TEA. In some instances, the suitable solvent is DMF. In some instances, the appropriate temperature is about 90° C. In some instances, the appropriate reaction time is about 12 h to about 24 h. In some embodiments, acrylate I-2 and amine I-6 are coupled under under palladium-catalyzed Heck reaction conditions as described above to provide amine I-4.

In some embodiments, compound I-10 is prepared from the N-alkylation of I-8 with benzyl bromide I-9 with a suitable base and suitable solvent, such as THF or DMF, at a suitable temperature for a suitable amount of time. In some embodiments, the suitable base is NaH. In some embodiments, the compound I-8 is pretreated with the suitable base for an appropriate amount of time, such as about 0.5 h, before the addition of benzyl bromide I-9. In some embodiments, the appropriate time and temperature is about 12 h and about 0° C. to rt after the addition of benzyl bromide I-9. In some embodiments, compound I-12 is prepared from the palladium-catalyzed cross coupling of bromide I-10 with boronic acid I-11. Suitable palladium catalysts for cross-coupling include but are not limited to Pd(PPh₃)₄ and Pd(dppf)Cl₂ in a suitable solvent, such as DMF, with an appropriate base at the suitable temperature for an appropriate amount of time. In some embodiments, the appropriate base is Cs₂CO₃. In some embodiments, the suitable temperature is about 90° C. In some embodiments, the appropriate amount of time is about 12 h.

In some embodiments, compounds described herein are prepared as outlined in Scheme 2.

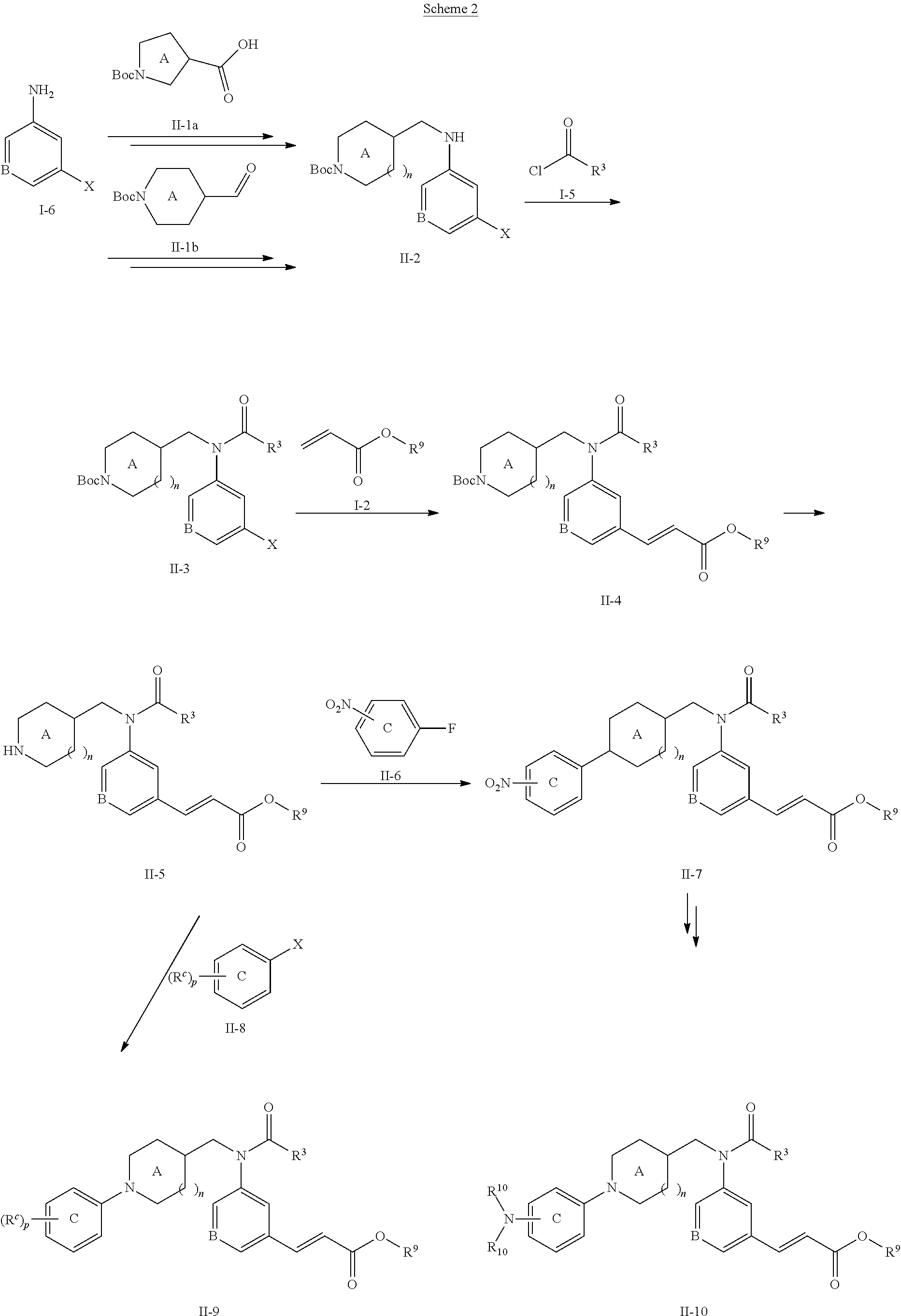

In Scheme 2, rings A and C are as described herein. In some embodiments, B is —CH. In some embodiments, R³ is cyclohexyl. In some embodiments, R⁹ is methyl. In some embodiments, R^{C} is —N(R¹⁰)₂ and p is 1. In some embodiments, R¹⁰ is methyl. In some embodiments, X is I, Br, or Cl. In some embodiments, X is I.

Compound of general structures II-9 and II-10 may be prepared in variety of ways. As shown in Scheme 2, compound II-2 is prepared from compound I-6 In some embodiments, compound I-6 is treated with acid II-1a to provide an amide that is then further subjected to amide reduction conditions to provide compound II-2. Appropriate reaction conditions for amide formation include a suitable coupling agent, such as HATU, in the presence of a suitable base in a suitable solvent, such as DMF at a suitable temperature for a suitable amount of time. In some embodiments, the suitable base is i-Pr₂NEt. In some embodiments, compound II-1a is pre-treated with the suitable coupling agent and amine base at a suitable temperature prior to addition of compound I-6 In some embodiments, compound I-6 is added about 0.5 h after pre-treating compound II-1a with the coupling agent and the amine base. In some embodiments, the suitable reaction time is about 15 h and the suitable temperature is about 0° C. to rt. Appropriate amide reductive conditions include but are not limited to BH₃—SMe₂ in an appropriate solvent, such as THF at an appropriate temperature and time, such as about 90° C. for about 12 h.

In other embodiments, I-6 is treated with aldehyde II-1b is subjected under reductive amination conditions to provide compound II-2. Appropriate reaction conditions for reductive amination includes but are not limited to a first step of using AcOH with a suitable solvent, such as MeOH, for an appropriate amount of time and temperature, such as for about 2 h at rt. Appropriate conditions for subsequent reduction include but are not limited to NaBH₃CN in an appropriate solvent at an appropriate temperature and for an appropriate time, such as at rt for about 16 h.

In some embodiments, compound II-2 is subjected with acyl chloride I-5 to provide II-3. Suitable reaction conditions for this step include but are not limited to a suitable base in an appropriate solvent at a suitable temperature, such as about 0° C. to rt, for an appropriate amount of time, such as about 5 h. In some embodiments, the suitable base is TEA. In some embodiments, the appropriate solvent is DCM. In some embodiments, a palladium-catalyzed Heck reaction of bromide II-3 with acrylate I-2 provides compound II-4. Suitable conditions for palladium-catalyzed Heck reaction include Pd₂dba₃ and P(o-tolyl)₃ with a suitable base and solvent at an appropriate temperature for an appropriate amount of time. In some instances, the suitable base is TEA. In some instances, the suitable solvent is DMF. In some instances, the appropriate temperature is about 90° C. In some instances, the appropriate reaction time is about 6 h. In some embodiments, exposure of compound II-4 under acidic conditions provides compound II-5. Examples of suitable acidic conditions include but are not limited to TFA in a suitable solvent, such as DCM, at a suitable temperature for an appropriate amount of time. In some instances, the suitable temperature is rt and the appropriate amount of time is about 2 h. In some instances, compound II-5 is subjected to palladium-catalyzed cross-coupling conditions with aryl halide II-8 to afford compound II-9. Suitable palladium catalysts for cross-coupling include but are not limited to Pd(OAc)₂ with a suitable ligand in a suitable solvent, such as PhMe, with an appropriate base at the suitable temperature for an appropriate amount of time. In some embodiment, the suitable ligand is BINAP. In some embodiments, the appropriate base is Cs₂CO₃. In some embodiments, the suitable temperature is about 100° C. In some embodiments, the appropriate amount of time is about 7 h.

Alternatively, compound II-5 is subjected under nucleophilic aromatic substitution (S_{N}Ar) conditions with nitro-substituted aryl fluoride II-6 to provide compound II-7. Suitable nucleophilic aromatic substitution conditions include but are not limited to a suitable base in the appropriate solvent, such as DMF, at the appropriate temperature for a suitable amount of time. In some embodiments, the suitable base is K₂CO₃. In some embodiments, the suitable temperature is about 80° C. In some embodiments, the appropriate amount of time is about 6 h. In some embodiments, compound II-7 is subjected under reaction conditions to facilitate the reduction of the nitro group to provide an amine compound that is then subjected under N-alkylation conditions to afford compound II-10. Suitable reaction conditions for reducing a nitro group to the amine include but are not limited to Fe in the presence of NH₄Cl, EtOH/H₂O at a suitable temperature for an appropriate amount of time. Appropriate N-alkylation conditions include but are not limited to a two-step treatment that employs CH₂O with AcOH in a suitable solvent, such as MeOH, for a suitable amount of time at an appropriate temperature, such as about 3 h at rt, for the first step and a reducing agent, such as NaBH₃CN, for a suitable time at an appropriate temperature, such as about 12 h at rt, for the second step.

In some embodiments, compounds described herein are prepared as outlined in Scheme 3.

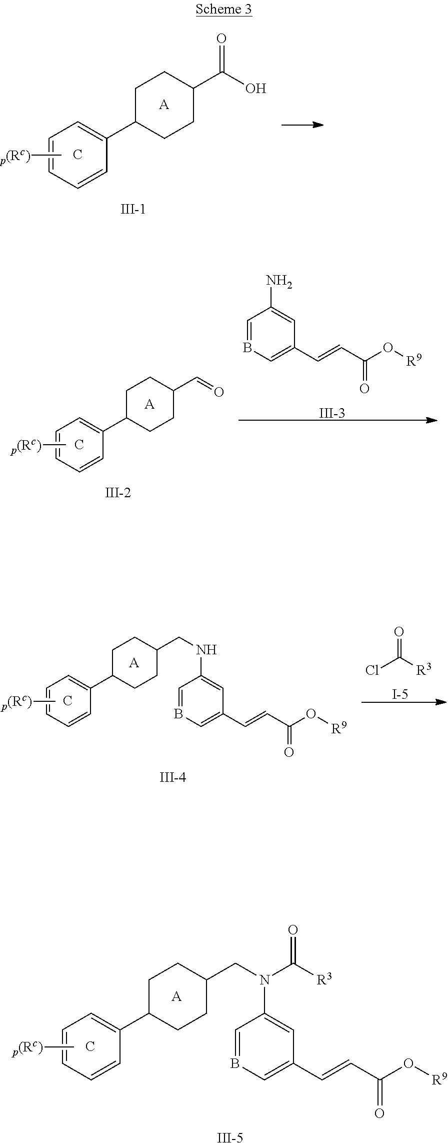

In Scheme 3, rings A and C are as described herein. In some embodiments, B is —CH. In some embodiments, R³ is cyclohexyl. In some embodiments, R⁹ is methyl. In some embodiments, R^{C} is —Cl and p is 1.

In some embodiments, acid III-I is transformed into aldehyde III-2. In some instances, acid III-I is subjected under reductive conditions to provide an alcohol product that is then oxidized to provide aldehyde III-2. Suitable reductive conditions include but are not limited to BH₃—SMe₂ in a suitable solvent, such as THF, at an appropriate temperature for an appropriate amount of time, such as at rt to about 90° C. for about 1.5 h. Suitable oxidizing agents include but are not limited to PCC. In some embodiments, the oxidizing agent is employed with a suitable solvent, such as DCM, at an appropriate temperature for a suitable amount of time, such as at rt for about 2 h. In some embodiments, amine III-4 is obtained from subjecting aldehyde III-2 and aniline III-3 under reductive amination conditions. In some embodiments, aldehyde III-2 and aniline III-3 are first treated under acidic conditions, such as AcOH in MeOH at rt for about 1 h, following by treatment with a reducing agent, such as NaBH₃CN, for a suitable time at an appropriate temperature, such as rt for about 12 h. In some embodiments, acylation of amine III-4 with acyl chloride 1-5 affords compound III-5. Suitable acylation conditions include but are not limited to TEA in a suitable solvent, such as DCM, for an appropriate time and at an appropriate temperature, such as about 0° C. for about 2 h then rt for about 3 h.

In some embodiments, intermediates used in the preparation of compounds described herein are prepared as outlined in Scheme 4.

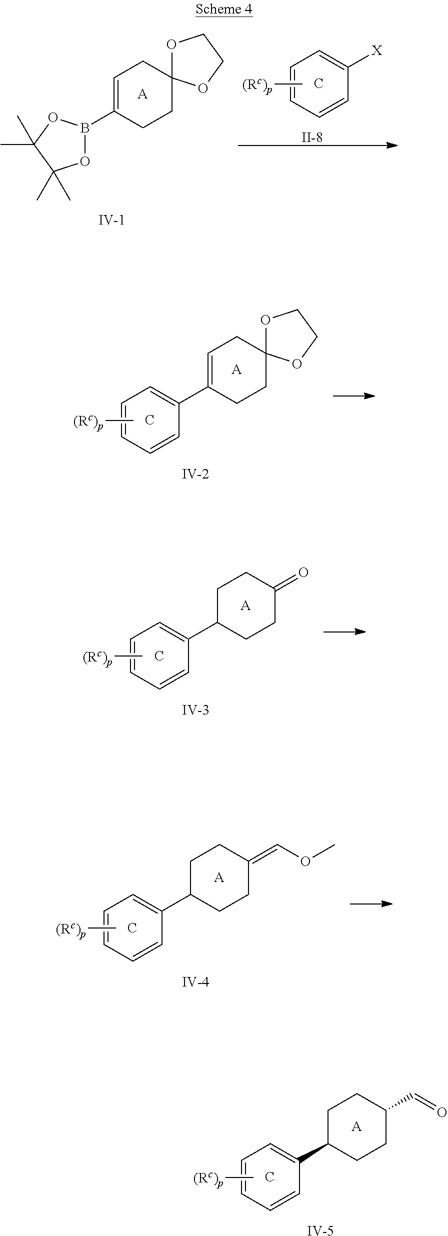

In Scheme 4, rings A and C are as described herein. In some embodiments, p is 2 and each R^{C} is independently selected from —OR¹⁰ and substituted or unsubstituted C₁-C₆alkyl. In some embodiments, p is 2 and each R^{C} is independently selected from —OR¹⁰ and methyl. In some embodiments, R¹⁰ is methyl. In some embodiments, X is I, Br, or Cl. In some embodiments, X is I.

In some embodiments, boronic ester IV-1 is reacted with halide II-8 under suitable palladium-catalyzed cross-coupling reaction conditions to provide IV-2 In some embodiments, suitable palladium-catalyzed cross-coupling reaction conditions include Pd(dppf)Cl₂ with an appropriate base, such as 1M Na₂CO₃, with an appropriate solvent for an appropriate time and at an appropriate temperature. In some embodiments, the appropriate solvent is dioxane. In some embodiments, the appropriate time and appropriate temperature is 2.5 hours at 50° C. In some embodiments, IV-2 is subjected under suitable palladium-catalyzed hydrogenation conditions followed by treatment under appropriate acidic conditions to provide cyclohexanone IV-3. In some embodiments, suitable palladium-catalyzed hydrogenation conditions include 10% Pd/C with hydrogen (1 atm) in a suitable solvent, such as EtOAc, for an appropriate amount of time at an appropriate temperature. In some embodiments, the appropriate amount of time is 4.5 hours at room temperature. In some embodiments, appropriate acidic conditions include formic acid in water and toluene for a suitable amount of time at an appropriate temperature. In some embodiments, the suitable amount of time at an appropriate temperature is 4 hours at 120° C. In some embodiments, IV-3 is reacted with under suitable one carbon-homologation conditions to provide IV-4. In some embodiments, suitable one-carbon-homologation conditions, includes pre-treating (methoxymethyl)triphenyl phosphonium chloride [Ph₃P⁺CH₂OCH₃Cl⁻] with an appropriate base, such as NaHMDS, with an appropriate solvent for an appropriate amount of time at an appropriate temperature before the addition of cyclohexanone IV-3. In some embodiments, the appropriate solvent is THF. In some embodiments, the appropriate amount of time at an appropriate temperature is 30 mins at 0° C. In some embodiments, after IV-3 is added the reaction is continued for another 30 mins at 0° C. In some embodiments, IV-4 is then subjected under suitable acidic conditions to provide a mixture of cis and trans aldehydes IV-5 In some embodiments, suitable acidic conditions include formic acid in water/toluene at 120° C. for about 2 hours. In some embodiments, further subjection of aldehyde IV-5 under appropriate basic conditions provides a mostly trans aldehyde IV-5. In some embodiments, appropriate basic conditions include NaOH in a suitable solvent mixture, such as EtOH and PhMe, for an appropriate amount of time at an appropriate temperature. In some embodiments, the appropriate amount of time at an appropriate temperature is 5.5 hours at room temperature. In some embodiments, further purification via crystallization or chromatography provides pure trans aldehyde IV-5.

In some embodiments, compounds are prepared as described in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, iso-propyl, butyl, iso-butyl, sec-butyl, t-butyl, pentyl, neopentyl, or hexyl.

An “alkylene” group refers refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to an (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Polycyclic cycloalkyls include, for example, adamantyl, norbornyl (i.e., bicyclo[2.2.1]heptanyl), norbornenyl, decalinyl, 7,7-dimethyl-bicyclo[2.2.1]heptanyl, and the like

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-enyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle are aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(CᵣC₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L, Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuff's or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient, and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

It should be understood that in addition to the ingredients particularly mentioned above, the compounds and compositions described herein may include other agents conventional in the art having regard to the type of formulation in question, for example those suitable for oral administration may include flavoring agents.

Methods of Dosing and Treatment Regimens

In one embodiment, the compounds described herein, or a pharmaceutically acceptable salt thereof, are used in the preparation of medicaments for the treatment of diseases or conditions in a mammal that would benefit from administration of a FXR agonist. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

Disclosed herein, are methods of administering a FXR agonist in combination with an additional therapeutic agent. In some embodiments, the additional therapeutic agent comprises a therapeutic agent for treatment of diabetes or diabetes related disorder or conditions, alcoholic or non-alcoholic liver disease, inflammation related intestinal conditions, or cell proliferative disorders.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion, the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon die dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours, (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient may be additive of the two therapeutic agents or the patient may experience a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, a FXR agonist is administered in combination with an additional therapeutic agent for the treatment of diabetes or diabetes related disorder or conditions.

In some instances, the additional therapeutic agent comprises a statin, an insulin sensitizing drug, an insulin secretagogue, an alpha-glucosidase inhibitor, a GLP agonist, a DPP-4 inhibitor (such as sitagliptin, vildagliptin, saxagliptin, linagliptin, anaglptin, teneligliptin, alogliptin, gemiglptin, or dutoglpitin), a catecholamine (such as epinephrine, norepinephrine, or dopamine), peroxisome proliferator-activated receptor (PPAR)-gamma agonist (e.g., a thiazolidinedione (TZD) [such as pioglitazone, rosiglitazone, rivoglitazone, or troglitazone], aleglitazar, farglitazar, muraglitazar, or tesaglitazar), or a combination thereof. In some cases, the statin is a HMG-CoA reductase inhibitor. In other instances, additional therapeutic agents include fish oil, fibrate, vitamins such as niacin, retinoic acid (e.g., 9 cis-retinoic acid), nicotinamide ribonucleoside or its analogs thereof, or combinations thereof. In some instances, nicotinamide ribonucleoside or its analogs thereof, which promote NAD⁺ production, a substrate for many enzymatic reactions including p450s which is a target for FXR (e.g., see Yang et al., J. Med. Chem. 50:6458-61, 2007).

In some embodiments, a FXR agonist is administered in combination with an additional therapeutic agent such as a statin, an insulin sensitizing drug, an insulin secretagogue, an alpha-glucosidase inhibitor, a GLP agonist, a DPP-4 inhibitor (such as sitagliptin, vildagliptin, saxagliptin, linagliptin, anaglptin, teneligliptin, alogliptin, gemiglptin, or dutoglpitin), a catecholamine (such as epinephrine, norepinephrine, or dopamine), peroxisome proliferator-activated receptor (PPAR)-gamma agonist (e.g., a thiazolidinedione (TZD) [such as pioglitazone, rosiglitazone, rivoglitazone, or troglitazone], aleglitazar, farglitazar, muraglitazar, or tesaglitazar), or combinations thereof, for the treatment of diabetes or diabetes related disorder or conditions. In some embodiments, a FXR agonist is administered in combination with an additional therapeutic agent such as fish oil, fibrate, vitamins such as niacin, retinoic acid (e.g., 9 cis-retinoic acid), nicotinamide ribonucleoside or its analogs thereof, or combinations thereof, for the treatment of diabetes or diabetes related disorder or conditions.

In some embodiments, a FXR agonist is administered in combination with a statin such as a HMG-CoA reductase inhibitor, fish oil, fibrate, niacin or a combination thereof, for the treatment of dyslipidemia.

In additional embodiments, a FXR agonist is administered in combination with a vitamin such as retinene acid for the treatment of diabetes and diabetes related disorder or condition such as lowering elevated body weight and/or lowering elevated blood glucose from food intake.

In some embodiments, the famesoid X receptor agonist is administered with at least one additional therapy. In some embodiments, the at least one additional therapy is a glucose-lowering agent. In some embodiments, the at least one additional therapy is an anti-obesity agent. In some embodiments, the at least one additional therapy is selected from among a peroxisome proliferator activated receptor (PPAR) agonist (gamma, dual, or pan), a dipeptidyl peptidase (IV) inhibitor, a glucagon-like peptide-1 (GLP-I) analog, insulin or an insulin analog, an insulin secretagogue, a sodium glucose co-transporter 2 (SGLT2) inhibitor, a glucophage, a human amylin analog, a biguanide, an alpha-glucosidase inhibitor, a meglitinide, a thiazolidinedione, and sulfonylurea. In some embodiments, the at least one additional therapy is metformin, sitagliptin, saxaglitpin, repaglinide, nateglinide, exenatide, liraglutide, insulin lispro, insulin aspart, insulin glargine, insulin detemir, insulin isophane, and glucagon-like peptide 1, or any combination thereof. In some embodiments, the at least one additional therapy is a lipid-lowering agent. In certain embodiments, the at least one additional therapy is administered at the same time as the famesoid X receptor agonist. In certain embodiments, the at least one additional therapy is administered less frequently than the famesoid X receptor agonist. In certain embodiments, the at least one additional therapy is administered more frequently than the famesoid X receptor agonist. In certain embodiments, the at least one additional therapy is administered prior to administration of the famesoid X receptor agonist. In certain embodiments, the at least one additional therapy is administered after administration of the famesoid X receptor agonist.

In some embodiments, a compound described herein, or a pharmaceutically acceptable salt thereof, is administered in combination with chemotherapy, anti-inflammatory agents, radiation therapy, monoclonal antibodies, or combinations thereof.

In some embodiments, a FXR agonist is administered in combination with an additional therapeutic agent for the treatment of alcoholic or non-alcoholic liver disease. In some embodiments, the additional therapeutic agent includes antioxidant, corticosteroid, anti-tumor necrosis factor (TNF) or a combination thereof.

In some embodiments, a FXR agonist is administered in combination with an additional therapeutic agent such as antioxidant, corticosteroid, anti-tumor necrosis factor (TNF), or a combination thereof, for the treatment of alcoholic or non-alcoholic liver disease. In some embodiments, a FXR agonist is administered in combination with an antioxidant, a vitamin precursor, a corticosteroid, an anti-tumor necrosis factor (TNF), or a combination thereof, for the treatment of alcoholic or non-alcoholic liver disease.

In some embodiments, a FXR agonist is administered in combination with an additional therapeutic agent for the treatment of inflammation related intestinal conditions. In some instances, the additional therapeutic agent comprises an antibiotic (such as metronidazole, vancomycin, and/or fidaxomicin), a corticosteroid, or an additional anti-inflammatory or immuno-modulatory therapy.

In some instances, a FXR agonist is administered in combination with an additional therapeutic agent such as an antibiotic, a corticosteroid, or an additional anti-inflammatory or immuno-modulatory therapy, for the treatment of inflammation related intestinal conditions. In some cases, a FXR agonist is administered in combination with metronidazole, vancomycin, fidaxomicin, corticosteroid, or combinations thereof, for the treatment of inflammation related intestinal conditions.

As discussed above, inflammation is sometimes associated with pseudomembranous colitis. In some instances, pseudomembranous colitis is associated with bacterial overgrowth (such as C. dificile overgrowth). In some embodiments, a FXR agonist is administered in combination with an antibiotic such as metronidazole, vancomycin, fidaxomicin, or a combination thereof, for the treatment of inflammation associated with bacterial overgrowth (e.g., pseudomembranous colitis).

In some embodiments, the FXR agonist is administered in combination with an additional therapeutic agent for the treatment of cell proliferative disorders. In some embodiments, the additional therapeutic agent includes a chemotherapeutic, a biologic (e.g., antibody, for example bevacizumab, cetuximab, or panitumumab), a radiotherapeutic (e.g., FOLFOX, FOLFIRI, CapeOX, 5-FU, leucovorin, regorafenib, irinotecan, or oxaliplatin), or combinations thereof.

In some embodiments, the FXR agonist is administered in combination with an additional therapeutic agent for the treatment of primary biliary cirrhosis. In some embodiments, the additional therapeutic agent includes ursodeoxycholic acid (UDCA).

In some embodiments, a FXR agonist is administered in combination with an additional therapeutic agent such as a chemotherapeutic, a biologic, a radiotherapeutic, or combinations thereof, for the treatment of a cell proliferative disorder. In some instances, a FXR agonist is administered in combination with an antibody (e.g., bevacizumab, cetuximab, or panitumumab), chemotherapeutic, FOLFOX FOLFIRI, CapeOX 5-FU, leucovorin, regorafenib, irinotecan, oxaliplatin, or combinations thereof, for the treatment of a cell proliferative disorder.

### EXAMPLES

The following examples are provided for illustrative purposes only and not to limit the scope of the claims provided herein. As used above, and throughout the description of the invention, the following abbreviations, unless otherwise indicated, shall be understood to have the following meanings:
ACN or MeCN acetonitrile
AcOH acetic acid
BINAP 2,2′-Bis(diphenylphosphino)-1,1′-binaphthalene
Bn benzyl
BOC or Boc tert-butyl carbamate
t-Bu tert-butyl
Cy cyclohexyl
DBA or dba dibenzylideneacetone
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
Dppf or dppf 1,1-bis(diphenylphosphinoferrocene
EEDQ 2-Ethoxy-1-ethoxycarbonyl-1,2-dihydroquinoline
eq equivalents)
Et ethyl
Et₂O diethyl ether
EtOH ethanol
EtOAc ethyl acetate
HATU 1-[bis(dimethylamino)methylene]-1H-1,2,3-triazolo[4,5-b]pyridinium 3-oxid hexafluorophosphate
HPLC high performance liquid chromatographyLAH lithium
aluminum anhydride
LCMS liquid chromatography mass spectrometry
M molar
Me methyl
MeOH methanol
MS mass spectroscopy
MTBE tert-butyl methyl ether
NaHMDS sodium bis(trimethylsilyl)amide
NMM A-methyl-morpholine
NMP A-methyl-pyrrolidin-2-one
NMR nuclear magnetic resonance
PCC pyridinium chlorochromate
i-Pr iso-propyl
Ph phenyl
RP-HPLC reverse phase-high pressure liquid chromatography
TBS tert-butyldimethylsilyl
TFA trifluoroacetic acid
TEA triethylamine
THF tetrahydrofuran
TLC thin layer chromatography

### Intermediate 1

### N-(3-Bromophenyl)cyclohexanecarboxamide

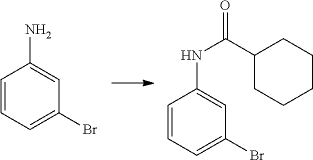 [SMILES: Nc1cccc(Br)c1 ; O=C(Nc1cccc(Br)c1)C1CCCCC1]

Cyclohexanecarbonyl chloride (61.4 g, 419 mmol) was added to a solution of 3-bromoaniline (60 g, 349 mmol), DMAP (4.26 g, 34.9 mmol), and TEA (70.6 g, 698 mmol) in DCM (400 mL) at 0° C. The mixture was warmed to 15° C. slowly, and then stirred for 14 h. Water (200 mL) was added to the reaction, and the mixture was extracted with DCM (3×300 mL). The combined organic phase was washed with brine (2×300 mL), dried over anhydrous Na₂SO₄, filtered and concentrated in vacuo to give a residue. The residue was purified by re-crystallization from MTBE (200 mL) to give N-(3-bromophenyl)cyclohexanecarboxamide (90 g, 85%) as a red brown solid. MS: 282.1 [M+H]⁺.

### Intermediate 2

### (E)-Methyl 3-(3-(cyclohexanecarboxamido)phenyl)acrylate

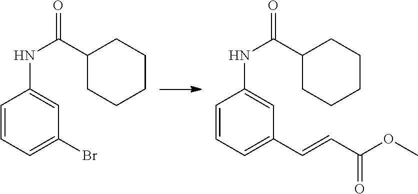 [SMILES: O=C(Nc1cccc(Br)c1)C1CCCCC1 ; COC(=O)/C=C/c1cccc(NC(=O)C2CCCCC2)c1]

A mixture of Intermediate 1 (75 g, 266 mmol), methyl acrylate (91.5 g, 1.06 mol), Pd₂(dba)₃ (24.3 g, 26.6 mmol), tri-o-tolylphosphine (24.3 g, 79.7 mmol), and TEA (135 g, 1.33 mol) in DMF (1.0 L) was degassed with vacuum/nitrogen cycles (3×), stirred at 90° C. for 24 h, filtered, diluted with water (500 mL), and extracted with ethyl acetate (3×1 L). The combined organic layers were washed with water (3×1 L) and brine (2×800 mL), dried over anhydrous Na₂SO₄, filtered and concentrated under reduced pressure to give a residue. The residue was purified by re-crystallization from petroleum ether (1 L) to give (E)-methyl 3-(3-(cyclohexanecarboxamido)phenyl)acrylate (65 g, 82%) as a white solid. ¹H NMR (DMSO-d₆): δ 7.77 (s, 1H), 7.62-7.66 (d, 1H), 7.50-7.52 (d, 1H), 7.30-7.33 (m, 1H), 7.23-7.25 (m, 2H), 6.42-6.46 (d, 1H), 3.79 (s, 3H), 2.23-2.24 (m, 1H), 1.94-1.97 (m, 2H), 1.83-1.85 (m, 2H), 1.70-1.72 (m, 1H), 1.56-1.59 (m, 2H), 1.27-1.33 (m, 3H). MS: 288.2 [M+H]⁺.

The Intermediates below were synthesized from 3-iodoaniline and the appropriate acrylate following the procedure described for Intermediate 2.

| Int | Structure | Name | LCMS |
|---|---|---|---|
| 2.1 | 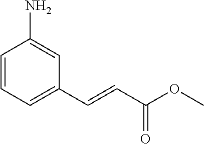 | (E)-Methyl 3-(3- aminophenyl) acrylate | 178.3 |
| 2.2 | 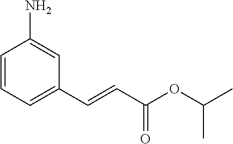 | (E)-Isopropyl 3-(3- aminophenyl) acrylate | 206.1 |

### Intermediate 3

### (E)-tert-Butyl 4-(((3-(3-methoxy-3-oxoprop-1-en-1-yl)phenyl)amino)methyl)piperidine-1-carboxylate

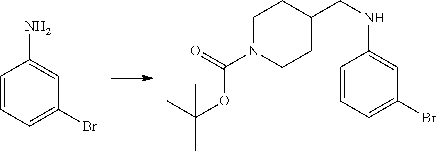 [SMILES: Nc1cccc(Br)c1 ; CC(C)(C)OC(=O)N1CCC(CNc2cccc(Br)c2)CC1]

A mixture of 3-bromoaniline (6.33 mL, 58.1 mmol), acetic acid (1.66 mL, 29.1 mmol), tert-butyl 4-formylpiperidine-1-carboxylate (13.6 g, 64 mmol), and methanol (50 mL) was stirred for 2 h. Sodium cyanoborohydride (7.31 g, 116 mmol) was added to the reaction, and the mixture was stirred for 16 h, quenched with water (300 mL), and then extracted with ethyl acetate (2×400 mL). The combined organic layers were dried over Na₂SO₄, filtered, and concentrated to dryness to give tert-butyl 4-(((3-bromophenyl)amino)methyl)piperidine-1-carboxylate (25 g, 91%) as a purple oil.

### Intermediate 4

### (R)-tert-Butyl 3-(((3-bromophenyl)amino)methyl)pyrrolidine-1-carboxylate

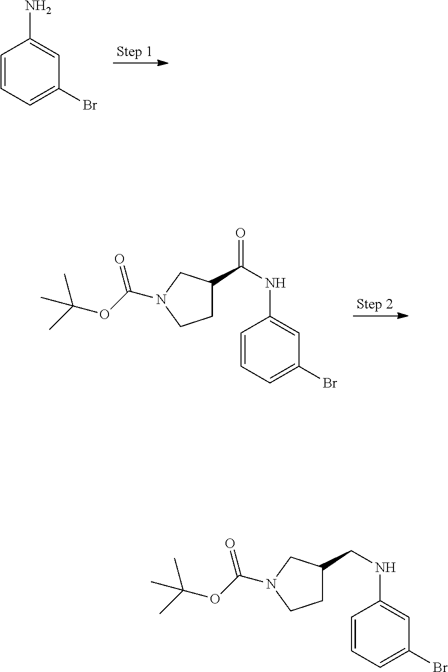

### Step 1: (R)-tert-Butyl 3-((3-bromophenyl)carbamoyl)pyrrolidine-1-carboxylate

A mixture of (R)-1-(tert-butoxycarbonyl)pyrrolidine-3-carboxylic acid (3.8 g, 17.7 mmol), DIPEA (9.2 mL, 53 mmol) and HATU (8.06 g, 21.2 mmol) in DMF (40 mL) was stirred for 30 min at 0° C. 3-bromoaniline (1.92 mL, 17.7 mmol) was added to the reaction, and the solution was stirred for 14.5 h at 30° C. The reaction mixture was quenched by adding water (100 mL), and then extracted with ethyl acetate (2×100 mL). The combined organic layers were washed with brine (200 mL), dried over Na₂SO₄, filtered and concentrated under reduced pressure. The residue was purified by column chromatography (SiO₂, petroleum ether/ethyl acetate=10/1 to 4/1) to yield (R)-tert-butyl 3-((3-bromophenyl)carbamoyl)pyrrolidine-1-carboxylate (6 g, 88%) as a yellow oil. MS: 313.1 [(M−tBu+H)+H]⁺.

### Step 2: (R)-tert-Butyl 3-(((3-bromophenyl)amino)methyl)pyrrolidine-1-carboxylate

To a solution of (R)-tert-butyl 3-((3-bromophenyl)carbamoyl)pyrrolidine-1-carboxylate (6 g, 16 mmol) in THF (40 mL) was added BH₃-Me₂S (10 M, 4.9 mL, 49 mmol). The mixture was degassed with vacuum/nitrogen cycles (3×), and stirred at 90° C. for 12 h. Methanol (100 mL) was added to the reaction at 0° C., and the mixture was heated at reflux for 1 h. The reaction mixture was extracted with ethyl acetate (2×300 mL), and the combined organic layers were washed with brine (500 mL), dried over Na₂SO₄, and concentrated. The residue was purified by column chromatography (SiO₂, petroleum ether/ethyl acetate=50/1 to 5/1) to give (R)-tert-butyl 3-(((3-bromophenyl)amino)methyl)pyrrolidine-1-carboxylate (3.5 g, 59%) as yellow oil.

The Intermediate below was synthesized following the procedures described for Intermediate 4

| Int | Structure | Name | LCMS |
|---|---|---|---|
| 4.1 | 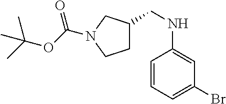 | (S)-tert-Butyl 3-(((3- bromophenyl) amino)methyl) pyrrolidine-1- carboxylate | |

### Intermediate 5

### (E)-Methyl 3-(3-(N-(piperidin-4-ylmethyl)cyclohexanecarboxamido)phenyl)acrylate

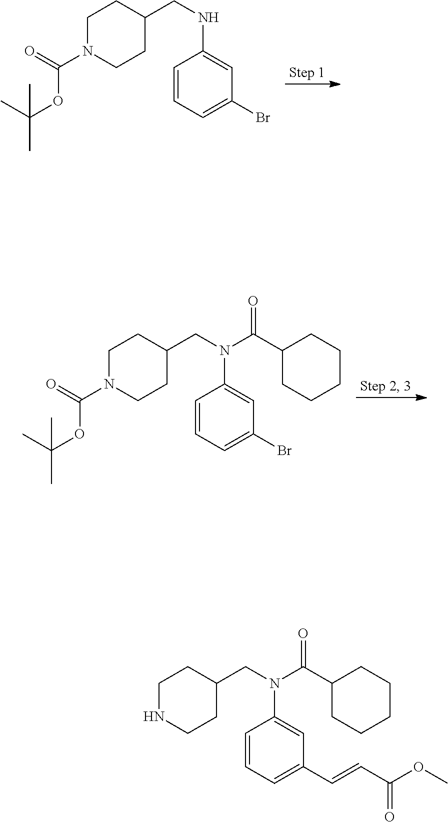 [SMILES: CC(C)(C)OC(=O)N1CCC(CNc2cccc(Br)c2)CC1 ; CC(C)(C)OC(=O)N1CCC(CN(C(=O)C2CCCCC2)c2cccc(Br)c2)CC1 ; COC(=O)/C=C/c1cccc(N(CC2CCNCC2)C(=O)C2CCCCC2)c1]

### Step 1: tert-Butyl 4-((N-(3-bromophenyl)cyclohexanecarboxamido)methyl)piperidine-1-carboxylate

To a solution of Intermediate 3 (25 g, 68 mmol) in DCM (100 mL) was added TEA (56 mL, 406 mmol) slowly at 0° C. The mixture was stirred for 0.5 h, and cyclohexanecarbonyl chloride (27 mL, 203 mmol) was added slowly at 0° C. The resulting mixture was allowed to warm up to 25° C. and stirred for 2.5 h. Water (200 mL) was added to the mixture, and the mixture was extracted with ethyl acetate (2×300 mL). The combined organic layers were washed with brine (500 mL), dried over Na₂SO₄, filtered, and concentrated under reduced pressure. The residue was purified by column chromatography (SiO₂, petroleum ether/ethyl acetate=30/1 to 5/1) to give tertbutyl 4-((N-(3-bromophenyl)cyclohexanecarboxamido)methyl)piperidine-1-carboxylate (21 g, 62%) as a yellow oil. MS: 423.2 [(M−tBu+H)+H]⁺.

### Step 2: (E)-tert-Butyl 4-((N-(3-(3-methoxy-3-oxoprop-1-en-1-yl)phenyl)cyclohexanecarboxamido)methyl)piperidine-1-carboxylate

The mixture of tert-butyl 4-((N-(3-bromophenyl)cyclohexanecarboxamido)methyl)piperidine-1-carboxylate (21 g, 43.8 mmol), methyl acrylate (15.7 mL, 175 mmol), Pd₂(dba)₃ (4.01 g, 4.38 mmol), TEA (18.2 mL, 131 mmol) and tri-o-tolylphosphine (4.0 g, 13 mmol) in DMF (40 mL) was degassed with vacuum/nitrogen cycles (3×), heated at 90° C. for 12 h, and filtered. Water (300 mL) was added to the filtrate, and the mixture was extracted with ethyl acetate (3×200 mL). The combined organic layers were washed with brine (600 mL), dried over Na₂SO₄, filtrated, and concentrated under reduced pressure. The residue was purified by column chromatography (petroleum ether/ethyl acetate=10/1 to 3/1) to give (E)-tert-butyl 4-((N-(3-(3-methoxy-3-oxoprop-1-en-1-yl)phenyl)cyclohexanecarboxamido)methyl)piperidine-1-carboxylate (18 g, 73%) as a yellow oil. ¹H NMR (400 MHz, DMSO-d₆): δ 7.62-7.80 (m, 3H), 7.51 (t, 1H), 7.35 (d, 1H), 6.75 (d, 1H), 3.86 (d, 2H), 3.73 (s, 3H), 3.44-3.60 (m, 2H), 2.57-2.68 (m, 2H), 2.05 (br s, 1H), 1.44-1.67 (m, 8H), 1.28-1.43 (m, 11H), 0.70-1.17 (m, 5H). MS: 429.3 [(M−tBu+H)+H]⁺.

### Step 3: (E)-Methyl 3-(3-(N-(piperidin-4-ylmethyl)cyclohexanecarboxamido)phenyl)acrylate

Trifluoroacetic acid (25 mL, 330 mmol) was added to a solution of (E)-tert-butyl 4-((A^{r}-(3-(3-methoxy-3-oxoprop-1-en-1-yl)phenyl)cyclohexanecarboxamido)methyl) piperidine-1-carboxylate (8.0 g, 16.5 mmol) in DCM (250 mL). The mixture was stirred at 25° C. for 3 h. The pH was adjusted to pH=8 with saturated NaHCO₃ solution, and then the mixture was extracted with DCM (2×300 mL). The combined organic layers were washed with brine (500 mL), dried over Na₂SO₄, and concentrated to give (E)-methyl 3-(3-(N-(piperidin-4-ylmethyl)cyclohexanecarboxamido)phenyl)acrylate (6 g, 83%) as a yellow gum. MS: 385.3 [M+H]⁺.

The Intermediates below were synthesized following the procedures described for Intermediate 5.

| Int | Structure | Name | [M + H]⁺ |
|---|---|---|---|
| 5.1 | 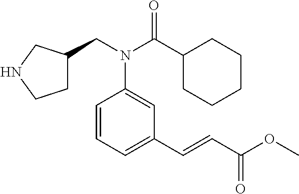 | (R,E)-Methyl 3-(3-(N-(pyrrolidin- 3- ylmethyl)cyclohexanecarboxamido) phenyl)acrylate | 371.3 |
| 5.2 | 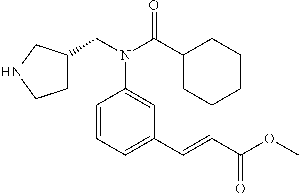 | (S,E)-Methyl 3-(3-(N-(pyrrolidin-3- ylmethyl)cyclohexanecarboxamido) phenyl)acrylate | 371.2 |

### Intermediate 6

### trans-4-(4-Chlorophenyl)cyclohexanecarbaldehyde

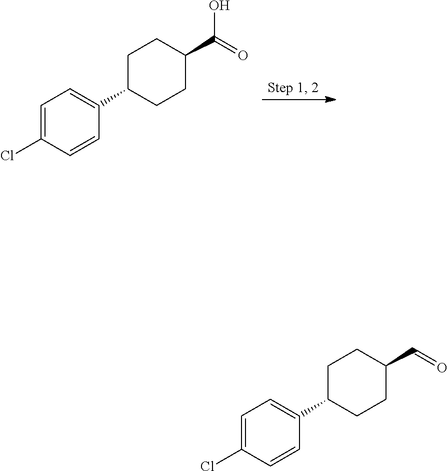 [SMILES: O=C(O)[C@H]1CC[C@H](c2ccc(Cl)cc2)CC1 ; O=C[C@H]1CC[C@H](c2ccc(Cl)cc2)CC1]

### Step 1: (trans-4-(4-Chlorophenyl)cyclohexyl)methanol

A solution of trans-4-(4-chlorophenyl)cyclohexanecarboxylic acid (2.0 g, 8.4 mmol) in THF (80 mL) was degassed with vacuum/nitrogen cycles (3×). Borane dimethyl sulfide complex (10 M, 1.7 mL, 17 mmol) was added dropwise to the reaction over 0.5 h at 25° C. The mixture stirred at 90° C. for 1.5 h and then cooled to 0° C. Methanol (20 mL) was added dropwise over 2 h. The reaction was concentrated and then diluted with DCM (60 mL) and water (30 mL). The organic phase was separated, dried over Na₂SO₄, filtered, and concentrated under reduced pressure to give (trans-4-(4-chlorophenyl)cyclohexyl)methanol (1.8 g, 81%) as a white solid. ¹H NMR (CDCl₃): δ 7.23 (d, 2H), 7.11 (d, 2H), 3.73 (t, 1H), 3.49 (d, 2H), 2.35-2.57 (m, 1H), 1.91 (d, 4H), 1.31-1.62 (m, 4H); MS: 207.1 [M−OH]⁺.

### Step 2: trans-4-(4-Chlorophenyl)cyclohexanecarbaldehyde

A mixture of (trans-4-(4-chlorophenyl)cyclohexyl)methanol (1.20 g, 5.34 mmol) and PCC (2.41 g, 6.4 mmol) in DCM (50 mL) was degassed with vacuum/nitrogen cycles (3×) and stirred at 25° C. for 2 h. Water (60 mL) was added to the reaction at 0° C., and the mixture was extracted with DCM (40 mL). The organic phase was washed with 1N HCl (2×30 mL), dried over anhydrous Na₂SO₄, filtered, and concentrated. The residue was purified by column chromatography (petroleum ether/ethyl acetate, 40:1) to give trans-4-(4-chlorophenyl)cyclohexanecarbaldehyde (700 mg, 50%) as a colorless oil. ¹H NMR (CDCl₃): δ 9.70 (s, 1H), 7.29-7.33 (m, 2H), 7.16 (d, J=8.53 Hz, 2H), 2.50 (d, 1H), 2.24-2.40 (m, 1H), 2.16 (d, 2H), 1.99-2.09 (m, 2H), 1.48-1.59 (m, 4H), 1.48 (d, 2H).

### Intermediate 7

### (E)-Methyl 3-(3-aminophenyl)acrylate

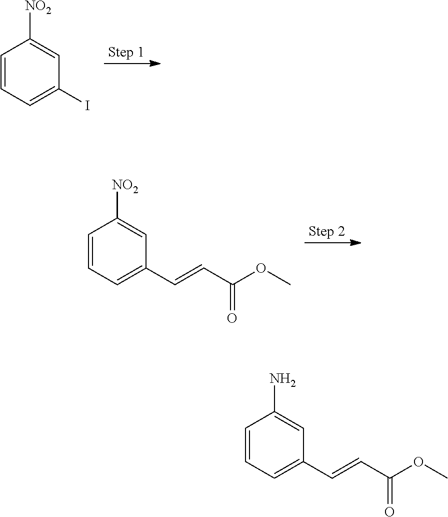

### Step 1: (E)-Methyl 3-(3-nitrophenyl)acrylate

A mixture of 1-iodo-3-nitro-benzene (30 g, 120 mmol), methyl prop-2-enoate (43 mL, 482 mmol), Pd₂(dba)₃ (5.52 g, 6.02 mmol), P(o-tolyl)₃ (9.17 g, 30.1 mmol) and TEA (83.5 mL, 602 mmol) in DMF (300 mL) was degassed with vacuum/nitrogen cycles (3×) and stirred at 90° C. for 12 h. The reaction mixture was filtered, diluted with water (800 mL), and extracted with ethyl acetate (3×400 mL). The combined organic layers were washed with water (3×800 mL), dried over anhydrous Na₂SO₄, filtered, and concentrated. Methyl tert-butyl ether (50 mL) was added to the residue, and the mixture was stirred for 2 h. The resulting solid was filtered and dried in vacuo to give methyl (E)-methyl 3-(3-nitrophenyl)acrylate (20 g, 74%) as a light yellow solid. ¹H NMR (CDCl₃): δ 8.39 (s, 1H), 8.25 (dd, 1H), 7.84 (d, 1H), 7.74 (d, 1H), 7.61 (t, 1H), 6.58 (d, 1H), 3.85 (s, 3H); MS: 208.0 [M±H]⁺.

### Step 2: (E)-Methyl 3-(3-aminophenyl)acrylate

A suspension of (E)-methyl 3-(3-nitrophenyl)acrylate (1.95 g, 9.4 mmol), tin (II) chloride dihydrate (7.20 g, 31.9 mmol), and methanol (27 mL) was heated at 65° C. for 2 h, cooled to room temperature, and then poured into ice water (100 mL). The reaction mixture was diluted with saturated NaHCO₃ (200 mL) and extracted with ethyl acetate (3×400 mL). The organic extracts were washed (200 mL brine), dried (Na₂SO₄), and concentrated under reduced pressure to give (E)-methyl 3-(3-aminophenyl)acrylate as yellow solid. ¹H NMR (400 MHz, DMSO-d₆): δ 7.48 (d, J=15.8 Hz, 1H), 7.06 (t, J=7.8 Hz, 1H), 6.85-6.79 (m, 2H), 6.65-6.61 (m, 1H), 6.41 (d, J=15.8 Hz, 1H), 5.19 (s, 2H), 3.71 (s, 3H).

### Intermediate 8

### trans-4-((tert-Butyldimethyl)silyl)oxy)cyclohexanecarbonyl chloride

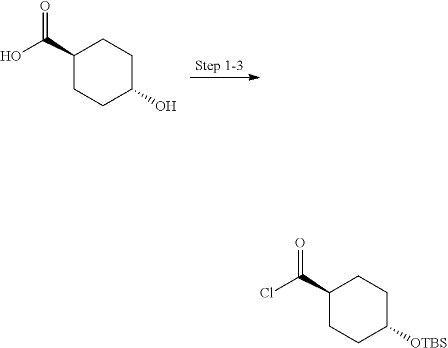 [SMILES: O=C(O)[C@H]1CC[C@H](O)CC1 ; CC(C)(C)[Si](C)(C)O[C@H]1CC[C@H](C(=O)Cl)CC1]

### Step 1: trans-tert-Butyldimethylsilyl 4-((tert-butyldimethylsilyl)oxy)cyclohexanecarboxylate

tert-Butyldimethylsilyl chloride (31.47 g, 208.8 mmol) was added to a mixture of trans-4-hydroxy-cyclohexanecarboxylic acid (10.03 g, 69.57 mmol), imidazole (18.96 g, 278.5 mmol), and DMF (140 mL) at rt under N₂ (reaction exothermed to 32° C.). The reaction was stirred at rt for 2 hours and then diluted with 300 mL diethyl ether. The organic layer was washed with 1 N HCl (2×300 mL), washed with 300 mL brine, dried (Na₂SO₄), filtered and concentrated to give trans-tert-butyldimethylsilyl 4-((tert-butyldimethylsilyl)oxy)cyclohexanecarboxylate (31.5 g) as a clear oil. ¹H NMR (400 MHz, DMSO-d₆): δ 3.61-3.53 (m, 1H), 2.26-2.18 (m, 1H), 2.04-1.96 (m, 2H), 1.92-1.85 (m, 2H), 1.51-1.39 (m, 2H), 1.39-1.27 (m, 2H), 0.94 (s, 9H), 0.89 (s, 9H), 0.26 (s, 6H), 0.06 (s, 6H).

### Step 2: trans-4-((tert-Butyldimethylsilyl)oxy)cyclohexanecarboxylic acid

Potassium carbonate (58.01 g, 419.7 mmol) in water (300 mL) was added to a mixture of trans-tert-butyldimethylsilyl 4-((tert-butyldimethylsilyl)oxy)cyclohexanecarboxylate (31.5 g crude, 69.6 mmol), ethanol (1000 mL) and THF (300 mL) at rt under N₂. The reaction was stirred at rt for 3 hours, concentrated until 300 mL remained, diluted with 600 mL brine, and then acidified to pH 2-3 with 20% NaHSO₄ (550 mL). The aqueous layer was extracted with 800 mL diethyl ether. The organic layer was washed with 800 mL brine, dried (Na₂SO₄), filtered and concentrated to give trans-4-((tert-butyldimethylsilyl)oxy)cyclohexanecarboxylic acid (17.3 g, 96% over 2 steps) as a white solid. ¹H NMR (400 MHz, DMSO-d₆): δ 12.30 (bs, 1H), 3.59-3.51 (m, 1H), 2.15-2.05 (m, 1H), 1.88-1.74 (m, 4H), 1.41-1.29 (m, 2H), 1.28-1.16 (m, 2H), 0.84 (s, 9H), 0.02 (s, 6H).

### Step 3: trans-4-((tert-Butyldimethylsilyl)oxy)cyclohexanecarbonyl chloride

Potassium carbonate (17.69 g, 128 mmol) in a round bottom flask under high vacuum was heated via heat gun for 5 min and then allowed to cool to rt. (Chloromethylene)dimethyl iminium chloride (6.47 g, 50.5 mmol) was added, the reaction was placed under N₂, and then toluene (100 mL) was added. After stirring for 10 min, trans-4-((tert-butyldimethylsilyl)oxy)cyclohexanecarboxylic acid (6.51 g, 25.2 mmol) was added. The reaction was stirred for 30 min, Celite was added to the reaction, and then the reaction was filtered through Celite with toluene washing (3×25 mL). The filtrate was partially concentrated to give a solution of trans-4-((tert-butyldimethylsilyl)oxy)cyclohexanecarbonyl chloride in toluene (18.55 g, 32.8 wt %). This solution was used immediately. ¹H NMR (400 MHz, DMSO-d₆): δ 3.66-3.58 (m, 1H), 2.76-2.67 (m, 1H), 2.23-2.14 (m, 2H), 1.98-1.89 (m, 2H), 1.67-1.56 (m, 2H), 1.44-1.32 (m, 2H), 0.91 (s, 9H), 0.08 (s, 6H).

### Intermediate 9

### trans-4-(4-Methoxy-3-methylphenyl)cyclohexanecarbaldehyde

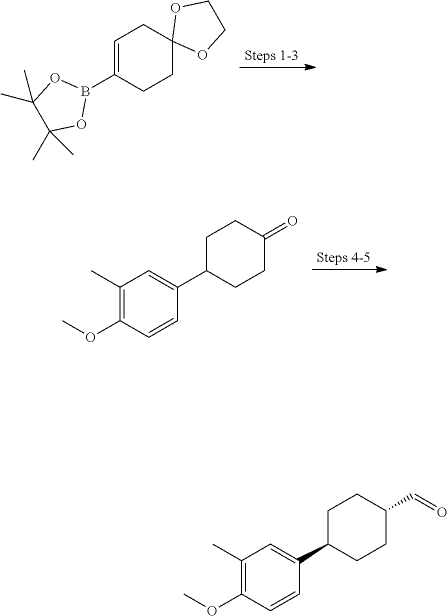 [SMILES: CC1(C)OB(C2=CCC3(CC2)OCCO3)OC1(C)C ; COc1ccc(C2CCC(=O)CC2)cc1C ; COc1ccc([C@H]2CC[C@H](C=O)CC2)cc1C]

### Step 1: 8-(4-methoxy-3-methylphenyl)-1,4-dioxaspiro[4.5]dec-7-ene

A mixture of 1,4-dioxa-spiro[4,5]dec-7-en-8-boronic acid pinacol ester (25.0 g, 93.9 mmol), 4-iodo-2-methylanisole (28.0 g, 113 mmol), 1,1′-bis(diphenylphosphino)ferrocene dichlorlopalladium(II) (1.38 g, 1.89 mmol), dioxane (470 mL) and 1M Na₂CO₃ (282 mL, 282 mmol) was degassed with vacuum/nitrogen cycles (3×), stirred at 50° C. for 2.5 h, and then allowed to cool to rt. The mixture was diluted with ethyl acetate (500 mL) and washed with saturated NaHCO₃ (500 mL×2). The aqueous layers were back extracted with ethyl acetate (200 mL). The combined ethyl acetate extracts were dried (Na₂SO₄), filtered, concentrated and purified by silica gel chromatography (0-5% ethyl acetate in hexanes) to give 8-(4-methoxy-3-methylphenyl)-1,4-dioxaspiro[4.5]dec-7-ene (19.9 g, 81%). ¹H NMR (400 MHz, DMSO-d₆): δ 7.21-7.16 (m, 2H), 6.85 (d, 1H), 5.89-5.84 (m, 1H), 3.90 (s, 4H), 3.76 (s, 3H), 2.52-2.47 (m, 2H), 2.32 (br s, 2H), 2.13 (s, 3H), 1.77 (t, 2H); MS: 261.1 [M+H]⁺.

### Step 2: 8-(4-Methoxy-3-methylphenyl)-1,4-dioxaspiro[4.5]decane

Palladium on carbon (10 wt %, 8.08 g, 7.59 mmol) was added to a solution of 8-(4-methoxy-3-methylphenyl)-1,4-dioxaspiro[4.5]dec-7-ene (19.8 g, 76.1 mmol) in ethyl acetate (300 mL) at rt under N₂. The N₂ inlet was replaced with a balloon of H₂. The reaction was stirred for 4.5 h, filtered through Celite with ethyl acetate, and then concentrated to give 8-(4-methoxy-3-methylphenyl)-1,4-dioxaspiro[4.5]decane (18.2 g; contains 13% ketone) as a white solid. ¹H NMR (400 MHz, DMSO-d₆): δ 7.00-6.95 (m, 2H), 6.81 (d, 1H), 3.91-3.84 (m, 4H), 3.73 (s, 3H), 2.49-2.42 (m, 1H), 2.11 (s, 3H), 1.76-1.68 (m, 4H), 1.67-1.55 (m, 4H); MS: 263.1 [M+H]⁺.

### Step 3: 4-(4-Methoxy-3-methylphenyl)cyclohexanone

Formic acid (96%, 14 mL, 356 mmol) and then water (2.20 mL, 122 mmol) were added to a solution of 8-(4-methoxy-3-methylphenyl)-1,4-dioxaspiro[4.5]decane (18.2 g) in toluene (60 mL) at rt under N₂. The reaction was heated at 120° C. for 4 hours, allowed to cool to rt, and then poured into 200 mL H₂O and 200 mL toluene. The toluene layer was washed with 200 mL H₂O and then 200 mL saturated NaHCO₃. The aqueous layers were back extracted with 100 mL toluene. The combined toluene extracts were dried (Na₂SO₄), filtered and concentrated to give 4-(4-methoxy-3-methylphenyl)cyclohexanone (15.5 g, 88% over 2 steps) as a white solid. ¹H NMR (400 MHz, DMSO-d₆): δ 7.08-7.03 (m, 2H), 6.84 (d, 1H), 3.74 (s, 3H), 3.00-2.91 (m, 1H), 2.61-2.51 (m, 2H), 2.28-2.20 (m, 2H), 2.12 (s, 3H), 2.06-1.98 (m, 2H), 1.88-1.76 (m, 2H); MS: 219.0 [M+H]⁺.

### Step 4: 1-Methoxy-4-(4-(methoxymethylene)cyclohexyl)-2-methylbenzene

A mixture of (methoxymethyl)triphenyl phosphonium chloride (35.74 g, 104.3 mmol) and THF (260 mL) under N₂ was cooled to −2.2° C. in an ice/brine bath. Sodium bis(trimethylsilyl)amide solution (2M in THF, 50 mL, 100 mmol) was added dropwise via addition funnel over 12 min (internal temp≤0.6° C.) with THF rinsing (5 mL). The reaction was stirred for 30 min, and then 4-(4-methoxy-3-methylphenyl)cyclohexanone (14.5 g, 66.6 mmol) was added portionwise over 5 min (exotherm to 7.3° C.). Residual cyclohexanone was rinsed into the reaction with THF (20 mL). The reaction was stirred at 0° C. for 25 min, and then poured into 400 mL H₂O and 400 mL toluene. The toluene layer was washed with 400 mL H₂O, dried (Na₂SO₄), filtered, concentrated and purified by silica gel chromatography (0-5% ethyl acetate in hexanes) to give 1-methoxy-4-(4-(methoxymethylene)cylcohexyl)-2-methylbenzene (15.6 g, 95%) as a pale gold oil. ¹H NMR (400 MHz, DMSO-d₆): δ 6.99-6.94 (m, 2H), 6.80 (d, 1H), 5.87 (s, 1H), 3.73 (s, 3H), 3.48 (s, 3H), 2.78-2.71 (m, 1H), 2.56-2.44 (m, 1H), 2.10 (s, 3H), 2.17-2.09 (m, 1H), 2.01-1.91 (m, 1H), 1.83-1.73 (m, 2H), 1.72-1.63 (m, 1H), 1.38-1.23 (m, 2H); MS: 247.1 [M+H]⁺.

### Step 5: trans-4-(4-Methoxy-3-methylphenyl)cyclohexanecarbaldehyde

Formic acid (96%, 12.5 mL, 331 mmol) and then water (2.5 mL, 139 mmol) were added to a solution of 1-methoxy-4-(4-(methoxymethylene)cylcohexyl)-2-methylbenzene (16.05 g, 65.15 mmol) in toluene (130 mL) under N₂. The reaction was heated at 120° C. for 2 hours, allowed to cool to rt, and then poured into 350 mL ethyl acetate and 350 mL H₂O. The organic layer was washed with 350 mL H₂O, dried (Na₂SO₄), filtered and concentrated to give 4-(4-methoxy-3-methylphenyl)cyclohexanecarbaldehyde (15.05 g) as a 1:1 mixture of stereoisomers. Aqueous sodium hydroxide (3.2 M, 31 mL, 99 mmol) was added to a solution of this mixture (14.68 g, 63.19 mmoL), toluene (60 mL) and ethanol (250 mL) at rt. The reaction was stirred for 5.5 hours (equilibration monitored by NMR) and then poured into 350 mL H₂O and 350 mL ethyl acetate. The organic layer was washed with 350 mL H₂O, and the aqueous layers were back extracted with 150 mL ethyl acetate. The combined extracts were dried (Na₂SO₄), filtered, concentrated and purified by silica gel chromatography (0-5% ethyl acetate in hexanes) to give trans-4-(4-methoxy-3-methylphenyl)cyclohexanecarbaldehyde (10.17 g, 69%) as a white solid. ¹H NMR (400 MHz, DMSO-d₆): δ 9.60 (s, 1H), 7.01-6.97 (m, 2H), 6.82 (d, 1H), 3.74 (s, 3H), 2.41-2.27 (m, 2H), 2.12 (s, 3H), 2.03-1.96 (m, 2H), 1.87-1.80 (m, 2H), 1.51-1.39 (m, 2H), 1.35-1.23 (m, 2H); MS: 233.0 [M+H]⁺.

### Intermediate 10

### trans-(E)-Methyl 3-(3-(((4-(4-methoxy-3-methylphenyl)cyclohexyl)methyl)amino)phenyl)acrylate

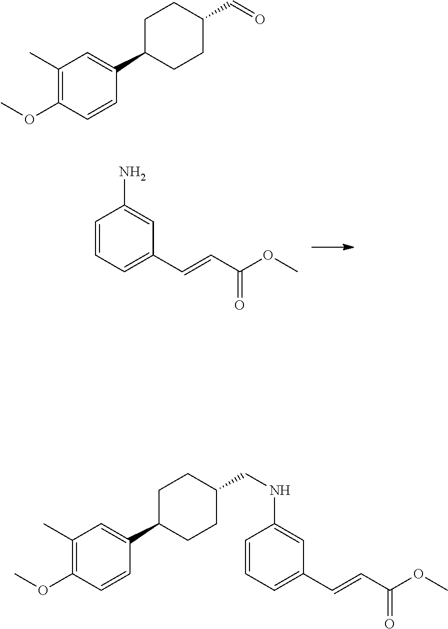

A solution of Intermediate 9 (2.78 g, 12.0 mmol), Intermediate 2.1 (2.34 g, 13.2 mmol), dichloroethane (60 mL), and acetic acid (1.35 mL, 23.6 mmol) under N₂ was cooled in an ice/water bath. Sodium triacetoxyborohydride (4.08 g, 19.3 mmol) was added in one portion, and the ice bath was removed. The reaction was stirred for 3 hours at rt, cooled in an ice/water bath, and then diluted with saturated NaHCO₃ (100 mL). The mixture was extracted with ethyl acetate (100 mL×2). Each extract was washed with saturated NaHCO₃ (100 mL). The combined extracts were washed with brine (100 mL). Some aqueous layers still contained product, so they were back extracted with ethyl acetate (50 mL). All of the extracts were combined, dried (Na₂SO₄), filtered, concentrated and purified by silica gel chromatography (0-20% ethyl acetate in hexanes) to give trans-(E)-methyl 3-(3-(((4-(4-methoxy-3-methylphenyl)cyclohexyl)methyl)amino)phenyl)acrylate (4.40 g, 93%) as a yellow solid. ¹H NMR (400 MHz, DMSO-d₆): δ 7.54 (d, 1H), 7.08 (t, 1H), 7.00-6.96 (m, 2H), 6.86-6.78 (m, 3H), 6.66 (d, 1H), 6.50 (d, 1H), 5.85-5.79 (m, 1H), 3.72 (d, 6H), 2.96-2.91 (m, 2H), 2.42-2.33 (m, 1H), 2.11 (s, 3H), 1.93 (d, 2H), 1.78 (d, 2H), 1.65-1.53 (m, 1H), 1.45-1.32 (m, 2H), 1.15-1.03 (m, 2H); LCMS: 394.7 [M+H]⁺.

The Intermediate below was synthesized following the procedures described for Intermediate 10.

| Int | Structure | Name | LCMS |
|---|---|---|---|
| 10.1 | 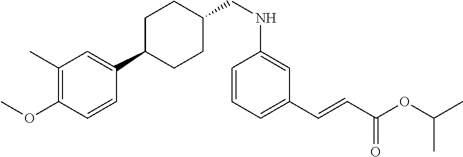 [SMILES: COc1ccc([C@H]2CC[C@H](CNc3cccc(/C=C/C(=O)OC(C)C)c3)CC2)cc1C] | trans-(E)-Isopropyl 3-(3-(((4-(4- methoxy-3- methylphenyl)cyclohexyl)methyl) amino)phenyl)acrylate | 422.7 |

### Compound 1

### (E)-Methyl 3-(3-(N-((6-(4-(dimethylamino)phenyl)pyridin-3-yl)methyl)cyclohexanecarboxamido)phenyl)acrylate

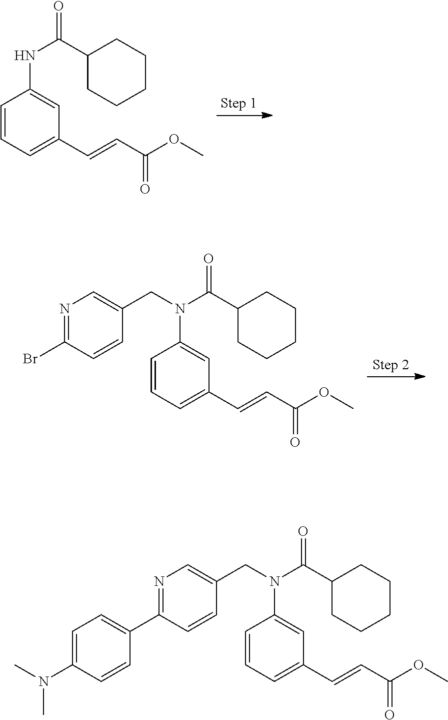

### Step 1: (E)-Methyl 3-(3-(N-((6-bromopyridin-3-yl)methyl)cyclohexanecarboxamido)phenyl) acrylate

Sodium hydride (134 mg, 3.34 mmol, 60% purity) was added to a solution of Intermediate 2 (800 mg, 2.78 mmol) in anhydrous THF (40 mL) at 0° C. After stirring the mixture for 0.5 h, 2-bromo-5-(bromomethyl)pyridine (837 mg) in anhydrous THF (2 mL) was added dropwise. The mixture was then stirred at 15° C. for 11.5 h, quenched with water (100 mL), and extracted with ethyl acetate (3×50 mL). The combined organic layers were washed with brine (100 mL), dried over anhydrous Na₂SO₄, filtered, and concentrated to give (E)-methyl 3-(3-(N-((6-bromopyridin-3-yl)methyl) cyclohexanecarboxamido)phenyl)acrylate (1.17 g, crude) as a light yellow oil.

### Step 2: (E)-Methyl 3-(3-(N-((6-(4-(dimethylamino)phenyl)pyridin-3-yl)methyl)cyclohexane carboxamido)phenyl)acrylate

A mixture of (E)-methyl 3-(3-(N-((6-bromopyridin-3-yl)methyl)cyclohexanecarboxamido)phenyl)acrylate (600 mg, crude), (4-(dimethylamino)phenyl)boronic acid (432 mg, 2.62 mmol), Cs₂CO₃ (640 mg, 1.96 mmol), and Pd(PPh₃)₄ (151 mg, 0.13 mmol) in DMF (3 mL) was degassed with vacuum/nitrogen cycles (3×) and then stirred at 90° C. for 12 h. The reaction was filtered, diluted with water (40 mL), and then extracted with ethyl acetate (3×30 mL). The combined organic layers were washed with brine (50 mL), dried over anhydrous Na₂SO₄, filtered, and concentrated. The residue was purified by reverse-phase HPLC to give (E)-methyl 3-(3-(N-((6-(4-(dimethylamino)phenyl)pyridin-3-yl)methyl)cyclohexanecarboxamido)phenyl)acrylate (299 mg, 41%, HCl salt) as a yellow solid. ¹H NMR (DMSO-d₆): δ 8.38 (s, 1H), 8.09 (d, 1H), 7.99 (d, 3H), 7.70-7.77 (m, 2H), 7.66 (d, 1H), 7.46 (t, 1H), 7.25 (d, 1H), 6.86 (d, 2H), 6.72 (d, 1H), 4.94 (br s, 2H), 3.72 (s, 3H), 3.02 (s, 6H), 2.17 (br s, 1H), 1.57-1.72 (m, 4H), 1.50 (d, 1H), 1.33-1.44 (m, 2H), 1.04-1.16 (m, 1H), 0.89 (d, 2H); MS: 498.3 [M+H]⁺.

### Compound 2

### (E)-Methyl 3-(3-(N-((1-(4-(dimethylamino)phenyl)piperidin-4-yl)methyl)cyclohexanecarboxamido)phenyl)acrylate

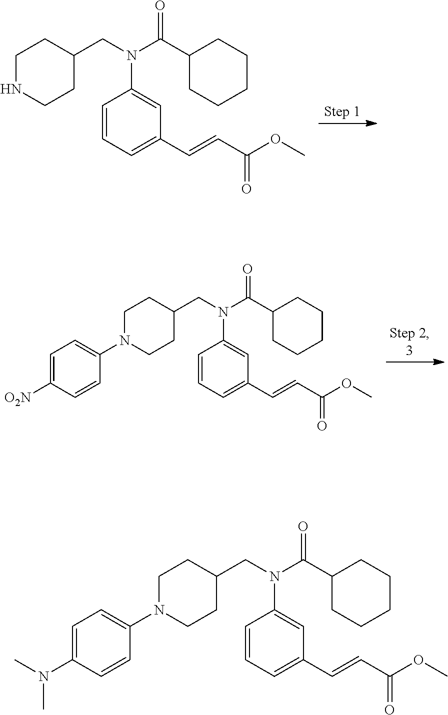

### Step 1: (E)-Methyl 3-(3-(N-((1-(4-nitrophenyl)piperidin-4-yl)methyl)cyclohexanecarboxamido)phenyl)acrylate

A mixture of Intermediate 5 (6.0 g, 15.6 mmol), 1-fluoro-4-nitrobenzene (1.8 mL, 17 mmol), and K₂CO₃ (4.31 g, 31.2 mmol) in DMF (20 mL) was degassed with vacuum/nitrogen cycles (3×) and stirred at 80° C. for 12 h. Water (100 mL) was added to the reaction, and the mixture was extracted with ethyl acetate (2×150 mL). The combined organic layers were washed with brine (300 mL), dried over Na₂SO₄, filtered and concentrated under reduced pressure. The residue was recrystallized from ethyl acetate (20 mL) to give (E)-methyl 3-(3-(N-((1-(4-nitrophenyl)piperidin-4-yl)methyl)cyclohexanecarboxamido)phenyl)acrylate (5.0 g, 53%) as a yellow solid. MS: 506.3 [M+H]⁺.

### Step 2: (E)-Methyl 3-(3-(N-((1-(4-aminophenyl)piperidin-4-yl)methyl)cyclohexanecarboxamido)phenyl)acrylate

A mixture of (E)-methyl 3-(3-(N-((1-(4-nitrophenyl)piperidin-4-yl)methyl)cyclohexanecarboxamido)phenyl)acrylate, Fe (2.76 g, 49.4 mmol), NH₄Cl (635 mg, 11.9 mmol), ethanol (50 mL), and water (50 mL) was stirred at 105° C. for 12 h, and then filtered and extracted with ethyl acetate (2×150 mL). The combined organic layers were washed with brine (300 mL), dried over Na₂SO₄, filtered, and concentrated under reduced pressure. The residue was purified by column chromatography (petroleum ether/ethyl acetate=30/1 to 1/1) to give (E)-methyl 3-(3-(N-((1-(4-aminophenyl)piperidin-4-yl)methyl)cyclohexanecarboxamido)phenyl)acrylate (3.4 g, 57%) as a black brown solid. MS: 476.4 [M+H]⁺.

### Step 3: (E)-Methyl 3-(3-(N-((1-(4-(dimethylamino)phenyl)piperidin-4-yl)methyl)cyclohexane carboxamido)phenyl)acrylate

A mixture of (E)-methyl 3-(3-(N-((1-(4-aminophenyl)piperidin-4-yl)methyl)cyclohexanecarboxamido)phenyl)acrylate (540 mg, 1.14 mmol), formaldehyde (952 mg, 11.4 mmol, 37% aqueous), and acetic acid (33 uL, 0.57 mmol) in methanol (10 mL) was stirred for 3 h. Sodium cyanoborohydride (215 mg, 3.42 mmol) was added, and the solution was stirred for another 12 h at 25° C. Water (100 mL) was added to the reaction, and the mixture was extracted with ethyl acetate (2×50 mL). The combined organic layers were washed with brine (200 mL), dried over Na₂SO₄, filtered, and concentrated under reduced pressure. The residue was purified by reverse-phase HPLC to give (E)-methyl 3-(3-(N-((1-(4-(dimethylamino)phenyl)piperidin-4-yl)methyl)cyclohexanecarboxamido)phenyl) acrylate (120 mg, 21%) as a yellow solid. ¹H NMR (400 MHz, DMSO-d₆) δ 7.64-7.79 (m, 3H), 7.51 (t, 1H), 7.36 (d, 1H), 6.69-6.83 (m, 3H), 6.64 (d, 2H), 3.73 (s, 3H), 3.57 (d, 2H), 3.40 (br s, 2H), 2.76 (s, 6H), 2.36-2.47 (m, 2H), 2.07 (br s, 1H), 1.54-1.73 (m, 6H), 1.17-1.52 (m, 6H), 0.99-1.16 (m, 1H), 0.73-0.97 (m, 2H). MS: 504.4 [M+I-1]⁺.

The Compounds below were synthesized following the procedures described for Compound 2

| Comp | Structure | Name | [M + H]⁺ |
|---|---|---|---|
| 3 | 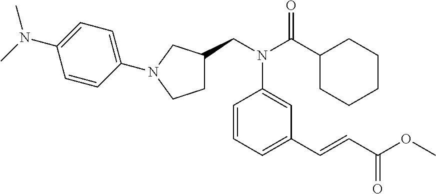 | (R,E)-Methyl 3-(3-(N-((1-(4- (dimethylamino)phenyl)pyrrolidin- 3- yl)methyl)cyclohexanecarboxamido) phenyl)acrylate | 490.4 |
| 4 | 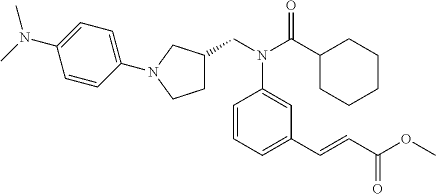 | (S,E)-Methyl 3-(3-(N-((1-(4- (dimethylamino)phenyl)pyrrolidin- 3- yl)methyl)cyclohexanecarboxamido) phenyl)acrylate | 490.4 |
| 4.1 | 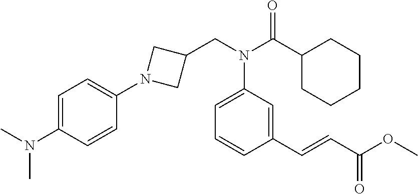 | (E)-Methyl 3-(3-(N-((1-(4- (dimethylamino)phenyl)azetidin-3- yl)methyl)cyclohexanecarboxamido) phenyl)acrylate | 476.5 |

### Compound 5

### (E)-Methyl 3-(3-(N-((1-(p-tolyl)piperidin-4-yl)methyl)cyclohexanecarboxamido)phenyl)acrylate

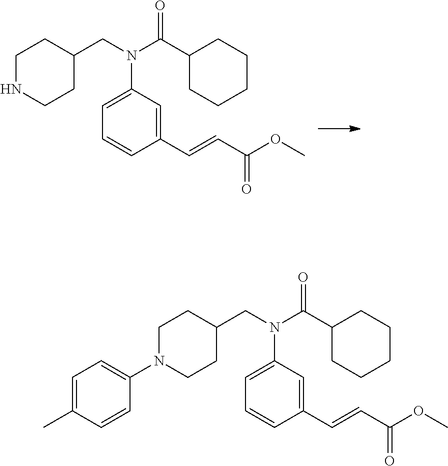

A mixture of Intermediate 5 (151 mg, 0.39 mmol), 4-bromotoluene (111 mg, 0.65 mmol), palladium acetate (10 mg, 0.043 mmol), BINAP (34 mg, 0.055 mmol), Cs₂CO₃ (260 mg, 0.80 mmol) and toluene (4 mL) was degassed with vacuum/nitrogen cycles (3×). The reaction was heated at 100° C. for 6.5 hours, diluted with ethyl acetate, and filtered through Celite. The filtrate was washed with water (2×20 mL), washed with brine (20 mL), dried (Na₂SO₄), filtered, concentrated, and purified by reverse-phase HPLC (37-55% acetonitrile/water w/0.1% TFA). The pure fractions were concentrated to dryness and then diluted with ethyl acetate (20 mL). This solution was washed with saturated NaHCO₃ (2×20 mL), washed with brine (20 mL), dried (Na₂SO₄), filtered and concentrated to give (E)-methyl 3-(3-(N-((1-(p-tolyl)piperidin-4-yl)methyl)cyclohexanecarboxamido)phenyl)acrylate (37 mg, 20%) as a pale yellow foam. ¹H NMR (400 MHz, DMSO-d₆): δ 7.79-7.68 (m, 3H), 7.52 (t, 1H), 7.36 (d, 1H), 6.98 (d, 2H), 6.81-6.73 (m, 3H), 3.72 (s, 3H), 3.60-3.51 (m, 4H), 2.55-2.45 (m, 2H), 2.17 (s, 3H), 2.11-2.02 (m, 1H), 1.70-1.45 (m, 8H), 1.40-1.15 (m, 4H), 1.15-1.02 (m, 1H), 0.92-0.80 (m, 2H); LCMS: 475.7 [M+H]⁺.

### Compound 6

### (E)-3-(3-(N-((1-(4-(Dimethylamino)phenyl)piperidin-4-yl)methyl)cyclohexanecarboxamido)phenyl)acrylic acid

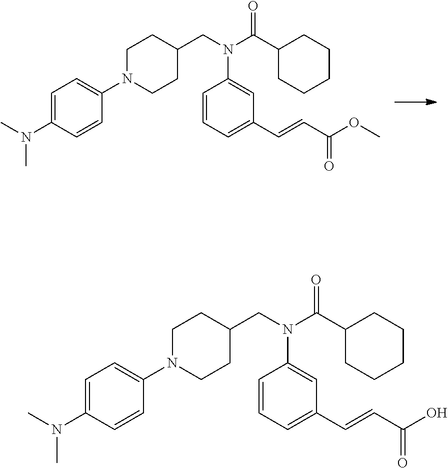

Aqueous sodium hydroxide (1N, 0.25 mL, 0.25 mmol) was added to a mixture of Compound 2 (25 mg, 0.050 mmol), THF (0.5 mL), and methanol (0.25 mL) at room temperature. The reaction mixture was stirred for 100 min, concentrated under reduced pressure, and diluted with water (5 mL) and ethyl acetate (20 mL). The mixture was acidified (1N HCl, pH=5), and the layers were separated. The organic layer was washed (20 mL brine), dried (Na₂SO₄), and concentrated under reduced pressure to give (E)-3-(3-(N-((1-(4-(dimethylamino)phenyl)piperidin-4-yl)methyl)cyclohexanecarboxamido)phenyl)acrylic acid (22 mg, 89%) as a blue grey solid. ¹H NMR (400 MHz, DMSO-d₆ w/ 1 drop of cone HCl): δ 7.83 (d, 2H), 7.75-7.70 (m, 2H), 7.62 (d, 1H), 7.55-7.48 (m, 1H), 7.44-7.36 (m, 3H), 6.68 (d, 1H), 3.68 (s, 2H), 3.50-3.41 (m, 4H), 3.01 (s, 6H), 2.11-2.00 (m, 1H), 1.90-1.80 (m, 5H), 1.66-1.52 (m, 4H), 1.50-1.42 (m, 1H), 1.38-1.26 (m, 2H), 1.10-1.00 (m, 1H), 0.90-0.76 (m, 2H); LCMS: 490.8 [M+H]⁺.

The Compound below was synthesized from Compound 8 following the procedure described for Compound 6.

| Comp | Structure | Name | [M + H]⁺ |
|---|---|---|---|
| 6.1 | 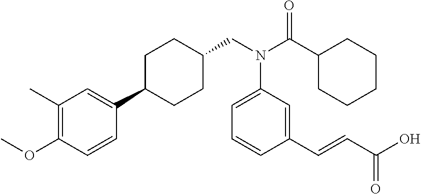 [SMILES: COc1ccc([C@H]2CC[C@H](CN(C(=O)C3CCCCC3)c3cccc(/C=C/C(=O)O)c3)CC2)cc1C] | trans-(E)-3-(3-(N-((4-(4-Methoxy- 3- methylphenyl)cyclohexyl)methyl) cyclohexanecarboxamido)phenyl) acrylic Acid | 490.4 |

### Compound 7

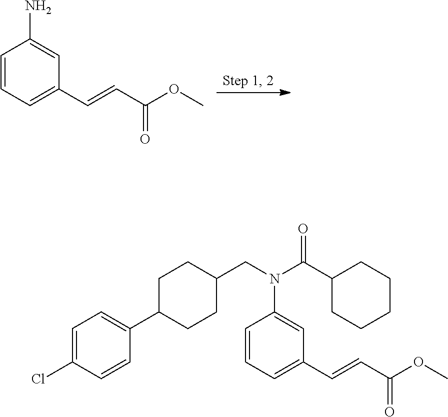 [SMILES: COC(=O)/C=C/c1cccc(N)c1 ; COC(=O)/C=C/c1cccc(N(CC2CCC(c3ccc(Cl)cc3)CC2)C(=O)C2CCCCC2)c1]

### Step 1: (E)-Methyl 3-(3-(((4-(4-chlorophenyl)cyclohexyl)methyl)amino)phenyl)acrylate

A mixture of Intermediate 6 (310 mg, 1.39 mmol), Intermediate 7 (271 mg, 1.53 mmol), acetic acid (79 uL, 1.4 mmol), and methanol (30 mL) was degassed with vacuum/nitrogen cycles (3×) and stirred at 25° C. for 1 h. Sodium cyanoborohydride (262 mg, 4.17 mmol) was added, and the mixture was stirred at 25° C. for 12 h. The reaction mixture was quenched with saturated NaHCO₃ (60 mL) at 0° C. and then extracted with ethyl acetate (60 ml). The organic phase was washed with brine (3×50 mL), dried over anhydrous Na₂SO₄, filtered and concentrated. The residue was purified by column chromatography (petroleum ether/ethyl acetate; 20:1) to give (E)-methyl 3-(3-(((4-(4-chlorophenyl)cyclohexyl)methyl)amino)phenyl)acrylate (533 mg, 85%) as a colorless oil. MS: 384.1 [M+H]⁺.

### Step 2: (E)-Methyl 3-(3-(N-((4-(4-chlorophenyl)cyclohexyl)methyl)-cyclohexanecarboxamido)phenyl)acrylate

Cyclohexanecarbonyl chloride (190 uL, 1.64 mmol) was added to a solution of (E)-methyl 3-(3-(((4-(4-chlorophenyl)cyclohexyl)methyl)amino)phenyl)acrylate (483 mg, 1.26 mmol) and TEA (350 uL, 2.52 mmol) in DCM (100 mL) over 2 h at 0° C. The mixture was heated to 25° C. for 3 h, quenched with saturated NaHCO₃ (30 mL), and then extracted with DCM (3×30 mL). The combined organic phases were washed with brine (2×30 mL), dried over anhydrous Na₂SO₄, filtered and concentrated. The residue was purified by reverse-phase HPLC to give (E)-methyl 3-(3-(N-((4-(4-chlorophenyl)cyclohexyl)methyl)cyclohexanecarboxamido)phenyl)acrylate (119 mg, 19%) as a light yellow solid. ¹H NMR (DMSO-d₆): δ 7.66-7.75 (m, 3H) 7.49 (t, 1H), 7.32 (d, 1H), 7.24-7.29 (m, 2H), 7.17-7.21 (m, 2H), 6.73 (d, 1H), 3.71 (s, 3H), 3.52 (d, 2H), 2.37-2.44 (m, 1H), 2.04 (br s, 1H), 1.73 (d, 4H), 1.57 (br s, 4H), 1.37-1.49 (m, 2H), 1.25-1.35 (m, 4H), 1.04 (t, 3H), 0.83 (d, 2H); MS: 494.2 [M+H]⁺.

### Compound 8

### trans-(E)-Methyl 3-(3-(N-((4-(4-methoxy-3-methylphenyl)cyclohexyl)methyl)cyclohexanecarboxamido)phenyl)acrylate

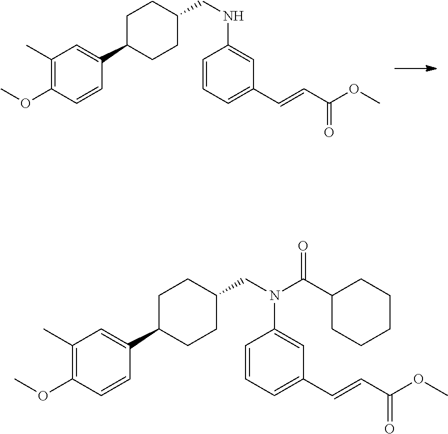

Cyclohexanecarbonyl chloride (2.2 mL, 16.4 mmol) was added to a solution of Intermediate 10 (3.26 g, 8.27 mmol), pyridine (2.7 mL, 33.3 mmol) and DCM (80 mL) at rt under N₂ (mild exotherm). The reaction was stirred for 15 minutes, poured into saturated NaHCO₃ (100 mL) and extracted with DCM (50 mL×2). The combined extracts were washed with brine. The brine layer was back extracted with DCM (50 mL). All of the extracts were combined, dried (Na₂SO₄), filtered, concentrated and purified by silica gel chromatography (0-20% ethyl acetate in hexanes) to give trans-(E)-methyl 3-(3-(N-((4-(4-methoxy-3-methylphenyl)cyclohexyl)methyl)cyclohexanecarboxamido)phenyl)acrylate (2.34 g, 57%) as a white foam. The mixed fractions were repurified to provide additional product. ¹H NMR (400 MHz, DMSO-d₆): δ 7.78-7.69 (m, 3H), 7.52 (t, 1H), 7.35 (d, 1H), 6.97-6.92 (m, 2H), 6.80-6.73 (m, 2H), 3.73 (dd, 6H), 3.57-3.52 (m, 2H), 2.37-2.28 (m, 1H), 2.09 (s, 3H), 2.08-2.03 (m, 1H), 1.78-1.68 (m, 4H), 1.65-1.53 (m, 4H), 1.52-1.44 (m, 1H), 1.40-1.23 (m, 5H), 1.11-0.98 (m, 3H), 0.92-0.79 (m, 2H); MS: 504.6 [M+H]⁺.

The Compounds below were synthesized following the procedures described for Compound 8.

| Comp | Structure | Name | [M + H]⁺ |
|---|---|---|---|
| 9 | 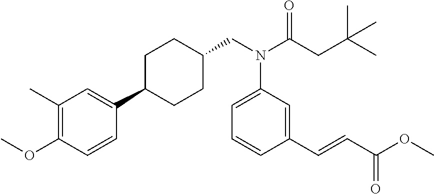 | trans-(E)-Methyl 3-(3-(N-((4-(4- methoxy-3- methylphenyl)cyclohexyl)methyl)- 3,3- dimethylbutanamido)phenyl)acrylate | 492.4 |
| 10 | 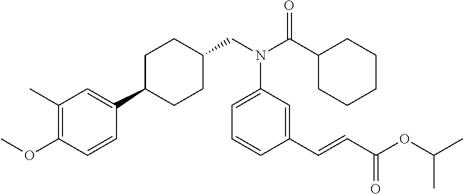 [SMILES: COc1ccc([C@H]2CC[C@H](CN(C(=O)C3CCCCC3)c3cccc(/C=C/C(=O)OC(C)C)c3)CC2)cc1C] | trans-(E)-Isopropyl 3-(3-(N-((4-(4- methoxy-3- methylphenyl)cyclohexyl)methyl) cyclohexanecarboxamido)phenyl) acrylate | 532.5 |
| 11 | 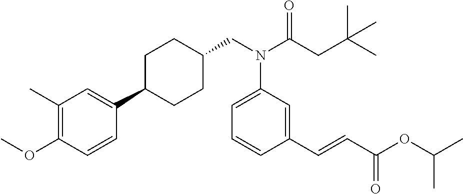 [SMILES: COc1ccc([C@H]2CC[C@H](CN(C(=O)CC(C)(C)C)c3cccc(/C=C/C(=O)OC(C)C)c3)CC2)cc1C] | trans-(E)-Isopropyl 3-(3-(N-((4-(4- methoxy-3- methylphenyl)cyclohexyl)methyl)- 3,3- dimethylbutanamido)phenyl)acrylate | 520.6 |
| 12 | 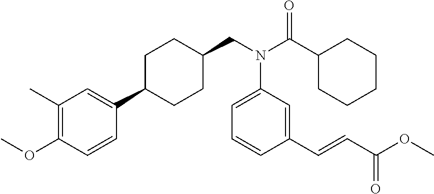 | cis-(E)-Methyl 3-(3-(N-((4-(4- methoxy-3- methylphenyl)cyclohexyl)methyl) cyclohexanecarboxamido)phenyl) acrylate | 504.4 |

### Compound 13

### trans-(E)-Isopropyl 3-(3-(trans-4-hydroxy-N-((4-(4-methoxy-3-methylphenyl)cyclohexyl)methyl)cyclohexanecarboxamido)phenyl)acrylate

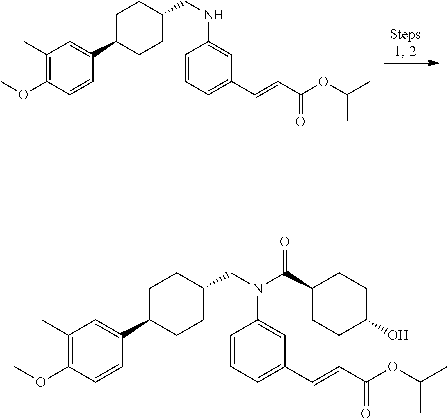

### Step 1: trans-(E)-Isopropyl 3-(3-(trans-4-((tert-butyldimethylsilyl)oxy)-N-((4-(4-methoxy-3-methylphenyl)cyclohexyl)methyl)cyclohexanecarboxamido)phenyl)acrylate

Intermediate 8 (2.5 mL, 9.03 mmol) was added dropwise to a yellow solution of Intermediate 10.1 (1.64 g, 3.90 mmol), pyridine (1.2 mL, 15 mmol) and dichloromethane (32 mL) at rt under N₂. The reaction was stirred for 10 minutes, poured into 50 mL saturated NaHCO₃ and extracted with ethyl acetate (50 mL×2). The combined organics were washed with 50 mL brine, dried (Na₂SO₄), filtered, concentrated and purified by silica gel chromatography (0-20% ethyl acetate in hexanes) to give trans-(E)-isopropyl 3-(3-(trans-4-((tert-butyldimethylsilypoxy)-N-((4-(4-methoxy-3-methylphenyl)cyclohexyl)methyl)cyclohexanecarboxamido)phenyl)acrylate (2.78 g) as a white foam. ¹H NMR (400 MHz, DMSO-d₆): δ 7.78-7.64 (m, 3H), 7.51 (t, 1H), 7.34 (d, 1H), 6.97-6.92 (m, 2H), 6.80-6.70 (m, 2H), 5.06-4.99 (m, 1H), 3.72 (s, 3H), 3.59-3.45 (m, 3H), 2.37-2.28 (m, 1H), 2.09 (s, 3H), 2.04-1.96 (m, 1H), 1.89-1.59 (m, 10H), 1.48-1.34 (m, 3H), 1.26 (d, 6H), 1.34-1.20 (m, 2H), 1.09-0.97 (m, 2H), 0.79 (s, 9H), −0.03 (s, 6H); MS: 662.6 [M+H]⁺.

### Step 2: trans-(E)-Isopropyl 3-(3-(trans-4-hydroxy-N-((4-(4-methoxy-3-methylphenyl)cyclohexyl)methyl)cyclohexanecarboxamido)phenyl)acrylate

Aqueous hydrochloric acid (6N, 5.5 mL, 33 mmol) was added dropwise to a solution of trans-(E)-isopropyl 3-(3-(trans-4-((tert-butyldimethylsilyl)oxy)-N-((4-(4-methoxy-3-methylphenyl)cyclohexyl)methyl)cyclohexanecarboxamido)phenyl)acrylate (2.58 g, 3.90 mmol), THF (13 mL) and methanol (13 mL) at rt. At the beginning of the addition, an exotherm was observed so the reaction was cooled in an ice/water bath before completing the addition. The bath was pulled when the addition was complete. The reaction was stirred at rt for 2 hours, cooled in ice/water bath, quenched with 50 mL saturated NaHCO₃, and then extracted with 50 mL ethyl acetate. The organic layer was washed with 50 mL saturated NaHCO₃, washed with 50 mL brine, dried (Na₂SO₄), filtered, concentrated and purified by silica gel chromatography (40-80% ethyl acetate in hexanes) to give trans-(E)-Isopropyl 3-(3-(trans-4-hydroxy-N-((4-(4-methoxy-3-methylphenyl)cyclohexyl)methyl)cyclohexanecarboxamido)phenyl)acrylate (1.50 g) as a white foam. The mixed fractions were repurified to provide additional product (358 mg, 87% over 2 steps). ¹H NMR (400 MHz, DMSO-d₆): δ 7.78-7.65 (m, 3H), 7.50 (t, 1H), 7.35 (d, 1H), 6.97-6.92 (m, 2H), 6.80-6.71 (m, 2H), 5.08-4.98 (m, 1H), 4.40 (br s, 1H), 3.71 (s, 3H), 3.59-3.50 (m, 2H), 3.30-3.21 (m, 1H), 2.32 (t, 1H), 2.09 (s, 3H), 2.01-1.92 (m, 1H), 1.78-1.67 (m, 6H), 1.62 (d, 2H), 1.45-1.32 (m, 3H), 1.26 (d, 6H), 1.32-1.21 (m, 2H), 1.10-0.97 (m, 2H), 0.80-0.67 (m, 2H); MS: 548.6 [M+H]⁺.

The Compound below was synthesized following the procedures described for Compound 13.

| Comp | Structure | Name | [M + H]⁺ |
|---|---|---|---|
| 14 | 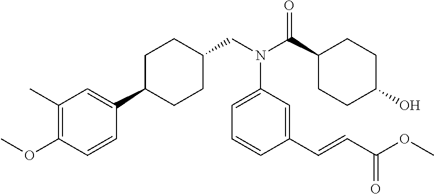 | trans-(E)-Methyl 3-(3-(trans-4- hydroxy-N-((4-(4-methoxy-3- methylphenyl)cyclohexyl)methyl) cyclohexanecarboxamido)phenyl) acrylate | 520.6 |

### Example A-1: Parenteral Pharmaceutical Composition

To prepare a parenteral pharmaceutical composition suitable for administration by injection (subcutaneous, intravenous), 1-1000 mg of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 10-500 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound described herein, or a pharmaceutically acceptable salt thereof, is placed into Size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl celluose, propylene glycol, isopropyl myristate and purified alcohol USP. The resulting gel mixture is then incorporated into containers, such as tubes, which are suitable for topical administration.

### Example B-1: In Vitro FXR Assay (PGL3)

### Seeding

CV-1 cells were seeded at a density of 2,000,000 cells in a T175 flask with DMEM+10% charcoal double-stripped FBS and incubated at 37° C. in a 5% CO₂ for 18 h (0/N).

### Transfection

After 18 h of incubation, the medium in the T175 flask was changed with fresh DMEM+10% charcoal super-stripped serum.

To prepare the transfection reaction mixture, the transfection reagent (X-tremeGENE HP from Roche, Cat #06 366 236 001) was added in to a 1.5 mL microcentrifuge tube labelled “A” at a ratio of 1:3 (DNA in μg: transfection reagent in μL). OptiMEM medium (Life Technologies, Cat #31985-062) was added accordingly to provide a total volume of 1 mL. The transfection reaction mixture was then briefly vortexed and incubated at room temperature for 5 minutes.

In a separate 1.5 mL microcentrifuge tube labelled “B”, 100 μL OptiMEM and plasmids XPD90Gal pCMXhFXRfl, pCMXhRXRfl+PGL3-ECRE*6-luc+CMX-YFP in the ratio 2 μg: 2 μg:18 μg:3 μg were added. This microcentrifuge tube “B” was then briefly vortexed and incubated at room temperature for 5 minutes.

The total volume in tube labelled “A” was then transferred in to tube labelled “B.” The mixture was then briefly vortexed and the transfection:DNA complex was then incubated for about 15-20 minutes at room temperature.

Following incubation, the transfection reagent/DNA mixture complex was then added to cells in the T175 flask and the cells were incubated at 8 h (0/N) at 37° C. in 5% CO₂.

### Test Compounds

In a 96 well plate, a half logarithmic serial dilution was prepared. OptiMEM was used as the diluent. Using any of one of the compounds described herein, a compound stock solution of 10 mM was prepared. An initial 1:100 dilution was made into the first well for a final concentration of 100 μM. The final concentrations in the 96-well plate was prepared by using a multichannel pipette to transfer 5 μL of the diluted compound to 384-well plate in quadruplicate.

Cells in T175 flask were trypsinized and cells were resuspending in 40 mL phenol red free DMEM+10% charcoal super-stripped FBS. Typically, one T175 flask was sufficient to seed two 384 well plates. CV-1 cells were seeded at 45 μL cell suspension/well using a multi-channel pipet or a 384 multidrop dispenser. The cells were then incubated for 18 hrs, overnight.

### Reading

After removing the plates from the incubator, the medium was flicked out of plate and 384 well plate was turned upside down onto paper towel. The remaining medium was gently tapped out. 15 μL lysis buffer was then added to each well using a multichannel pipet or a 384 well multi-drop dispenser. After incubation for 10 minutes at room temperature on shaker, 30 μL Luciferase buffer was added to each well. Luminescence counts were taken immediately using the Perkin Elmer Envision.

The ability of the compounds disclosed herein to inhibit FXR activity was quantified and the respective EC₅₀ value was determined. Table 3 provides activity of various compounds disclosed herein. Fex=fexaramine.

TABLE 3

| Compound No | Cmpd/Fex* |
|---|---|
| 1 | +++ |
| 2 | +++ |
| 3 | + |
| 4 | ++ |
| 5 | +++ |
| 6 | + |
| 7 | +++ |

*Where ‘Cmpd/Fex’ denotes the ratio of the EC₅₀ for the test compound relative to the EC₅₀ of the Fexaramine control.
‘+++’ means Cmpd/Fex <10;
‘++’ means Cmpd/Fex >10 & <100;
‘+’ means Cmpd/Fex >100.

### Example B-2: In Vitro FXR Assay (TK)

### Seeding

CV-1 cells were seeded at a density of 2,000,000 cells in a T175 flask with DMEM+10% charcoal double-stripped FBS and incubated at 37° C. in 5% CO₂ for 18 h (0/N).

### Transfection

After 18 h of incubation, the medium in the T175 flask was changed with fresh DMEM+10% charcoal super-stripped serum. In a polypropylene tube, 2500 μL OptiMEM (Life Technologies, Cat #31985-062) was combined with expression plasmids for hFXR, hRXR, TK-ECRE-luc and pCMX-YFP. The tube was then briefly vortexed and incubated at room temperature for 5 minutes. Transfection reagent (X-tremeGENE HP from Roche, Cat #06 366 236 001) was added to the OptiMEM/plasmid mixture vortexed and incubated at room temperature for 20 minutes. Following incubation, the transfection reagent/DNA mixture complex was added to cells in the T175 flask and the cells were incubated at 37° C. in 5% CO₂ for 18 h (O/N).

### Test Compounds

Compounds were serially diluted in DMSO and added to transfected CV-1 cells. The cells were then incubated for 18 hrs. The next day cells were lysed and examined for luminescence.

Representative data for exemplary compounds disclosed herein is presented in the following table.

TABLE 4

| Compound | TK hFXR: |
|---|---|
| No | EC50 (μM)** |
| 1 | ++ |
| 2 | +++ |
| 3 | + |
| 4 | ++ |
| 4.1 | + |
| 5 | +++ |
| 6 | + |
| 6.1 | ++ |
| 7 | +++ |
| 8 | +++ |
| 9 | +++ |
| 10 | +++ |
| 11 | +++ |
| 12 | + |
| 13 | +++ |
| 14 | +++ |

**Where ‘+++’ means Cmpd ≤1 uM;
‘++’ means Cmpd >1 & <10 uM;
‘+’ means Cmpd ≥10 uM.
Compounds with a maximum efficacy of <25% of the Fexarmine control were classified as ’+’

### Example B-3: NASH Activity Study (STZ Model)

NASH can be induced in male C57BL/6 by a single subcutaneous injection of 200 ug STZ 2 days after birth followed by feeding high fat diet (HFD) ad libitum after 4 weeks of age. While continuing HFD, compounds can be dosed for 4-8 weeks to determine the effects on NASH. Fasting glucose can be measured throughout the study with a hand held glucose meter. Serum alanine aminotransferase (ALT), aspartate aminotransferase (AST) and triglyceride (TG) can be measured by a clinical chemistry analyzer. The contents of TG in the liver tissue can be measured using the Triglyceride E-test kit (Wako, Tokyo, Japan). Histological analysis of liver sections can be performed on tissue embedded in Tissue-TEK O.C.T. compound, snap frozen in liquid nitrogen, and stored at −80 C. The sections can be cut (5 urn), air dried and fixed in acetone. For hematoxylin and eosin staining, liver sections can be prefixed by Bouin's solution and then stained with hematoxylin and eosin solution. The degree of (zone-3) liver fibrosis can be assessed with Siriusred staining.

### Example B-4 NASH Activity Study (AMEN Model)

NASH is induced in male C57BL/6 mice by diet-induction with AMLN diet (DIO-NASH) (D09100301, Research Diet, USA) (40% fat (18% trans-fat), 40% carbohydrates (20% fructose) and 2% cholesterol). The animals are kept on the diet for 29 weeks. After 26 weeks of diet induction, liver biopsies are performed for base line histological assessment of disease progression (hepatosteatosis and fibrosis), stratified and randomized into treatment groups according to liver fibrosis stage, steatosis score, and body weight. Three weeks after biopsy the mice are stratified into treatment groups and dosed daily by oral gavage with FXR agonists for 8 weeks. At the end of the study liver biopsies are performed to assess hepatic steatosis and fibrosis by examining tissue sections stained with H&E and Sirius Red, respectively. Total collagen content in the liver is measured by colorimetric determination of hydroxyproline residues by acid hydrolysis of collagen. Triglycerides and total cholesterol content in liver homogenates are measured in single determinations using autoanalyzer Cobas C-111 with commercial kit (Roche Diagnostics, Germany) according to manufacturer's instructions.

### Example B-5: Intrahepatic Cholestasis Model

Experimental intrahepatic cholestasis induced by 17a-ethynylestradiol (EE2) treatment in rodents is a widely used in vivo model to examine the mechanisms involved in estrogen-induced cholestasis. Intrahepatic cholestasis can be induced in adult male mice by subcutaneous injection of 10 mg/kg 17a-ethynylestradiol (E2) daily for 5 days. Testing of FXR ligands can be performed by administration of compounds during E2 induction of cholestasis. Cholestatic effects can be quantitated by assessing liver/body weight ratio and measuring serum total bile acids and alkaline phosphatase levels can be measured using reagents and controls from Diagnostic Chemicals Ltd. and the Cobas Mira plus CC analyzer (Roche Diagnostics). For histology and mitosis measurements, liver samples from each mouse can be fixed in 10% neutral buffered formalin. Slides are stained with hematoxylin and eosin using standard protocols and examined microscopically for structural changes. Hepatocyte proliferation is evaluated by immunohistochemical staining for Ki67.

### Example B-6: Direct Target Gene Regulation

Direct target gene regulation by FXR ligands can be assessed by dosing mice either acutely or chronically with compounds, and collecting tissues at various time points after dosing. RNA can be isolated from tissues such as the ileum and liver, and reverse transcribed to cDNA for quantitative PCR analysis of genes known in the literature to be directly and indirectly regulated by FXR such as SHP, BSEP, IBABP, FGF15, Cyp7a1, Cyp8b1 and C3.

### Example B-7: Mouse PK Study

The plasma pharmacokinetics of any one of the compounds disclosed herein as a test article test article is measured following a single bolus intravenous and oral administration to mice (CD-1, C57BL, and diet induced obesity mice). Test article is formulated for intravenous administration in a vehicle solution of DMSO, PEG400, hydroxypropyl-β-cyclodextrin (HPβCD) and is administered at a dose volume of 3 mL/kg at selected dose levels. An oral dosing formulation is prepared in appropriate oral dosing vehicles (vegetable oils, PEG400, Solutol, citrate buffer, or carboxymethyl cellulose) and is administered at a dose volume of 5-10 mL/kg at selected dose levels. Blood samples (approximately 0.15 mL) are collected by cheek pouch method at pre-determined time intervals post intravenous or oral doses into tubes containing EDTA. Plasma is isolated by centrifugation of blood at 10,000 g for 5 minutes, and aliquots are transferred into a 96-well plate and stored at −60° C. or below until analysis.

Calibration standards of test article are prepared by diluting DMSO stock solution with DMSO in a concentration range. Aliquots of calibration standards in DMSO are combined with plasma from naïve mouse so that the final concentrations of calibration standards in plasma are 10-fold lower than the calibration standards in DMSO. PK plasma samples are combined with blank DMSO to match the matrix. The calibration standards and PK samples are combined with ice-cold acetonitrile containing an analytical internal standard and centrifuged at 1850 g for 30 minutes at 4° C. The supernatant fractions are analyzed by LC/MS/MS and quantitated against the calibration curve. Pharmacokinetic parameters (area under the curve (AUC), Cₘₐₓ, Tₘₐₓ, elimination half-life (T_{1/2}), clearance (CL), steady state volume of distribution (V_{dss}), and mean residence time (MRT)) are calculated via non-compartmental analysis using Microsoft Excel (version 2013).

### Example B-8: Rat ANIT Model

A compound described herein is evaluated in a chronic treatment model of cholestasis over a range of doses from 0.01 to 10 mg/kg. This model is used to evaluate the suitability of the use of FXR agonists, e.g. a compound described herein, for the treatment of cholestatic liver disorders such as bile acid malabsorption (e.g., primary or secondary bile acid diarrhea), bile reflux gastritis, collagenous colitis, lymphocytic colitis, diversion colitis, indeterminate colitis, Alagille syndrome, biliary atresia, ductopenic liver transplant rejection, bone marrow or stem cell transplant associated graft versus host disease, cystic fibrosis liver disease, and parenteral nutrition-associated liver disease.

Rats are treated with alpha-naphthylisothiocyanate (ANIT) (0.1% w/w) in food for 3 days prior to treatment with a compound described herein at doses from 0.01 to 10 mg/kg (“Veh”). A noncholestatic control group is fed standard chow diet without ANIT, and serves as the noncholestatic control animals (“Control”). After 14 days of oral dosing, rat serum is analyzed for levels of analytes. LLQ, lower limit of quantitation. Mean±SEM; n=5.

Levels of hepatobiliary injury indicators are measured in rat serum, such as elevated levels of circulating aspartate aminotransferase (AST), alanine aminotransferase (ALT), bilirubin and bile acids. ANIT exposure induces profound cholestasis and hepatocellular damage. A compound that improves many of these indicators is useful in the treatment of the aforementioned diseases or conditions.

Reductions in the accumulation of bile acids in the liver, enhancements in bile acid excretion in the biliary tract and inhibition of bile acid synthesis is consistent with the pharmacological action of a FXR agonist. An improvement in the serum conjugated bilirubin (a direct indicator for hepatic function) implies recovery from cholestasis with improved bile excretion.

Furthermore, an analysis is made to ascertain the effects of the compound described herein on serum FGF15 fibroblast growth factor 15 (FGF15 in rodent; FGF19 in human) expression, a hormone that is secreted in the portal blood and signals to the liver to repress Cyp7a1 expression synergistically with SHP. The direct FXR-dependent induction of FGF15/19 along with FGF15/19's anti-cholestatic properties makes it a convenient serum biomarker for detecting target engagement of FXR agonists.

Serum FGF15 levels are quantified using an FGF15 Meso Scale Discovery (MSD) assay. For example, Mouse FGF15 antibody from R&D Systems (AF6755) is used both as capture and detection antibody in the assay. MSD SULFO-TAG NHS-Ester is used to label the FGF15 antibody. MSD standard 96-well plates are coated with the FGF15 capture antibody and the plates are blocked with MSD Blocker A (R93AA-2). After washing the plate with PBS+0.05% Tween 20, MSD diluent 4 is dispensed into each well and incubated for 30 min. 25 pi of calibrator dilutions or samples (serum or EDTA plasma) are dispensed into each well and incubated with shaking at RT.

After washing, detection antibody is added and incubated with shaking for 1 h at RT. After washing and the addition of MSD Read buffer (R92TC-2), the plate is read on an MSD SECTOR Imager 6000. Plots of the standard curve and unknown samples are calculated using MSD data analysis software.

The examples and embodiments described herein are for illustrative purposes only and various modifications or changes suggested to persons skilled in the art are to be included within the spirit and purview of this application and scope of the appended claims.

What is claimed is:

1. A compound that has the structure of Formula (VIII), or a pharmaceutically acceptable salt or solvate thereof: 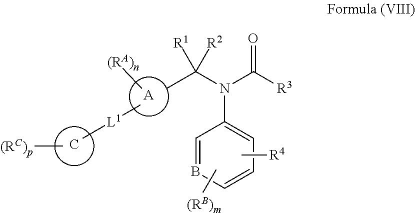
wherein
R¹ and R² are each independently selected from H, D, F, C₁-C₄alkyl, or C₁-C₄fluoroalkyl;
or R¹ and R² are taken together with the carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NR¹⁰C(═O)R¹¹, NR¹⁰C(O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
L⁴ is absent, —O—, —S—, —S(═O)—, —S(═O)₂, —NR¹⁰, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, (CH₂)ᵣ—, or —(OCH₂CH₂)ᵣ—, r is 1, 2, 3, or 4;
L⁵ is absent, unsubstituted or substituted C₁-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
R¹³ is H, halogen, —N(R¹⁰)₂, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
L³ is —C(R⁵)(R⁶)—, —C(R⁵)(R⁶)—C(R⁷)(R⁸)—, —O—C(R⁷)(R⁸)—, or —C(R⁵)(R⁶)—O—;
R⁵ and R⁷ are each independently selected from H, D, C₁-C₄alkyl and C₃-C₆cycloalkyl;
or R⁵ and R⁷ are taken together with the intervening atoms to form a double bond;
or R⁵ and R⁷ are taken together with the intervening atoms to form an epoxide or an substituted or unsubstituted C₃-C₆cycloalkyl;
R⁶ and R⁸ are each independently selected from H, D, C₁-C₄alkyl or C₃-C₆cycloalkyl;
Y is —CH₂OR⁹, —C(═O)OR⁹, 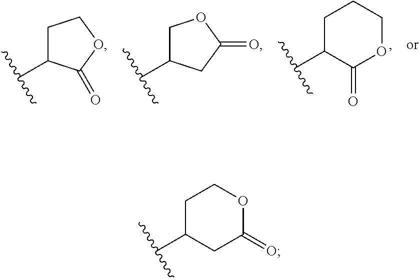 [SMILES: *C1CCOC1=O ; *C1COC(=O)C1 ; *C1CCCOC1=O ; *C1CCOC(=O)C1]
R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, or substituted or substituted or unsubstituted heterocycle;
L is —X¹-L²-, -L²-X¹—;
X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹⁰—, —NR¹⁰S(═O)₂—, or —NR¹⁰—;
L² is absent or substituted or unsubstituted C₁-C₄alkylene;
ring A is a C₃-C₁₀cycloalkyl;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
B is CR^{B}, or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

2. The compound of claim 1, or a pharmaceutically acceptable salt or solvate thereof, wherein:
ring A is C₃-C₁₀cycloalkyl that is selected from cyclopropyl, cyclobutyl, cyclopentyl, cyclohexyl, cycloheptyl, and cyclooctyl.

3. The compound of claim 1 or claim 2, or a pharmaceutically acceptable salt or solvate thereof, wherein:
ring A is

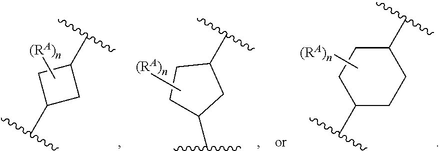

4. The compound of any one of claims 1-3, or a pharmaceutically acceptable salt or solvate thereof, wherein:
L is —X¹-L²-, -L²-X¹—;
X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, or —NR¹⁰—;
L² is absent or —CH₂—.

5. The compound of any one of claims 1-4, or a pharmaceutically acceptable salt or solvate thereof, wherein:
L¹ is absent, —O—, —S—, —S—CH₂—, —CH₂—S—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, —NR¹⁰—, —NR¹⁰—CH₂—, or —CH₂—NR¹⁰—.

6. The compound of any one of claims 1-5, or a pharmaceutically acceptable salt or solvate thereof, wherein:
R⁴ is

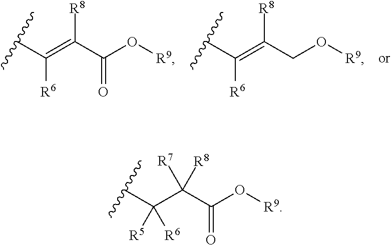

7. The compound of claim 6, or a pharmaceutically acceptable salt or solvate thereof, wherein:
R⁴ is

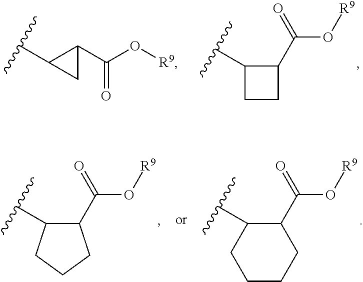

8. The compound of any one of claims 1-5, or a pharmaceutically acceptable salt or solvate thereof, wherein:
R⁴ is -L³-Y;
L³ is —CH₂—;
Y is

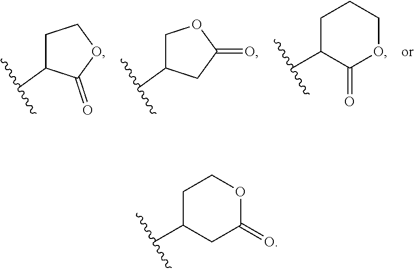

9. The compound of any one of claims 1-8, or a pharmaceutically acceptable salt or solvate thereof, wherein:
ring C is monocyclic carbocycle, or bicyclic carbocycle.

10. The compound of any one of claims 1-9, or a pharmaceutically acceptable salt or solvate thereof, wherein:
ring C is monocyclic carbocycle selected from cyclopropyl, cyclobutyl, cyclopentyl, cyclohexyl, and phenyl.

11. The compound of any one of claims 1-10, or a pharmaceutically acceptable salt or solvate thereof, wherein:
ring C is phenyl.

12. The compound of any one of claims 1-8, or a pharmaceutically acceptable salt or solvate thereof, wherein:
ring C is bicyclic carbocycle selected from indanyl, indenyl, and naphthyl.

13. The compound of any one of claims 1-8, or a pharmaceutically acceptable salt or solvate thereof, wherein:
ring C is monocyclic heterocycle, or bicyclic heterocycle.

14. The compound of any one of claims 1-8, or a pharmaceutically acceptable salt or solvate thereof, wherein:
ring C is monocyclic heterocycle, or bicyclic heterocycle selected from pyridinyl, pyrazolyl, pyrrolyl, imidazolyl, oxazolyl, isoxazolyl, thiazolyl, isothiazolyl, triazolyl, pyrimidinyl, pyrazinyl, triazinyl, benzimidazolyl, indolyl, quinolinyl, indazolyl, purinyl, quinoxalinyl, and acridinyl.

15. The compound of any one of claims 1-8, or a pharmaceutically acceptable salt or solvate thereof, wherein:

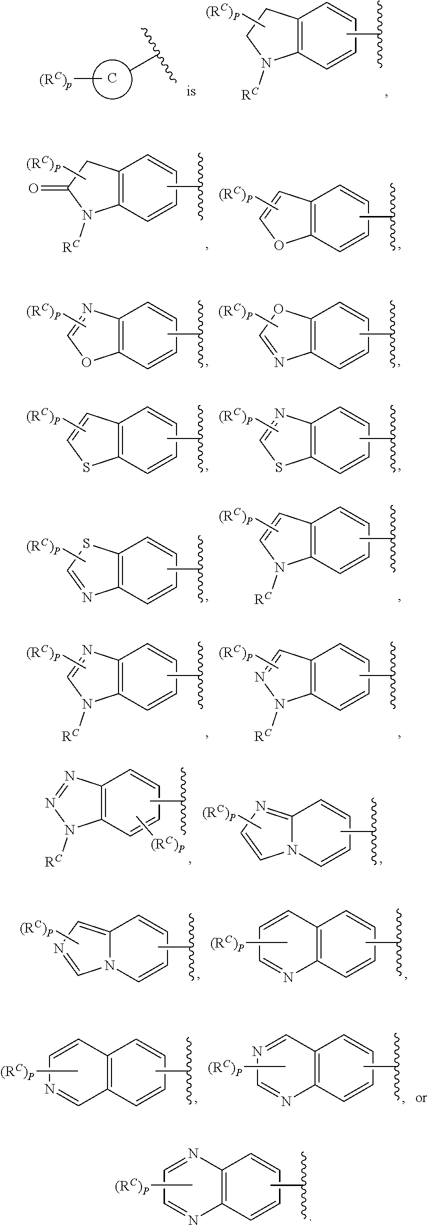

16. The compound of any one of claims 1-8, or a pharmaceutically acceptable salt or solvate thereof, wherein:
ring C is monocyclic heteroaryl selected from furanyl, thienyl, pyridinyl, pyrazolyl, pyrrolyl, imidazolyl, oxazolyl, isoxazolyl, thiazolyl, isothiazolyl, triazolyl, pyrimidinyl, pyrazinyl, and triazinyl.

17. The compound of any one of claims 1-8, or a pharmaceutically acceptable salt or solvate thereof, wherein:
ring C is a monocyclic 6-membered heteroaryl containing 1-3 N atoms.

18. The compound of claim 17, or a pharmaceutically acceptable salt or solvate thereof, wherein:

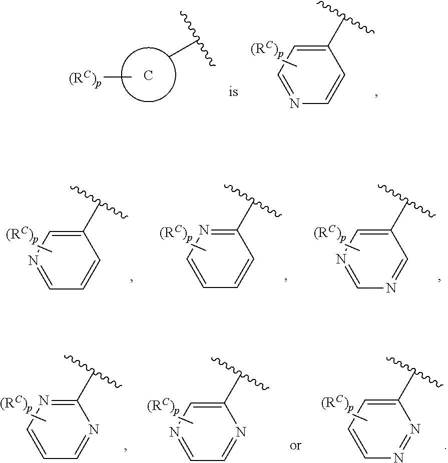

19. The compound of any one of claims 1-8, or a pharmaceutically acceptable salt or solvate thereof, wherein:
ring C is a monocyclic 5-membered C₁-C₄heteroaryl.

20. The compound of claim 19, or a pharmaceutically acceptable salt or solvate thereof, wherein:
ring C is pyrrolyl, oxazolyl, thiazolyl, imidazolyl, pyrazolyl, triazolyl, tetrazolyl, isoxazolyl, isothiazolyl, oxadiazolyl, or thiadiazolyl.

21. The compound of any one of claims 1-8, or a pharmaceutically acceptable salt or solvate thereof, wherein:

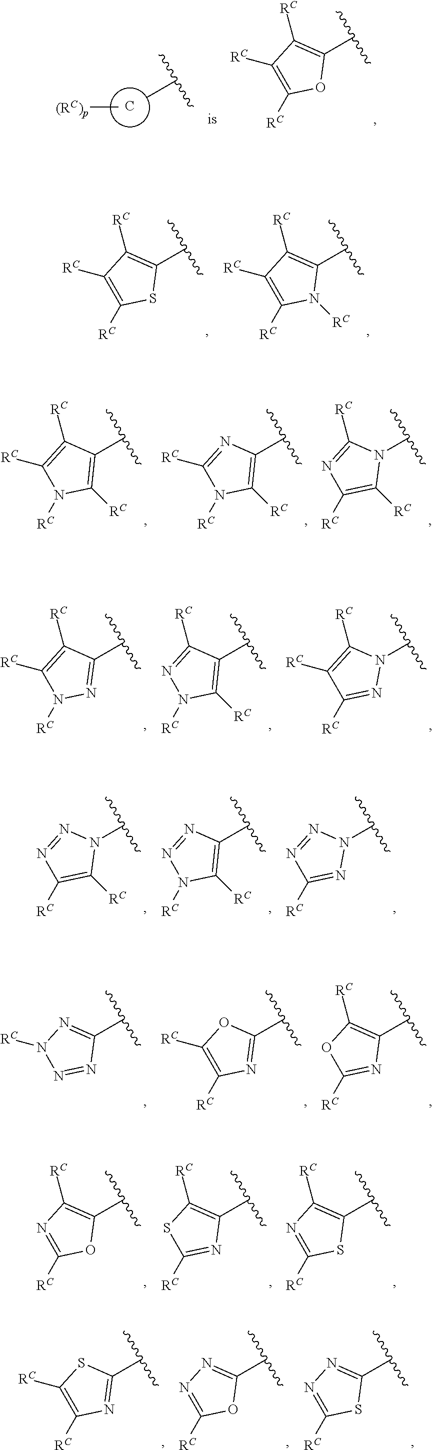

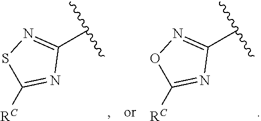

22. The compound of any one of claims 1-8, or a pharmaceutically acceptable salt or solvate thereof, wherein:
ring C is monocyclic heterocycle selected from pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, and 1,2,3,6-tetrahydropyridinyl.

23. The compound of any one of claims 1-8, or a pharmaceutically acceptable salt or solvate thereof, wherein:
ring C is a monocyclic C₂-C₈heterocycloalkyl containing at least 1 N atom in the ring.

24. The compound of claim 23, or a pharmaceutically acceptable salt or solvate thereof, wherein:
ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, or azepanyl.

25. The compound of claim 23, or a pharmaceutically acceptable salt or solvate thereof, wherein: 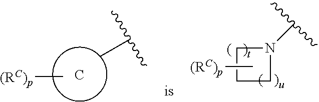
wherein,
t is 1, 2, or 3;
u is 1, 2, or 3.

26. The compound of claim 23, or a pharmaceutically acceptable salt or solvate thereof, wherein:

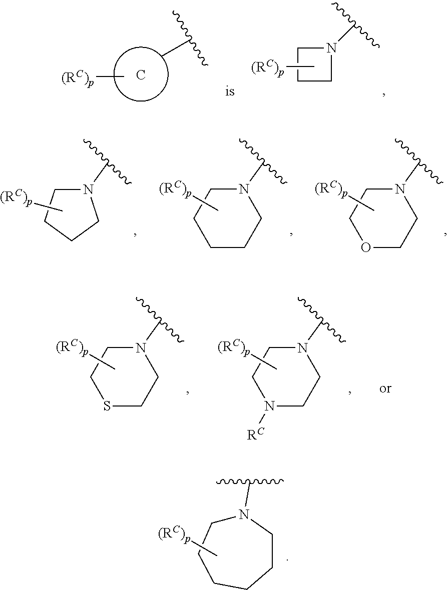

27. The compound of any one of claims of any one of claims 1-26, or a pharmaceutically acceptable salt or solvate thereof, wherein:
R³ is selected from substituted or unsubstituted C₁-C₁₀alkyl, substituted or unsubstituted C₃-C₁₀cycloalkyl, or substituted or unsubstituted aryl, wherein if R³ is substituted then R³ is substituted with one or more R¹² groups.

28. The compound of any one of claims of any one of claims 1-26, or a pharmaceutically acceptable salt or solvate thereof, wherein:
R³ is selected from methyl, ethyl, propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, neohexyl, substituted or unsubstituted cyclopropyl, substituted or unsubstituted cyclobutyl, substituted or unsubstituted cyclopentyl, substituted or unsubstituted cyclohexyl, substituted or unsubstituted phenyl, 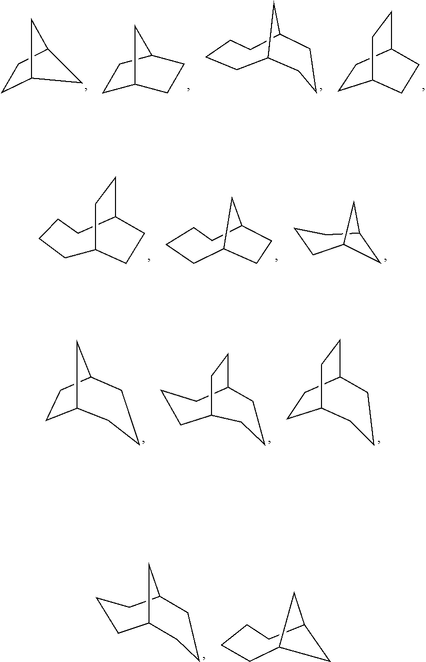
and adamantyl.

29. The compound of claim 1, wherein the compound has the structure of Formula (IX), or a pharmaceutically acceptable salt or solvate thereof: 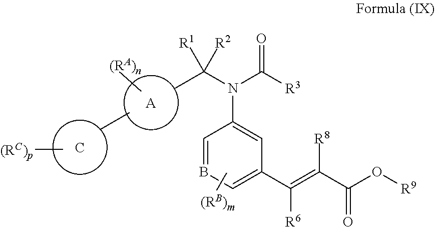
wherein,
R¹ and R² are each independently selected from H, D, F, C₁-C₄alkyl, or C₁-C₄fluoroalkyl;
R³ is selected from substituted or unsubstituted C₁-C₁₀alkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NR¹⁰C(═O)R¹¹, —NR¹⁰C(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
L⁴ is absent, —O—, —S—, —S(═O)—, —S(═O)₂, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
L⁵ is absent, unsubstituted or substituted C1-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
R¹³ is H, halogen, —N(R¹⁰)₂, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁶ and R⁸ are each independently selected from H, D, C₁-C₄alkyl, and C₃-C₆cycloalkyl;
R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, and substituted or unsubstituted heterocycle;
ring A is a monocyclic C₃-C₆cycloalkyl;
each R^{A} is independently selected from H, D, halogen, —CN, —OH, —OR¹⁰, —SR¹¹, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
B is CR^{B} or N;
each R^{B} is independently selected from H, D, halogen, —CN, —OH, —OR¹⁰, —SR¹⁰, —S(═O)R¹¹, —S(═O)₂R¹¹, —N(R¹⁰)₂, —NHS(═O)₂R¹¹, —S(═O)₂N(R¹)₂, —C(═O)R¹¹, —OC(═O)R¹¹, —CO₂R¹⁰, —OCO₂R¹¹, —C(═O)N(R)₂, —OC(═O)N(R¹⁰)₂, —NR¹⁰C(═O)N(R¹⁰)₂, —NR¹⁰C(═O)R¹¹, —NR¹¹C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, and substituted or unsubstituted C₁-C₆heteroalkyl;
ring C is monocyclic carbocycle or monocyclic heterocycle;
each R^{C} is independently selected from H, D, halogen, —CN, —OH, —OR¹⁰, —SR¹¹, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹)₂, —OC(═O)N(R¹⁰)₂, —NR¹⁰C(═O)N(R¹⁰)₂, —NR¹⁰C(═O)R¹¹, —NR¹¹C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl, and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

30. The compound of claim 29, or a pharmaceutically acceptable salt or solvate thereof, wherein:
ring A is

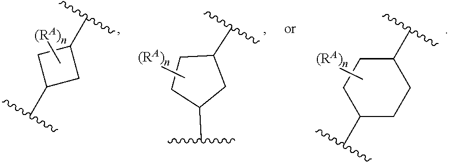

31. The compound of claim 29 or claim 30, or a pharmaceutically acceptable salt or solvate thereof, wherein:
ring C is phenyl.

32. The compound of claim 29 or claim 30, or a pharmaceutically acceptable salt or solvate thereof, wherein:
ring C is monocyclic heterocycle, or bicyclic heterocycle selected from pyridinyl, pyrazolyl, pyrrolyl, imidazolyl, oxazolyl, isoxazolyl, thiazolyl, isothiazolyl, triazolyl, pyrimidinyl, pyrazinyl, triazinyl, benzimidazolyl, indolyl, quinolinyl, indazolyl, purinyl, quinoxalinyl, and acridinyl.

33. The compound of claim 29 or claim 30, or a pharmaceutically acceptable salt or solvate thereof wherein:

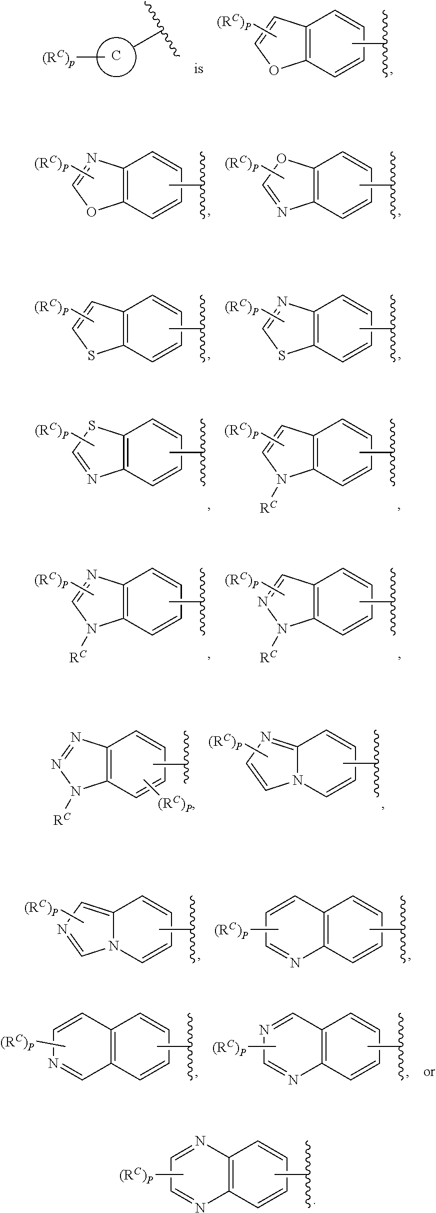

34. The compound of claim 29 or claim 30, or a pharmaceutically acceptable salt or solvate thereof, wherein:
ring C is monocyclic heteroaryl selected from furanyl, thienyl, pyridinyl, pyrazolyl, pyrrolyl, imidazolyl, oxazolyl, isoxazolyl, thiazolyl, isothiazolyl, triazolyl, pyrimidinyl, pyrazinyl, and triazinyl.

35. The compound of claim 29 or claim 30, or a pharmaceutically acceptable salt or solvate thereof, wherein:
ring C is a monocyclic 6-membered heteroaryl containing 1-3 N atoms.

36. The compound of claim 35, or a pharmaceutically acceptable salt or solvate thereof, wherein

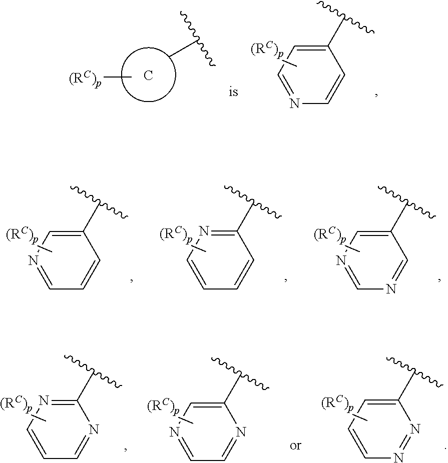

37. The compound of claim 29 or claim 30, or a pharmaceutically acceptable salt or solvate thereof, wherein:
ring C is a monocyclic 5-membered C₁-C₄heteroaryl.

38. The compound of claim 37, or a pharmaceutically acceptable salt or solvate thereof, wherein:
ring C is pyrrolyl, oxazolyl, thiazolyl, imidazolyl, pyrazolyl, triazolyl, tetrazolyl, isoxazolyl, isothiazolyl, oxadiazolyl, or thiadiazolyl.

39. The compound of any one of claims of any one of claims 29-38, or a pharmaceutically acceptable salt or solvate thereof, wherein:
R³ is selected from substituted or unsubstituted C₁-C₁₀alkyl, substituted or unsubstituted C₃-C₁₀cycloalkyl, or substituted or unsubstituted aryl, wherein if R³ is substituted then R³ is substituted with one or more R¹² groups.

40. The compound of any one of claims of any one of claims 29-39, or a pharmaceutically acceptable salt or solvate thereof, wherein:
R³ is selected from methyl, ethyl, propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, neohexyl, substituted or unsubstituted cyclopropyl, substituted or unsubstituted cyclobutyl, substituted or unsubstituted cyclopentyl, substituted or unsubstituted cyclohexyl, and substituted or unsubstituted phenyl.

41. The compound of claim 1, wherein the compound is:
trans-(E)-3-(3-(N-((4-(4-Methoxy-3,3-methylphenyl)cyclohexyl)methyl)cyclohexanecarboxamido)phenyl)acrylic acid;
(E)-Methyl 3-(3-(N-((4-(4-chlorophenyl)cyclohexyl)methyl)cyclohexanecarboxamido)phenyl)acrylate;
trans-(E)-Methyl 3-(3-(N-((4-(4-methoxy-3-methylphenyl)cyclohexyl)methyl)cyclohexanecarboxamido)phenyl)acrylate;
trans-(E)-Methyl 3-(3-(N-((4-(4-methoxy-3-methylphenyl)cyclohexyl)methyl)-3,3-dimethylbutanamido)phenyl)acrylate;
trans-(E)-Isopropyl 3-(3-(N-((4-(4-methoxy-3-methylphenyl)cyclohexyl)methyl)cyclohexanecarboxamido)phenyl)acrylate;
trans-(E)-Isopropyl 3-(3-(N-((4-(4-methoxy-3-methylphenyl)cyclohexyl)methyl)-3,3-dimethylbutanamido)phenyl)acrylate;
cis-(E)-Methyl 3-(3-(N-((4-(4-methoxy-3-methylphenyl)cyclohexyl)methyl)cyclohexanecarboxamido)phenyl)acrylate;
trans-(E)-Isopropyl 3-(3-(trans-4-hydroxy-N-((4-(4-methoxy-3-methylphenyl)cyclohexyl)methyl)cyclohexanecarboxamido)phenyl)acrylate;
trans-(E)-Methyl 3-(3-(trans-4-hydroxy-N-((4-(4-methoxy-3-methylphenyl)cyclohexyl)methyl)cyclohexanecarboxamido)phenyl)acrylate;
or a pharmaceutically acceptable salt or solvate thereof.

42. A compound that has the structure of Formula (I), or a pharmaceutically acceptable salt or solvate thereof: 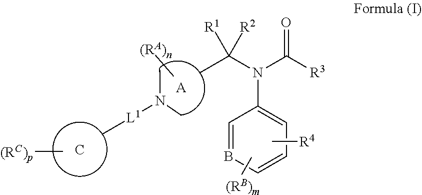
wherein,
R¹ and R² are each independently selected from H, D, F, C₁-C₄alkyl, or C₁-C₄fluoroalkyl;
or R¹ and R² are taken together with the carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C₁-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R¹⁰, —OCO₂R¹¹, —N(R¹⁰)₂, —C(═O)N(R¹⁰)₂, —OC(═O)N(R¹⁰)₂, —NR¹⁰C(═O)R¹¹, —NR¹⁰C(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
L⁴ is absent, —O—, —S—, —S(═O)—, —S(═O)₂—, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
L⁵ is absent, unsubstituted or substituted C1-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
R¹³ is H, halogen, —N(R¹⁰)₂, unsubstituted or substituted C1-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
L¹ is —X¹-L²-, or -L²-X¹—;
X¹ is absent, —S(═O)—, —S(═O)₂—, —C(═O)—, —OC(═O)—, —NR¹⁰C(═O)—, or —NR¹OS(═O)₂—;
L² is absent or substituted or unsubstituted C₁-C₄alkylene;
R⁴ is -L³-Y;
L³ is —C(R⁵)(R⁶)—, —C(R⁵)(R⁶)—C(R⁷)(R⁸)—, —O—C(R⁷)(R⁸)—, or —C(R⁵)(R⁶)—O—;
R⁵ and R⁷ are each independently selected from H, D, C₁-C₄alkyl and C₃-C₆cycloalkyl;
or R⁵ and R⁷ are taken together with the intervening atoms to form a double bond;
or R⁵ and R⁷ are taken together with the intervening atoms to form an epoxide or an substituted or unsubstituted C₃-C₆cycloalkyl;
R⁶ and R⁸ are each independently selected from H, D, C₁-C₄alkyl or C₃-C₆cycloalkyl;
Y is —CH₂OR⁹, —C(═O)OR⁹, 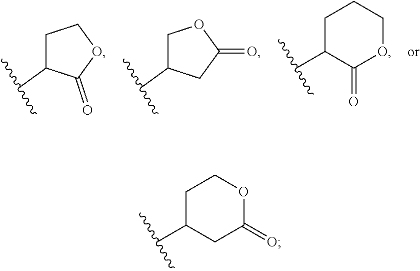 [SMILES: *C1CCOC1=O ; *C1COC(=O)C1 ; *C1CCCOC1=O ; "or" ; *C1CCOC(=O)C1]
R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, or substituted or substituted or unsubstituted heterocycle;
ring A is a monocyclic C₂-C₈heterocycloalkyl containing 1 N atom in the ring, or bicyclic C₅-C₈heterocycloalkyl;
each R^{A} is independently selected from H, D, halogen, —CN, —OH, —OR¹⁰, —SR¹¹, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
B is CR^{B}, or N;
each R^{B} is independently selected from H, D, halogen, —CN, —OH, —OR¹⁰, —SR¹¹, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹¹, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹¹, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

43. The compound of claim 42, or a pharmaceutically acceptable salt or solvate thereof, wherein:
ring A is a monocyclic C₂-C₈heterocycloalkyl containing 1 N atom in the ring that is selected from azetidinyl, pyrrolidinyl, piperidinyl, or azepanyl.

44. The compound of claim 42 or claim 43, or a pharmaceutically acceptable salt or solvate thereof, wherein: 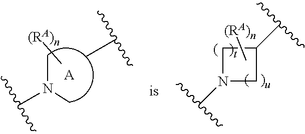
wherein,
t is 1, 2, or 3;
u is 1, 2, or 3.

45. The compound of claim 44, or a pharmaceutically acceptable salt or solvate thereof, wherein:

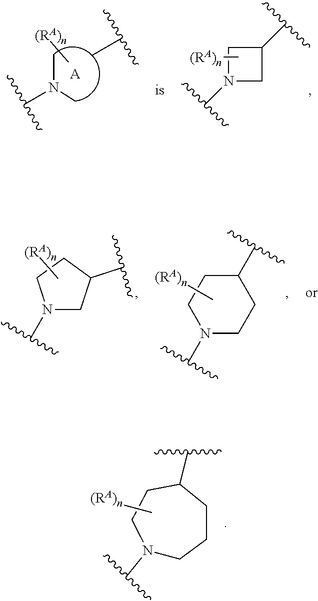

46. The compound of claim 42, or a pharmaceutically acceptable salt or solvate thereof, wherein:
ring A is a monocyclic C₂-C₈heterocycloalkyl containing 1 N atom in the ring that is selected from a β-lactam, γ-lactam, δ-lactam or ε-lactam.

47. The compound of claim 42, or a pharmaceutically acceptable salt or solvate thereof, wherein:
ring A is a bicyclic C₅-C₈heterocycloalkyl that is a fused bicyclic C₅-C₈heterocycloalkyl, bridged bicyclic C₅-C₈heterocycloalkyl, or spiro bicyclic C₅-C₈heterocycloalkyl.

48. The compound of any one of claims 42-47, or a pharmaceutically acceptable salt or solvate thereof, wherein:
X¹ is absent, —S(═O)—, —S(═O)₂—, —C(═O)—, —OC(═O)—, —NR¹⁰C(═O)—, or —NR¹⁰S(═O)₂—;
L² is absent or —CH₂—.

49. The compound of any one of claims 42-48, or a pharmaceutically acceptable salt or solvate thereof, wherein:
L¹ is absent, —CH₂—, —S(═O)₂—, or —C(═O)—.

50. The compound of any one of claims 42-49, or a pharmaceutically acceptable salt or solvate thereof, wherein:
R⁴ is

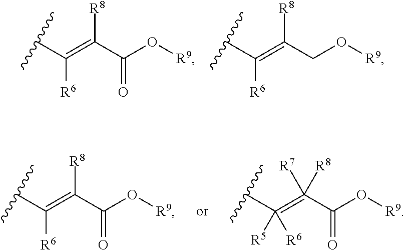

51. The compound of claim 50, or a pharmaceutically acceptable salt or solvate thereof, wherein:
R⁴ is

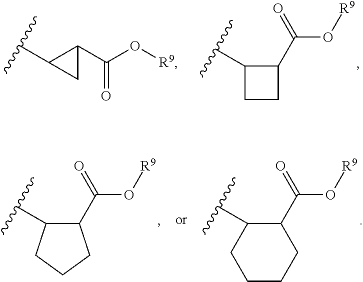

52. The compound of any one of claims 42-49, or a pharmaceutically acceptable salt or solvate thereof, wherein:
R⁴ is -L³-Y;
L³ is —CH₂—
Y is

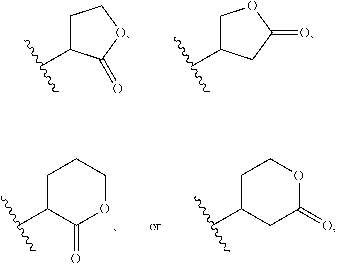 [SMILES: *C1CCOC1=O ; *C1COC(=O)C1 ; *C1CCCOC1=O ; *C1CCOC(=O)C1]

53. The compound of any one of claims 42-52, or a pharmaceutically acceptable salt or solvate thereof, wherein:
ring C is monocyclic carbocycle, or bicyclic carbocycle.

54. The compound of any one of claims 42-53, or a pharmaceutically acceptable salt or solvate thereof, wherein:
ring C is monocyclic carbocycle selected from cyclopropyl, cyclobutyl, cyclopentyl, cyclohexyl, and phenyl.

55. The compound of any one of claims 42-54, or a pharmaceutically acceptable salt or solvate thereof, wherein:
ring C is phenyl.

56. The compound of any one of claims 42-52, or a pharmaceutically acceptable salt or solvate thereof, wherein:
ring C is bicyclic carbocycle selected from indanyl, indenyl, and naphthyl.

57. The compound of any one of claims 42-52, or a pharmaceutically acceptable salt or solvate thereof, wherein:
ring C is monocyclic heterocycle, or bicyclic heterocycle.

58. The compound of any one of claims 42-52, or a pharmaceutically acceptable salt or solvate thereof, wherein:
ring C is monocyclic heterocycle, or bicyclic heterocycle selected from pyridinyl, pyrazolyl, pyrrolyl, imidazolyl, oxazolyl, isoxazolyl, thiazolyl, isothiazolyl, triazolyl, pyrimidinyl, pyrazinyl, triazinyl, benzimidazolyl, indolyl, quinolinyl, indazolyl, purinyl, quinoxalinyl, and acridinyl.

59. The compound of any one of claims 42-52, or a pharmaceutically acceptable salt or solvate thereof, wherein:

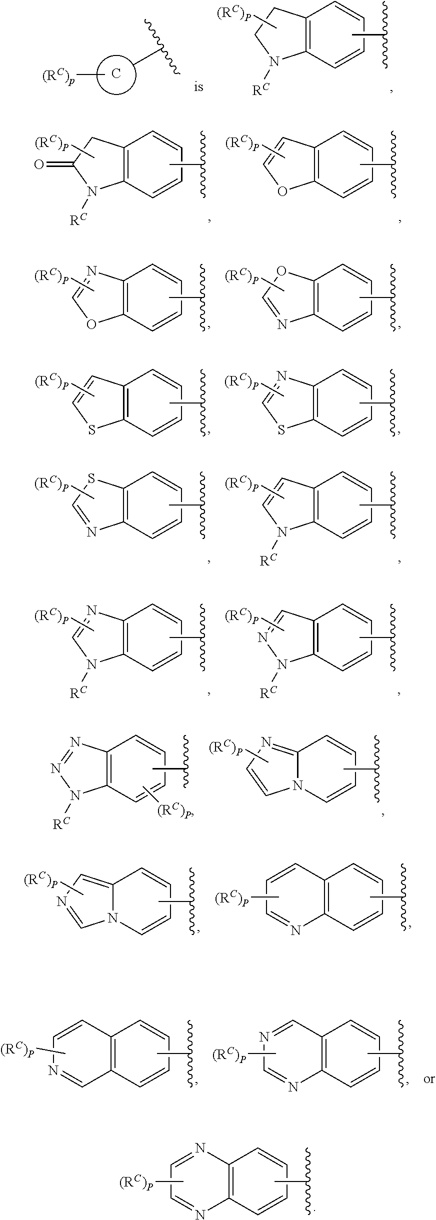

60. The compound of any one of claims 42-52, or a pharmaceutically acceptable salt or solvate thereof, wherein:
ring C is monocyclic heteroaryl selected from furanyl, thienyl, pyridinyl, pyrazolyl, pyrrolyl, imidazolyl, oxazolyl, isoxazolyl, thiazolyl, isothiazolyl, triazolyl, pyrimidinyl, pyrazinyl, and triazinyl.

61. The compound of any one of claims 42-52, or a pharmaceutically acceptable salt or solvate thereof, wherein:
ring C is a monocyclic 6-membered heteroaryl containing 1-3 N atoms.

62. The compound of any one of claims 42-52, or a pharmaceutically acceptable salt or solvate thereof, wherein:

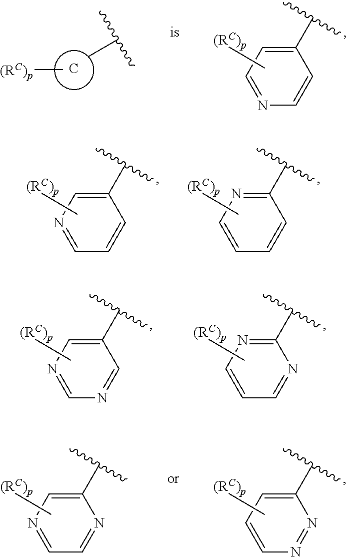

63. The compound of any one of claims 42-52, or a pharmaceutically acceptable salt or solvate thereof, wherein:
ring C is a monocyclic 5-membered C₁-C₄heteroaryl containing 1-4 N atoms, 0 or 1 O or S atom.

64. The compound of claim 63, or a pharmaceutically acceptable salt or solvate thereof, wherein:
ring C is pyrrolyl, oxazolyl, thiazolyl, imidazolyl, pyrazolyl, triazolyl, tetrazolyl, isoxazolyl, isothiazolyl, oxadiazolyl, or thiadiazolyl.

65. The compound of any one of claims 42-52, or a pharmaceutically acceptable salt or solvate thereof, wherein:

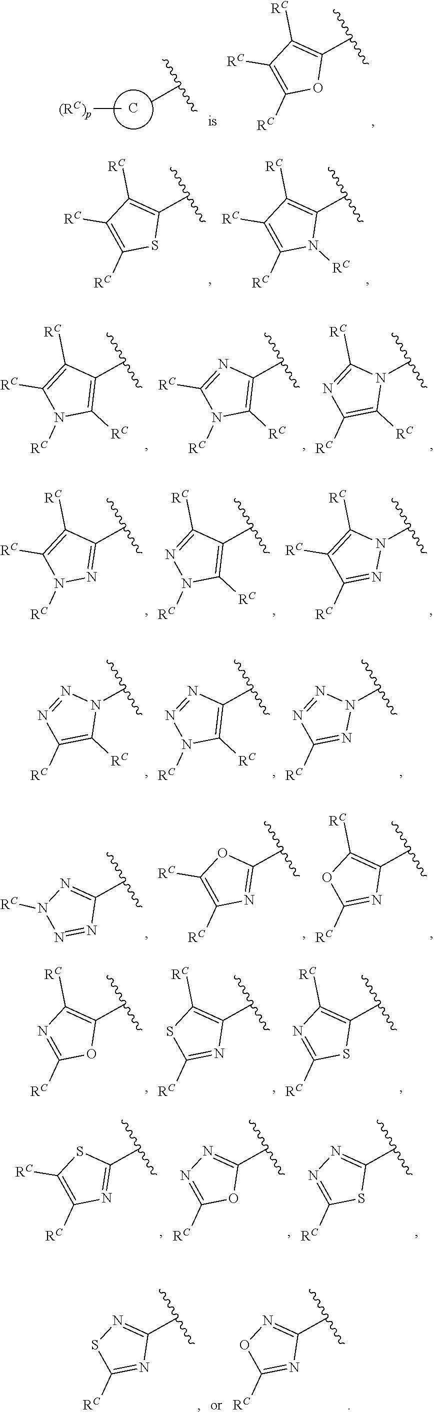

66. The compound of any one of claims 42-52, or a pharmaceutically acceptable salt or solvate thereof, wherein:
ring C is monocyclic heterocycle selected from pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, and 1,2,3,6-tetrahydropyridinyl.

67. The compound of any one of claims 42-52, or a pharmaceutically acceptable salt or solvate thereof, wherein:
ring C is a monocyclic C₂-C₈heterocycloalkyl containing at least 1 N atom in the ring.

68. The compound of claim 67, or a pharmaceutically acceptable salt or solvate thereof, wherein:
ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, or azepanyl.

69. The compound of any one of claims 42-68, or a pharmaceutically acceptable salt or solvate thereof, wherein:
R³ is selected from substituted or unsubstituted C₁-C₁₀alkyl, substituted or unsubstituted C₃-C₁₀cycloalkyl, or substituted or unsubstituted aryl, wherein if R³ is substituted then R³ is substituted with one or more R¹² groups.

70. The compound of any one of claims 42-69, or a pharmaceutically acceptable salt or solvate thereof, wherein:
R³ is selected from methyl, ethyl, propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, neohexyl, substituted or unsubstituted cyclopropyl, substituted or unsubstituted cyclobutyl, substituted or unsubstituted cyclopentyl, substituted or unsubstituted cyclohexyl, substituted or unsubstituted phenyl, 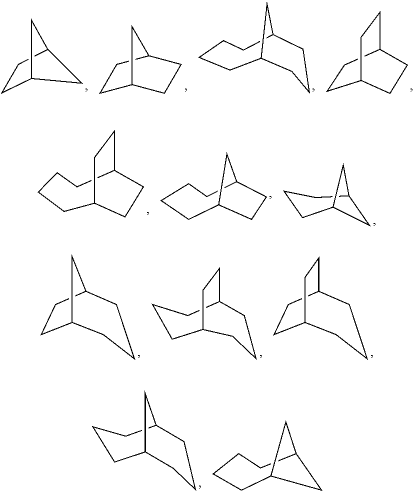
and adamantyl.

71. The compound of claim 42, wherein the compound is:
(E)-Methyl 3-(3-(N-((1-(4-(dimethylamino)phenyl)piperidin-4-yl)methyl) cyclohexanecarboxamido)phenyl) acrylate;
(R,E)-Methyl 3-(3-(N-((1-(4-(dimethylamino)phenyl)pyrrolidin-3-yl)methyl) cyclohexanecarboxamido)phenyl) acrylate;
(R,E)-Methyl 3-(3-(N-((1-(4-(dimethylamino)phenyl)pyrrolidin-3-yl)methyl) cyclohexanecarboxamido)phenyl) acrylate;
(E)-Methyl 3-(3-(N-((1-(4-(dimethyl amino)phenyl)azetidin-3-yl)methyl) cyclohexanecarboxamido)phenyl) acrylate;
(E)-Methyl 3-(3-(N-((1-(p-tolyl)piperidin-4-yl)methyl)cyclohexanecarboxamido)phenyl) acrylate;
(E)-3-(3-(N-((1-(4-(Dimethylamino)phenyl)piperidin-4-yl)methyl) cyclohexanecarboxamido)phenyl)acrylic acid;
or a pharmaceutically acceptable salt or solvate thereof.

72. A compound that has the structure of Formula (II), or a pharmaceutically acceptable salt or solvate thereof: 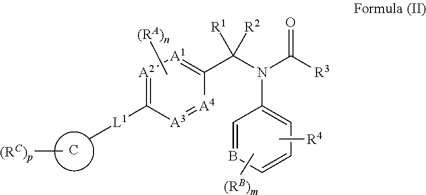
wherein,
A¹ is CR^{A};
or A¹ is N if at least one of A², A³, or A⁴ is N;
A² is CR^{A} or N;
A³ is CR^{A} or N;
A⁴ is CR^{A};
or A⁴ is N if at least one of A A², or A³ is N
provided that at least one of A¹, A², A³, or A⁴ is N;
each R^{A} is independently selected from H, D, halogen, —CN, —OH, —OR¹⁰, —SR¹⁰, —S(═O)R¹¹, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
R¹ and R² are each independently selected from H, D, F, C₁-C₄alkyl, or C₁-C₄fluoroalkyl;
or R¹ and R² are taken together with the carbon atom to which they are attached to form a substituted or unsubstituted C₃-C₁₀cycloalkyl, or substituted or unsubstituted C₂-C₁₀heterocycloalkyl;
or R¹ and R² are taken together with the carbon atom to which they are attached to form a carbonyl (C═O);
R³ is selected from substituted or unsubstituted C1-C₁₀alkyl, substituted or unsubstituted C₂-C₁₀alkenyl, substituted or unsubstituted C₂-C₁₀alkynyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl, wherein if R³ is substituted then R³ is substituted with one or more R¹² groups;
each R¹² is independently selected from D, halogen, —CN, —NO₂, —OR¹⁰, —SR¹⁰, —S(═O)R¹¹, —S(═O)₂R¹¹, —S(═O)₂N(R¹⁰)₂, —NR¹⁰S(═O)₂R¹¹, —C(═O)R¹¹, —OC(═O)R¹¹, —CO₂R⁰, —OCO₂R¹¹, —N(R¹⁰)₂, —C(═O)N(R¹⁰)₂, —OC(═O)N(R¹⁰)₂, —NR¹⁰C(═O)R¹¹, —NR¹¹C(═O)OR¹¹, unsubstituted or substituted C₁-C₁₀alkyl, unsubstituted or substituted C₁-C₁₀fluoroalkyl, unsubstituted or substituted C₂-C₁₀alkenyl, unsubstituted or substituted C₂-C₁₀alkynyl, unsubstituted or substituted C₁-C₁₀heteroalkyl, unsubstituted or substituted C₃-C₁₀cycloalkyl, unsubstituted or substituted aryl, unsubstituted or substituted heteroaryl, and -L⁴-L⁵-R¹³;
L⁴ is absent, —O—, —S—, —S(═O)—, —S(═O)₂, —NR¹⁰—, —C(═O)—, —C(═O)NH—, —NHC(═O)—, —C(═O)O—, —OC(═O)—, —OC(═O)NH—, —NHC(═O)NH—, —NHC(═O)O—, —(CH₂)ᵣ—, or —(OCH₂CH₂)ᵣ—, r is 1, 2, 3, or 4;
L⁵ is absent, unsubstituted or substituted C1-C₁₀alkylene, unsubstituted or substituted C₁-C₁₀heteroalkylene, unsubstituted or substituted C₂-C₁₀alkenylene, unsubstituted or substituted C₂-C₁₀alkynylene, unsubstituted or substituted C₃-C₁₀cycloalkylene, unsubstituted or substituted C₂-C₁₀heterocycloalkylene, unsubstituted or substituted arylene, or unsubstituted or substituted heteroarylene;
R¹³ is H, halogen, —N(R¹⁰)₂, unsubstituted or substituted C₁-C₁₀alkyl, unsubstituted or substituted C₁-C₁₀alkenyl, unsubstituted or substituted C₁-C₁₀alkynyl, unsubstituted or substituted C₁-C₁₀cycloalkyl, unsubstituted or substituted C₁-C₁₀heterocycloalkyl, unsubstituted or substituted aryl, or unsubstituted or substituted heteroaryl;
R⁴ is -L³-Y;
L³ is —C(R⁵)(R⁶)—, —C(R⁵)(R⁶)—C(R⁷)(R⁸)—, —O—C(R^{T})(R⁸)—, or —C(R⁵)(R⁶)—O—;
R⁵ and R⁷ are each independently selected from H, D, C₁-C₄alkyl and C₃-C₆cycloalkyl;
or R⁵ and R⁷ are taken together with the intervening atoms to form a double bond;
or R⁵ and R⁷ are taken together with the intervening atoms to form an epoxide or an substituted or unsubstituted C₃-C₆cycloalkyl;
R⁶ and R⁸ are each independently selected from H, D, C₁-C₄alkyl or C₃-C₆cycloalkyl;
Y is —CH₂OR⁹, 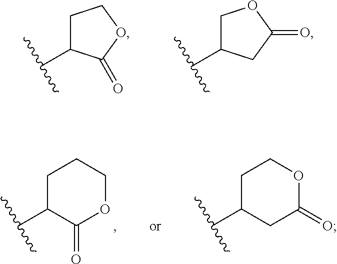 [SMILES: *C1CCOC1=O ; *C1COC(=O)C1 ; *C1CCCOC1=O ; *C1CCOC(=O)C1]
R⁹ is selected from H, substituted or unsubstituted C₁-C₆alkyl, substituted or unsubstituted C₁-C₆heteroalkyl, substituted or unsubstituted C₂-C₆alkenyl, substituted or unsubstituted C₃-C₆cycloalkyl, substituted or unsubstituted phenyl, or substituted or substituted or unsubstituted heterocycle;
L¹ is —X¹-L²-, or -L²-X¹;
X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —OC(═O)O—, —C(═O)NR¹⁰—, —NR¹⁰C(═O)—, —OC(═O)NR¹⁰—, —NR¹⁰C(═O)O—, —NR¹⁰C(═O)NR¹—, —NR¹S(═O)₂—, or —NR¹⁰—;
L² is absent or substituted or unsubstituted C₁-C₄alkylene;
B is CR^{B}, or N;
each R^{B} is independently selected from H, D, halogen, —CN, —OH, —OR¹⁰, —SR¹¹, —S(═O)R¹¹, —S(═O)₂R¹¹, —N(R¹⁰)₂, —NHS(═O)₂R¹¹, —S(═O)₂N(R¹⁰)₂, —C(═O)R¹¹, —OC(═O)R¹¹, —CO₂R¹¹, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl;
ring C is monocyclic carbocycle, bicyclic carbocycle, monocyclic N-containing heterocycle, or bicyclic heterocycle;
each R^{C} is independently selected from H, D, halogen, —CN, —OH, —OR¹⁰, —SR¹⁰, —S(═O)R¹¹, —NO₂, —N(R¹⁰)₂, —S(═O)₂R¹¹, —NHS(═O)₂R¹¹, —S(═O)₂N(R¹⁰)₂, —C(═O)R¹¹, —OC(═O)R¹¹, —CO₂R¹⁰, —OCO₂R¹¹, —C(═O)N(R¹⁰)₂, —OC(═O)N(R¹⁰)₂, —NR¹⁰C(═O)N(R¹⁰)₂, —NR¹⁰C(═O)R¹¹, —NR¹⁰C(═O)OR¹¹, substituted or unsubstituted C₁-C₆alkyl, substituted or unsubstituted C₂-C₆alkenyl, substituted or unsubstituted C₂-C₆alkynyl, substituted or unsubstituted C₁-C₆fluoroalkyl, substituted or unsubstituted C₁-C₆heteroalkyl, substituted or unsubstituted phenyl and substituted or unsubstituted monocyclic heteroaryl;
each R¹⁰ is independently selected from H, substituted or unsubstituted C₁-C₆alkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
or two R¹⁰ on the same N atom are taken together with the N atom to which they are attached to form a N-containing heterocycle;
each R¹¹ is independently selected from substituted or unsubstituted C₁-C₆alkyl, substituted or unsubstituted C₁-C₆heteroalkyl, substituted or unsubstituted C₁-C₆fluoroalkyl, substituted or unsubstituted C₃-C₆cycloalkyl, substituted or unsubstituted phenyl, substituted or unsubstituted monocyclic heteroaryl, and substituted or unsubstituted benzyl;
m is 0, 1, or 2;
n is 0, 1, or 2;
p is 0, 1, 2, 3, or 4.

73. The compound of claim 72, or a pharmaceutically acceptable salt or solvate thereof, wherein:
A¹ is CR^{A}; A² is N; A³ is CR^{A} or N; A⁴ is CR^{A} or N.
or A¹ is CR^{A}; A² is N; A³ is CR^{A}; A⁴ is CR^{A};
or A¹ is CR^{A}; A² is N; A³ is N; A⁴ is CR^{A};
or A¹ is CR^{A}; A² is N; A³ is CR^{A}; A⁴ is N;
or A¹ is N; A² is N; A³ is CR^{A}; A⁴ is CR^{A};
or A¹ is N; A² is CR^{A}; A³ is CR^{A}; A⁴ is N.

74. The compound of claim 73, or a pharmaceutically acceptable salt or solvate thereof, wherein:

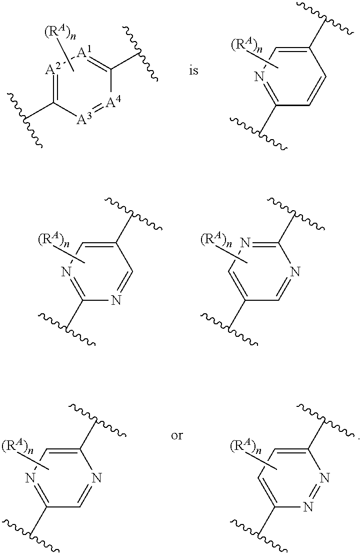

75. The compound of any one of claims 72-74, or a pharmaceutically acceptable salt or solvate thereof, wherein:
L is —X¹-L²-, -L²-X¹—;
X¹ is absent, —O—, —S—, —S(═O)—, —S(═O)₂—, —S(═O)₂NR¹⁰—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, or —NR¹⁰—;
L² is absent or —CH₂—.

76. The compound of any one of claims 72-75, or a pharmaceutically acceptable salt or solvate thereof, wherein:
L¹ is absent, —O—, —S—, —S—CH₂—, —CH₂—S—, —CH₂—, —CH═CH—, —C≡C—, —C(═O)—, —C(═O)O—, —OC(═O)—, —C(═O)NR¹⁰—, —NR¹⁰C(═O)—, —NR¹⁰S(═O)₂—, —NR¹⁰—, —NR¹⁰—CH₂—, or —CH₂—NR¹⁰—.

77. The compound of any one of claims 72-76, or a pharmaceutically acceptable salt or solvate thereof, wherein:
R⁴ is

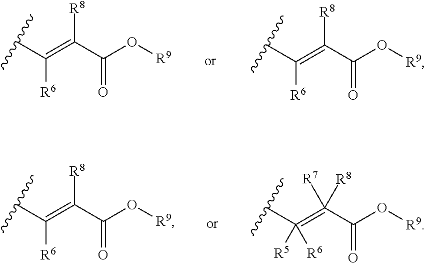

78. The compound of claim 77, or a pharmaceutically acceptable salt or solvate thereof, wherein:
R⁴ is

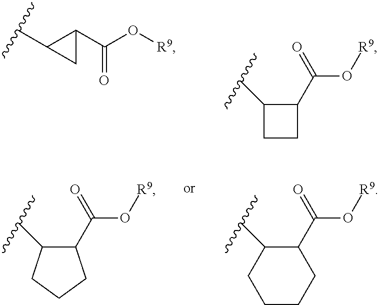

79. The compound of any one of claims 72-76, or a pharmaceutically acceptable salt or solvate thereof, wherein:
R⁴ is -L³-Y;
L³ is —CH₂—;
Y is

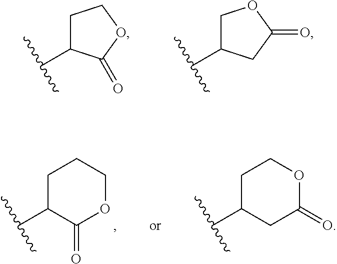 [SMILES: *C1CCOC1=O ; *C1COC(=O)C1 ; *C1CCCOC1=O ; *C1CCOC(=O)C1]

80. The compound of any one of claims 72-79, or a pharmaceutically acceptable salt or solvate thereof, wherein:
ring C is monocyclic carbocycle, or bicyclic carbocycle.

81. The compound of any one of claims 72-79, or a pharmaceutically acceptable salt or solvate thereof, wherein:
ring C is monocyclic carbocycle selected from cyclopropyl, cyclobutyl, cyclopentyl, cyclohexyl, and phenyl.

82. The compound of any one of claims 72-79, or a pharmaceutically acceptable salt or solvate thereof, wherein:
ring C is phenyl.

83. The compound of any one of claims 72-79, or a pharmaceutically acceptable salt or solvate thereof, wherein:
ring C is bicyclic carbocycle selected from indanyl, indenyl, and naphthyl.

84. The compound of any one of claims 72-79, or a pharmaceutically acceptable salt or solvate thereof, wherein:
ring C is monocyclic heterocycle, or bicyclic heterocycle.

85. The compound of any one of claims 72-79, or a pharmaceutically acceptable salt or solvate thereof, wherein:
ring C is monocyclic heterocycle, or bicyclic heterocycle selected from pyridinyl, pyrazolyl, pyrrolyl, imidazolyl, oxazolyl, isoxazolyl, thiazolyl, isothiazolyl, triazolyl, pyrimidinyl, pyrazinyl, triazinyl, benzimidazolyl, indolyl, quinolinyl, indazolyl, purinyl, quinoxalinyl, and acridinyl.

86. The compound of any one of claims 72-79, or a pharmaceutically acceptable salt or solvate thereof, wherein:

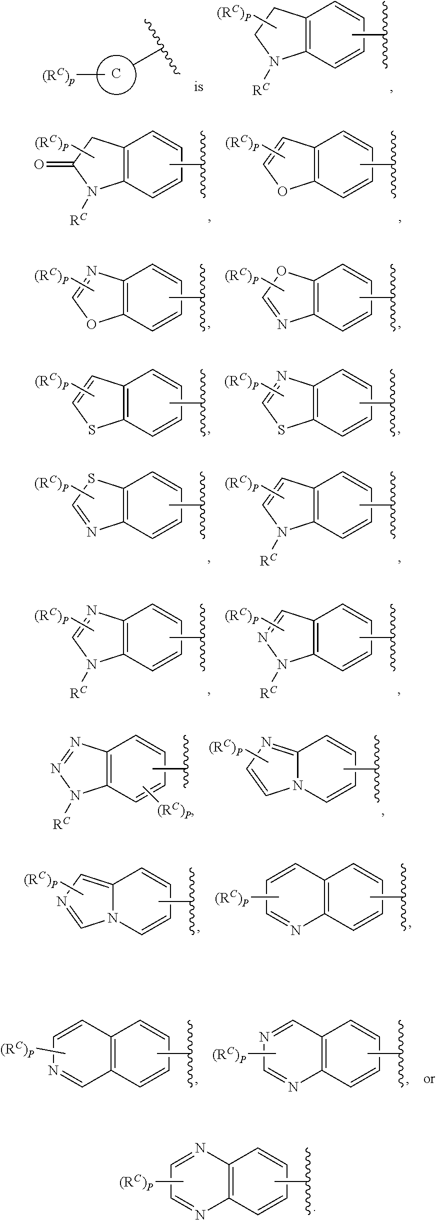

87. The compound of any one of claims 72-79, or a pharmaceutically acceptable salt or solvate thereof, wherein:
ring C is monocyclic heteroaryl selected from furanyl, thienyl, pyridinyl, pyrazolyl, pyrrolyl, imidazolyl, oxazolyl, isoxazolyl, thiazolyl, isothiazolyl, triazolyl, pyrimidinyl, pyrazinyl, and triazinyl.

88. The compound of any one of claims 72-79, or a pharmaceutically acceptable salt or solvate thereof, wherein:
ring C is a monocyclic 6-membered heteroaryl containing 1-3 N atoms.

89. The compound of claim 88, or a pharmaceutically acceptable salt or solvate thereof, wherein:

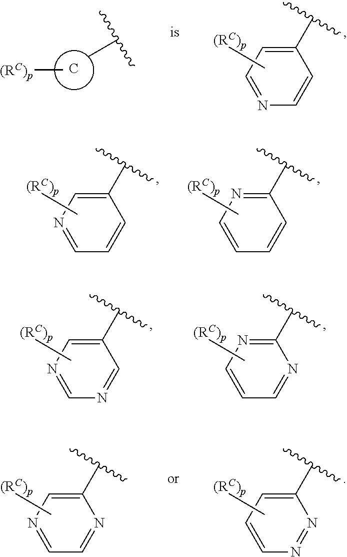

90. The compound of any one of claims 72-79, or a pharmaceutically acceptable salt or solvate thereof, wherein:
ring C is a monocyclic 5-membered C₁-C₄heteroaryl containing 1-4 N atoms, 0 or 1 O or S atom.

91. The compound of claim 90, or a pharmaceutically acceptable salt or solvate thereof, wherein:
ring C is pyrrolyl, oxazolyl, thiazolyl, imidazolyl, pyrazolyl, triazolyl, tetrazolyl, isoxazolyl, isothiazolyl, oxadiazolyl, or thiadiazolyl.

92. The compound of any one of claims 72-79, or a pharmaceutically acceptable salt or solvate thereof, wherein:

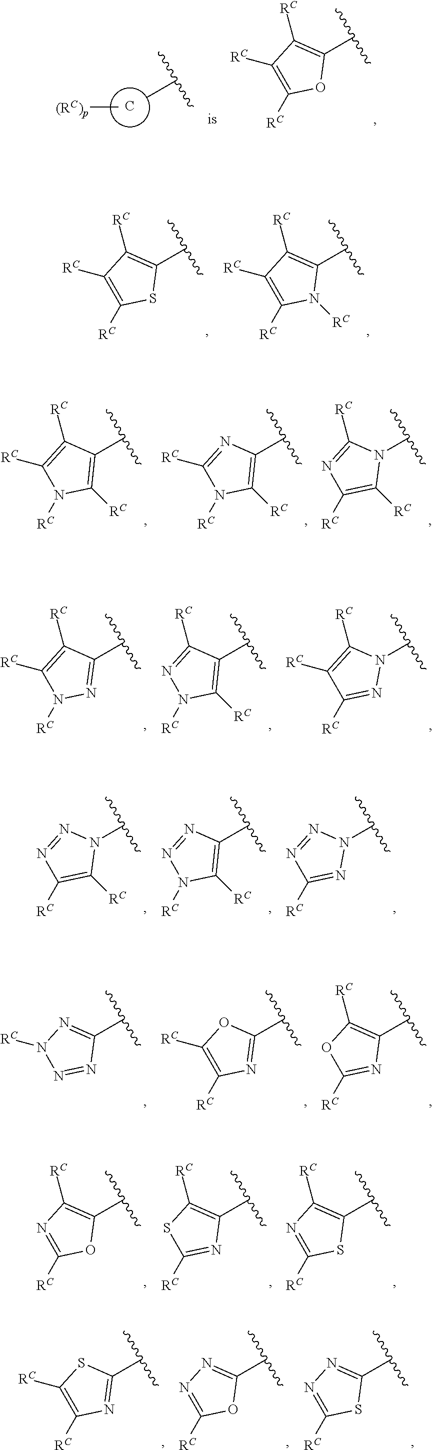

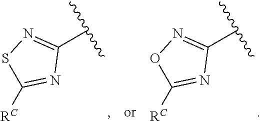

93. The compound of any one of claims 72-79, or a pharmaceutically acceptable salt or solvate thereof, wherein:
ring C is monocyclic heterocycle selected from pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, and 1,2,3,6-tetrahydropyridinyl.

94. The compound of any one of claims 72-79, or a pharmaceutically acceptable salt or solvate thereof, wherein:
ring C is a monocyclic C₂-C₈heterocycloalkyl containing at least 1 N atom in the ring.

95. The compound of claim 94, or a pharmaceutically acceptable salt or solvate thereof, wherein:
ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, or azepanyl.

96. The compound of claim 95, or a pharmaceutically acceptable salt or solvate thereof, wherein:

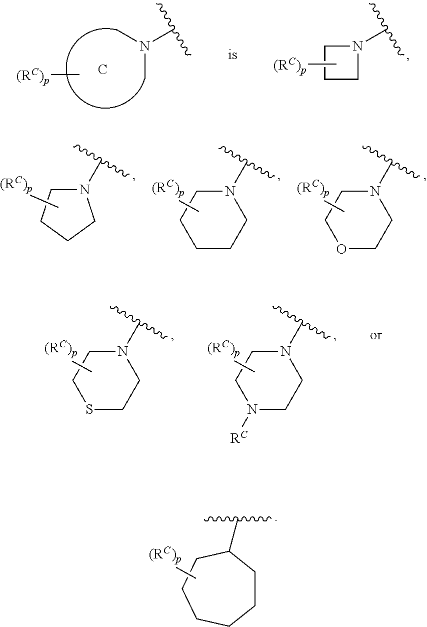

97. The compound of any one of claims 72-79, wherein the compound of Formula (I) has the structure of Formula (III), or a pharmaceutically acceptable salt or solvate thereof: 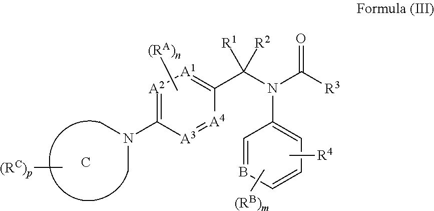
wherein,
ring C is a 5-membered N-containing heteroaryl, or a N-containing C₂-C₈heterocycloalkyl.

98. The compound of claim 97, or a pharmaceutically acceptable salt or solvate thereof, wherein:
ring C is a 5-membered N-containing heteroaryl containing 1-4 N atoms.

99. The compound of claim 98, or a pharmaceutically acceptable salt or solvate thereof, wherein: 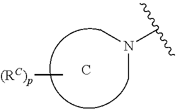
is a monocyclic 5-membered C₁-C₄heteroarylene containing 1-4 N atoms that has the structure

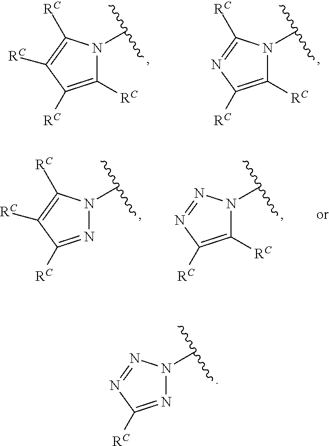

100. The compound of claim 98, or a pharmaceutically acceptable salt or solvate thereof, wherein:
ring C is a monocyclic C₂-C₈heterocycloalkyl containing at least 1 N atom in the ring that is selected from aziridinyl, azetidinyl, pyrrolidinyl, morpholinyl, thiomorpholinyl, piperidinyl, piperazinyl, or azepanyl.

101. The compound of claim 100, or a pharmaceutically acceptable salt or solvate thereof, wherein: 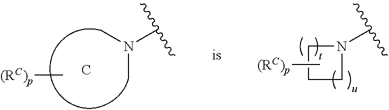
wherein,
t is 1, 2, or 3;
u is 1, 2, or 3.

102. The compound of claim 101, or a pharmaceutically acceptable salt or solvate thereof, wherein:

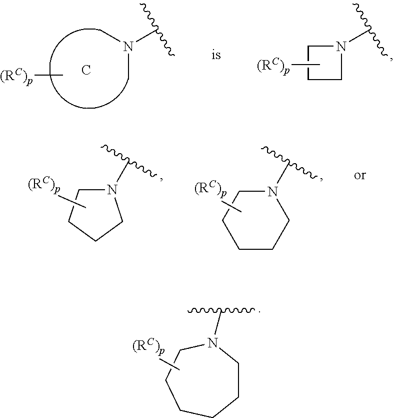

103. The compound of claim 97, or a pharmaceutically acceptable salt or solvate thereof, wherein:
ring C is a monocyclic C₂-C₈heterocycloalkyl containing 1 N atom in the ring that is selected from a β-lactam, γ-lactam, δ-lactam or ε-lactam.

104. The compound of claim 97, or a pharmaceutically acceptable salt or solvate thereof, wherein:
ring C is a bicyclic C₅-C₈heterocycloalkyl that is a fused bicyclic C₅-C₈heterocycloalkyl, bridged bicyclic C₅-C₈heterocycloalkyl, or spiro bicyclic C₅-C₈heterocycloalkyl.

105. The compound of any one of claims 72-104, or a pharmaceutically acceptable salt or solvate thereof, wherein:
R³ is selected from substituted or unsubstituted C₁-C₁₀alkyl, substituted or unsubstituted C₃-C₁₀cycloalkyl, or substituted or unsubstituted aryl, wherein if R³ is substituted then R³ is substituted with one or more R¹² groups.

106. The compound of any one of claims 72-105, or a pharmaceutically acceptable salt or solvate thereof, wherein:
R³ is selected from methyl, ethyl, propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, neohexyl, substituted or unsubstituted cyclopropyl, substituted or unsubstituted cyclobutyl, substituted or unsubstituted cyclopentyl, substituted or unsubstituted cyclohexyl, substituted or unsubstituted phenyl, 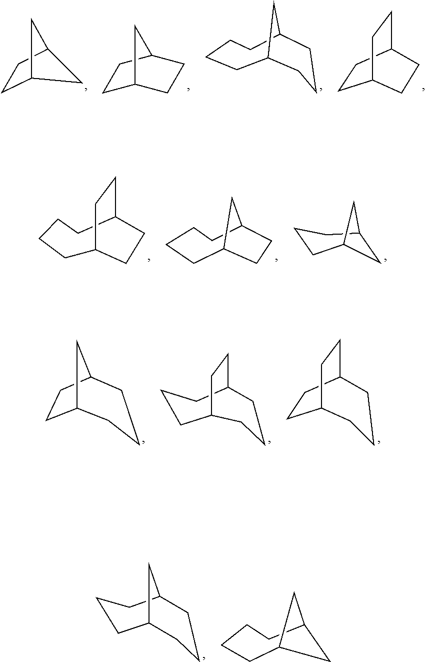
and adamantyl.

107. A pharmaceutical composition comprising a compound of any one of claims 1-106, or a pharmaceutically acceptable salt, or solvate thereof, and at least one pharmaceutically acceptable excipient.

108. The pharmaceutical composition of claim 107, wherein the pharmaceutical composition is formulated for administration to a mammal by intravenous administration, subcutaneous administration, oral administration, inhalation, nasal administration, dermal administration, or ophthalmic administration.

109. The pharmaceutical composition of claim 107, wherein the pharmaceutical composition is in the form of a tablet, a pill, a capsule, a liquid, a suspension, a gel, a dispersion, a solution, an emulsion, an ointment, or a lotion.

110. A method of treating or preventing a liver disease or condition in a subject, comprising administering to the subject a therapeutically effective amount of one or more of the compounds of any one of claims 1-106, or a pharmaceutically acceptable salt or solvate thereof.

111. The method of claim 110, wherein the liver disease or condition is an alcoholic or non-alcoholic liver disease.

112. The method of claim 110, wherein the liver disease or condition is primary biliary cirrhosis, primary sclerosing cholangitis, cholestasis, nonalcoholic steatohepatitis (NASH), or nonalcoholic fatty liver disease (NAFLD)

113. The method of claim 110, wherein the alcoholic liver disease or condition is fatty liver (steatosis), cirrhosis, or alcoholic hepatitis.

114. The method of claim 110, wherein the non-alcoholic liver disease or condition is nonalcoholic steatohepatitis (NASH), or nonalcoholic fatty liver disease (NAFLD).

115. The method of claim 110, wherein the non-alcoholic liver disease or condition is intrahepatic cholestasis or extrahepatic cholestasis.

116. A method of treating or preventing a metabolic disorder in a subject, comprising: administering to a gastrointestinal tract of the subject a therapeutically effective amount of one or more of the compounds of any one of claims 1-106, or a pharmaceutically acceptable salt or solvate thereof, thereby activating farnesoid X receptors (FXR) in the intestines, and treating or preventing a metabolic disorder in the subject.

117. The method of claim 116, wherein the method reduces or prevents diet-induced weight gain.

118. The method of one of claims 116-117, wherein the method increases a metabolic rate in the subject.

119. The method of claim 118, wherein the increasing the metabolic rate comprises enhancing oxidative phosphorylation in the subject.

120. The method of one of claims 116-119, further comprising improving glucose and/or lipid homeostasis in the subject.

121. The method of one of claims 116-120, wherein the metabolic disorder is selected from obesity, diabetes, insulin resistance, dyslipidemia or any combination thereof.

122. The method of one of claims 116-120, wherein the metabolic disorder is non-insulin dependent diabetes mellitus.

123. The method of one of claims 116-120, wherein the method protects against diet-induced weight gain, reduces inflammation, enhances thermogenesis, enhances insulin sensitivity in the liver, reduces hepatic steatosis, promotes activation of brown adipose tissue (BAT), decreases blood glucose, increases weight loss, or any combination thereof.

124. The method of claim 123, wherein the method enhances insulin sensitivity in the liver and promotes brown adipose tissue (BAT) activation.

125. The method of one of claims 116-124, further comprising administering to the subject an insulin sensitizing drug, an insulin secretagogue, an alpha-glucosidase inhibitor, a glucagon-like peptide (GLP) agonist, a dipeptidyl peptidase-4 (DPP-4) inhibitor, nicotinamide ribonucleoside, an analog of nicotinamide ribonucleoside, or combinations thereof.

126. A method of treating or preventing inflammation in an intestinal region of a subject, comprising:
administering to a gastrointestinal tract of the subject a therapeutically effective amount of one or more of the compounds of any one of claims 1-106, or a pharmaceutically acceptable salt or solvate thereof, thereby activating FXR receptors in the intestines, and thereby treating or preventing inflammation in the intestinal region of the subject.

127. The method of claim 126, wherein the inflammation is associated with condition selected from necrotizing enterocolitis, gastritis, ulcerative colitis, Crohn's disease, inflammatory bowel disease, irritable bowel syndrome, gastroenteritis, radiation induced enteritis, pseudomembranous colitis, chemotherapy induced enteritis, gastro-esophageal reflux disease (GERD), peptic ulcer, non-ulcer dyspepsia (NUD), celiac disease, intestinal celiac disease, post-surgical inflammation, gastric carcinogenesis or any combination thereof.

128. The method of one of claim 127, further comprising administering a therapeutically effective amount of an antibiotic therapy to the subject, wherein the method treats or prevents inflammation associated with pseudomembranous colitis in the subject.

129. The method of one of claims 126-128, further comprising administering to the subject a therapeutically effective amount of an oral corticosteroid, other anti-inflammatory or immunomodulatory therapy, nicotinamide ribonucleoside, an analog of nicotinamide ribonucleoside, or combinations thereof.

130. A method of treating or preventing a cell proliferation disease in a subject, comprising administering to a gastrointestinal tract of the subject a therapeutically effective amount of one or more of the compounds of any one of claims 1-106, or a pharmaceutically acceptable salt or solvate thereof.

131. The method of claim 130, wherein the cell proliferation disease is an adenocarcinoma.

132. The method of claim 130, wherein the adenocarcinoma is a colon cancer.

133. The method of claim 131, wherein the treating the adenocarcinoma reduces the size of the adenocarcinoma, the volume of the adenocarcinoma, the number of adenocarcinomas, cachexia due to the adenocarcinoma, delays progression of the adenocarcinoma, increases survival of the subject, or combinations thereof.

134. The method of any of claims 130-133, wherein the method further comprises administering to the subject an additional therapeutic compound selected from the group consisting of a chemotherapeutic, a biologic, a radiotherapeutic, or combinations thereof.
